(19) United States
(12) Patent Application Publication
(10) Pub. No.: US 2010/0048539 A1
(43) Pub. Date: Feb. 25, 2010

(54) COMPOUNDS AND COMPOSITIONS AS PROTEIN KINASE INHIBITORS

(75) Inventors: Donatella Chianelli, San Diego, CA (US); Xiaolin Li, San Diego, CA (US); Xiaodong Liu, San Diego, CA (US); Jon Loren, San Diego, CA (US); Valentina Molteni, San Diego, CA (US); Juliet Nabakka, Santee, CA (US); Laszlo Revesz, Therwil (CH); Lawrence B. Perez, Hopkinton, MA (US); Clinton Brooks, Boston, MA (US); Wojciech Wrona, Waltham, MA (US); Paul William Manley, Arlesheim (CH); Werner Breitenstein, Basel (CH)

Correspondence Address: GENOMICS INSTITUTE OF THE;NOVARTIS RESEARCH FOUNDATION, 10675 JOHN JAY HOPKINS DRIVE, SUITE E225, SAN DIEGO, CA, 92121-1127, US

(73) Assignees: IRM LLC, Hamilton (BM); NOVARTIS AG, Basel (CH)

(21) Appl. No.: 12/513,498

(22) PCT Filed: Nov. 2, 2007
(86) PCT No.: PCT/US07/83543

§ 371 Date: May 4, 2009

Related U.S. Application Data
(60) Provisional application No. 60/864,378, filed on Nov. 3, 2006.

(51) Int. Cl. A61K 31/506; C07D 401/14; C07D 413/14; C07D 405/14; A61K 31/5377; A61K 31/538; A61K 31/55; C12N 5/00; A61P 35/00; A61P 37/00; A61P 3/00; A61P 25/00; A61P 9/00; A61P 37/08

(52) U.S. Cl. 514/217.06; 544/331; 544/122; 544/105; 540/601; 514/275; 514/235.8; 514/230.5; 435/325

(57) ABSTRACT

The invention provides a novel class of compounds, pharmaceutical compositions comprising such compounds and methods of using such compounds to beat or prevent diseases or disorders associated with abnormal or deregulated kinase activity, particularly diseases or disorders that involve abnormal activation of c-kit, PDGFRα and PDGFRβ kinases. Formula (I).

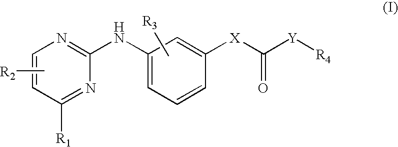

### CROSS-REFERENCE TO RELATED APPLICATIONS

This application claims the benefit of priority to U.S. Provisional Patent Application No. 60/864,378, filed 3 Nov. 2006. The full disclosure of this application is incorporated herein by reference in its entirety and for all purposes.

### BACKGROUND OF THE INVENTION

1. Field of the Invention

The invention provides a novel class of compounds, pharmaceutical compositions comprising such compounds and methods of using such compounds to treat or prevent diseases or disorders associated with abnormal or deregulated kinase activity, particularly diseases or disorders that involve abnormal activation of c-kit, PDGFRα and PDGFRβ kinases.

2. Background

The protein kinases represent a large family of proteins, which play a central role in the regulation of a wide variety of cellular processes and maintaining control over cellular function. A partial, non-limiting, list of these kinases include: receptor tyrosine kinases such as platelet-derived growth factor receptor kinase (PDGF-R), the nerve growth factor receptor, trkB, and the fibroblast growth factor receptor, FGFR3, B-RAF; non-receptor tyrosine kinases such Abl and the fusion kinase BCR-Abl, Lck, Bmx and c-src; and serine/threonine kinases such as c-RAF, sgk, MAP kinases (e.g., MKK4, MKK6, etc.) and SAPK2α and SAPK2β. Aberrant kinase activity has been observed in many disease states including benign and malignant proliferative disorders as well as diseases resulting from inappropriate activation of the immune and nervous systems.

The novel compounds of this invention inhibit the activity of one or more protein kinases and are, therefore, expected to be useful in the treatment of kinase-associated diseases.

### SUMMARY OF THE INVENTION

In one aspect, the present invention provides compounds of Formula I:

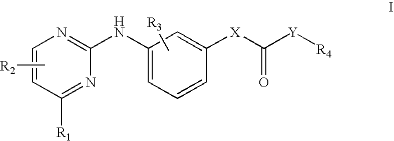

in which:

X is selected from a bond and NH;

Y is selected from a bond and NH;

R₁ is selected from cyclohexyl, pyridinyl, quinolinyl, isoquinolinyl and phenyl; wherein said cyclohexyl, pyridinyl, quinolinyl, isoquinolinyl or phenyl of R₁ can be optionally substituted with 1 to 3 radicals independently selected from halo, C₁₋₆alkyl, C₁₋₆alkoxy, halo-substituted-C₁₋₆alkyl, halo-substituted-C₁₋₆-alkoxy, —NR₅ₐR_{5b}, —OX₁NR₅ₐR_{5b} and heterocyclyl; wherein X₁ is independently selected from a bond and C₁₋₄alkylene; and R₅ₐ and R_{5b} are independently selected from hydrogen, C₁₋₆-alkyl, C₁₋₆-alkoxy, halo-substituted-C₁₋₆-alkyl and halo-substituted-C alkoxy;

R₂ is selected from halo, C₁₋₆alkyl, C₁₋₆alkoxy, halo-substituted-C₁₋₆alkyl and halo-substituted-C₁₋₆alkoxy;

R₃ is selected from halo, C₁₋₆alkyl, C₁₋₆alkoxy, halo-substituted-C₁₋₆alkyl and halo-substituted-C₁₋₆alkoxy;

R₄ is heteroaryl substituted with 1 to 3 radicals independently selected from halo, cyano, C₁₋₆alkyl, C₁₋₆alkoxy, halo-substituted-C₁₋₆alkyl, halo-substituted-C₁₋₆alkoxy, C₆₋₁₀aryl-C₀₋₄alkyl, heteroaryl, heterocyclyl, —X₁NR₅R₅, —X₁NR₅OR₅, —X₁NR₅X₁OR₅, —X₁NR₅X₁C(O)NR₅R₅, —X₁S(O)₂NR₅R₅, —X₁S(O)₂R₅, —X₁NR₅R₅, —X₁NR₅OR₅, —X₁C(O)R₅, —X₁₀X₂OR₅, —X₁R₅, —X₁R₅, —X₁C(O)OR₅, —X₁OR₅ and —X₁OX₁OR₅; wherein each X₁ is independently selected from a bond and C₁₋₄alkylene; X₂ is C₁₋₄alkylene; and each R₅ is independently selected from hydrogen, C₁₋₆alkyl, C₂₋₆alkenyl, C₃₋₁₂cycloalkyl, C₆₋₁₀aryl-C₀₋₄alkyl, heteroaryl-C₀₋₄alkyl and heterocyclyl;

wherein said aryl, cycloalkyl, heteroaryl or heterocyclyl substituents of R₄ can optionally be further substituted with 1 to 3 radicals independently selected from halo, hydroxy, cyano, C₁₋₆alkyl, C₁₋₆alkoxy, halo-substituted-C₁₋₆alkyl, halo-substituted-C₁₋₆alkoxy, -L-OR₆, -L-C(O)OR₆, -L-C(O)NR₆R₆ and -L-R₆; wherein L is selected from a bond and C₁₋₄alkylene; and R₆ is selected from hydrogen, C₁₋₆alkyl and heterocyclyl; with the proviso that R₄ is not pyridin-3-yl substituted by a trifluoromethyl radical; and the N-oxide derivatives, prodrug derivatives, protected derivatives, individual isomers and mixture of isomers thereof; and the pharmaceutically acceptable salts and solvates (e.g. hydrates) of such compounds.

In a second aspect, the present invention provides a pharmaceutical composition which contains a compound of Formula I or a N-oxide derivative, individual isomers and mixture of isomers thereof; or a pharmaceutically acceptable salt thereof, in admixture with one or more suitable excipients.

In a third aspect, the present invention provides a method of treating a disease in an animal in which inhibition of kinase activity, particularly c-kit, PDGFRα and/or PDGFRβ activity, can prevent, inhibit or ameliorate the pathology and/or symptomology of the diseases, which method comprises administering to the animal a therapeutically effective amount of a compound of Formula I or a N-oxide derivative, individual isomers and mixture of isomers thereof, or a pharmaceutically acceptable salt thereof.

In a fourth aspect, the present invention provides the use of a compound of Formula I in the manufacture of a medicament for treating a disease in an animal in which kinase activity, particularly c-kit, PDGFRα and/or PDGFRβ activity, contributes to the pathology and/or symptomology of the disease.

In a fifth aspect, the present invention provides a process for preparing compounds of Formula I and the N-oxide derivatives, prodrug derivatives, protected derivatives, individual isomers and mixture of isomers thereof, and the pharmaceutically acceptable salts thereof.

### DETAILED DESCRIPTION OF THE INVENTION

### Definitions

“Alkyl” as a group and as a structural element of other groups, for example halo-substituted-alkyl and alkoxy, can be either straight-chained or branched. C₁₋₄-alkoxy includes, methoxy, ethoxy, and the like. Halo-substituted alkyl includes trifluoromethyl, pentafluoroethyl, trifluoroethoxy (and the isomers thereof) and the like.

“Aryl” means a monocyclic or fused bicyclic aromatic ring assembly containing six to ten ring carbon atoms. For example, aryl may be phenyl or naphthyl, preferably phenyl. “Arylene” means a divalent radical derived from an aryl group.

“Heteroaryl” is a 5 to 10 member, unsaturated ring system containing 1 to 3 heteroatoms independently selected from —O—, —N═, —NR—, —C(O)—, —S—, —S(O)— or —S(O)₂—, wherein R is hydrogen, C₁₋₄alkyl or a nitrogen protecting group. Examples as used in this application include, but are not limited to, pyrazolyl, pyridinyl, indolyl, thiazolyl, 3-oxo-3,4-dihydro-2H-benzo[b][1,4]oxazin-6-yl, furanyl, benzo[b]furanyl, pyrrolyl, 1H-indazolyl, imidazo[1,2-a]pyridin-3-yl, oxazolyl, benzo[d]thiazol-6-yl, 1H-benzo[d][1,2,3]triazol-5-yl, quinolinyl, 1H-indolyl, 3,4-dihydro-2H-pyrano[2,3-b]pyridinyl and 2,3-dihydrofuro[2,3-b]pyridinyl, 3-oxo-3,4-dihydro-2H-benzo[b][1,4]oxazin-7-yl, etc.

“Cycloalkyl” means a saturated or partially unsaturated, monocyclic, fused bicyclic or bridged polycyclic ring assembly containing the number of ring atoms indicated. For example, C₃₋₁₀cycloalkyl includes cyclopropyl, cyclobutyl, cyclopentyl, cyclohexyl, etc.

“Heterocyclyl” means a 5 to 10 member, saturated or partially unsaturated ring system containing 1 to 3 heteroatoms independently selected from —O—, —N═, —NR—, —C(O)—, —S—, —S(O)— or —S(O)₂—, wherein R is hydrogen, C₁₋₄alkyl or a nitrogen protecting group. For example, heterocyclyl as used in this application to describe compounds of the invention includes morpholino, pyrrolidinyl, azepanyl, piperidinyl, isoquinolinyl, tetrahydrofuranyl, pyrrolidinyl, pyrrolidinyl-2-one, piperazinyl, piperidinylone, 1,4-dioxa-8-aza-spiro[4.5]dec-8-yl, 3,4-dihydroisoquinolin-2(1H)-yl, etc.

“Halogen” (or halo) preferably represents chloro or fluoro, but may also be bromo or iodo.

“Kinase Panel” is a list of kinases comprising Abl(human), Abl(T315I), JAK2, JAK3, ALK, JNK1α1, ALK4, KDR, Aurora-A, Lck, Blk, MAPK1, Bmx, MAPKAP-K2, BRK, MEK1, CaMKII(rat), Met, CDK1/cyclinB, p70S6K, CHK2, PAK2, CK1, PDGFRα, CK2, PDK1, c-kit, Pim-2, c-RAF, PKA(h), CSK, PKBA, cSrc, PKCα, DYRK2, Plk3, EGFR, ROCK-I, Fes, Ron, FGFR3, Ros, Flt3, SAPK2α, Fms, SGK, Fyn, SIK, GSK3β, Syk, IGF-1R, Tie-2, IKKβ-3, TrKB, IR, WNK3, IRAK4, ZAP-70, ITK, AMPK(rat), LIMK1, Rsk2, Axl, LKB1, SAPK2β, BrSK2, Lyn (h), SAPK3, BTK, MAPKAP-K3, SAPK4, CaMKIV, MARK1, Snk, CDK2/cyclinA, MINK, SRPK1, CDK3/cyclinE, MKK4(m), TAK1, CDK5/p25, MKK6(h), TBK1, CDK6/cyclinD3, MLCK, TrkA, CDK7/cyclinH/MAT1, MRCKβ, TSSK1, CHK1, MSK1, Yes, CK1d, MST2, ZIPK, c-Kit (D816V), MuSK, DAPK2, NEK2, DDR2, NEK6, DMPK, PAK-4, DRAK1, PAR-1Bα, EphA1, PDGFRβ, EphA2, Pim-1, EphA5, PKBβ, EphB2, PKCβI, EphB4, PKCε, FGFR1, PKCη, FGFR², PKCθ, FGFR4, PKD2, Fgr, PKG1β, Flt1, PRK2, Hck, PYK2, HIPK2, Ret, IKKα, RIPK2, IRR, ROCK-II(human), JNK2α2, Rse, JNK3, Rsk1(h), PI3 Ky, PI3 Kδ and PI3-Kβ. Compounds of the invention are screened against the kinase panel (wild type and/or mutation thereof) and inhibit the activity of at least one of said panel members.

“Mutant forms of BCR-Abl” means single or multiple amino acid changes from the wild-type sequence. Mutations in BCR-ABL act by disrupting critical contact points between protein and inhibitor (for example, Gleevec, and the like), more often, by inducing a transition from the inactive to the active state, i.e. to a conformation to which BCR-ABL and Gleevec is unable to bind. From analyses of clinical samples, the repertoire of mutations found in association with the resistant phenotype has been increasing slowly but inexorably over time. Mutations seem to cluster in four main regions. One group of mutations (G250E, Q252R, Y253F/H, E255KfV) includes amino acids that form the phosphate-binding loop for ATP (also known as the P-loop). A second group (V289A, F311L, T315I, F317L) can be found in the Gleevec binding site and interacts directly with the inhibitor via hydrogen bonds or Van der Waals' interactions. The third group of mutations (M351T, E355G) clusters in close proximity to the catalytic domain. The fourth group of mutations (H396R/P) is located in the activation loop, whose conformation is the molecular switch controlling kinase activation/inactivation. BCR-ABL point mutations associated with Gleevec resistance detected in CML and ALL patients include: M224V, L248V, G250E, G250R, Q252R, Q252H, Y253H, Y253F, E255K, E255V, D276G, T277A, V289A, F311L, T315I, T315N, F317L, M343T, M315T, E355G, F359V, F359A, V3791, F382L, L387M, L387F, H396P, H396R, A397P, S417Y, E459K, and F486S (Amino acid positions, indicated by the single letter code, are those for the GenBank sequence, accession number AAB60394, and correspond to ABL type 1a; Martinelli et al., Haematologica/The Hematology Journal, 2005, April; 90-4). Unless otherwise stated for this invention, Bcr-Abl refers to wild-type and mutant forms of the enzyme.

“Treat”, “treating” and “treatment” refer to a method of alleviating or abating a disease and/or its attendant symptoms.

### DESCRIPTION OF THE PREFERRED EMBODIMENTS

The c-kit gene encodes a receptor tyrosine kinase and the ligand for the c-kit receptor is called the stem cell factor (SCF), which is the principal growth factor for mast cells. The activity of the c-kit receptor protein tyrosine kinase is regulated in normal cells, and the normal functional activity of the c-kit gene product is essential for maintenance of normal hematopoeisis, melanogenesis, genetogenesis, and growth and differentiation of mast cells. Mutations that cause constitutive activation of c-kit kinase activity in the absence of SCF binding are implicated in various diseases ranging from asthma to malignant human cancers.

In one embodiment, with reference to compounds of Formula I, are compounds of Formula Ia:

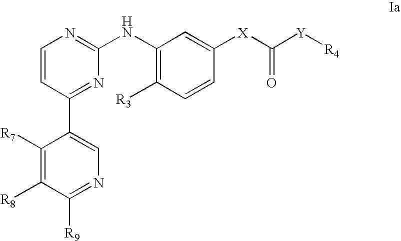

in which:

X is selected from a bond and NH;

y is selected from a bond and NH; wherein either X or Y, but not both, is a bond;

R₃ is selected from halo, methyl, methoxy, trifluoromethyl and trifluoromethoxy;

R₄ is heteroaryl substituted with 1 to 3 radicals independently selected from halo, cyano, C₁₋₆alkyl, C₁₋₆alkoxy, halo-substituted-C₁₋₆alkyl, halo-substituted-C₁₋₆alkoxy, C₆₋₁₀aryl-C₀₋₄alkyl, heteroaryl, heterocyclyl, —X₁NR₅R₅, —X₁NR₅OR₅, —X₁NR₅X₁OR₅, —X₁NR₅X₁C(O)NR₅R₅, —X₁S(O)₂NR₅R₅, —X₁S(O)₂R₅, —X₁NR₅R₅, —X₁NR₅OR₅, —X₁C(O)R₅, —X₁OX₂OR₅, —OX₁R₅, —X₁R₅, —X₁C(O)OR₅, —X₁OR₅ and —X₁OX₁OR₅; wherein each X₁ is independently selected from a bond and C₁₋₄alkylene; X₂ is C₁₋₄alkylene; and each R₅ is independently selected from hydrogen, C₁₋₆alkyl, C₂₋₆alkenyl, C₃₋₂cycloalkyl, C₆₋₁₀aryl-C₀₋₄alkyl, heteroaryl-C₀₋₄alkyl and heterocyclyl;

wherein said aryl, cycloalkyl, heteroaryl or heterocyclyl substituents of R₄ can optionally be further substituted with 1 to 3 radicals independently selected from halo, hydroxy, cyano, C₁₋₆alkyl, C₁₋₆alkoxy, halo-substituted-C₁₋₆alkyl, halo-substituted-C₁₋₆alkoxy, -L-OR₆, -L-C(O)OR₆, -L-C(O)NR₆R₆ and -L-R₆; wherein L is selected from a bond and C₁₋₄alkylene; and R₆ is selected from hydrogen, C₁₋₆alkyl and heterocyclyl;

R₇ is hydrogen;

R₅ is selected from hydrogen, halo, methoxy, amino, difluoromethoxy, trifluoromethyl, pyrrolidinyl, morpholino, 2-methyl-morpholino, 2,6-dimethyl-morpholino, cyano, —NR₅ₐR_{5b} and methyl; or R₇ and R₈ together with the carbon atoms to which R₇ and

R₈ are attached form phenyl; wherein R₅ₐ and R_{5b} are independently selected from hydrogen, C₁₋₆alkyl, C₁₋₆alkoxy, halo-substituted-C₁₋₆alkyl and halo-substituted-C₁₋₆alkoxy; R₉ is selected from hydrogen, morpholino, halo, C₁₋₆alkyl, C₁₋₆alkoxy, halo-substituted-C₁₋₆alkyl, halo-substituted-C₁₋₆alkoxy, —NR₅ₐR₅b, —OX₁NR₅ₐR_{5b} and heterocyclyl; wherein X₁ is independently selected from a bond and C₁₋₄alkylene; and R₅ₐ and R_{5b} are independently selected from hydrogen, C₁₋₆alkyl, C₁₋₆alkoxy, halo-substituted-C₁₋₆alkyl and halo-substituted-C₁₋₆alkoxy.

In another embodiment: R₃ is methyl; and R₄ is pyrazolyl, pyridinyl, indolyl, indolin-2-yl, thienyl, thiazolyl, 3-oxo-3,4-dihydro-2H-benzo[b][1,4]oxazin-6-yl, furanyl, benzo[b]furanyl, 1,3,4-thiadiazolyl, benzo[b]thiophenyl, pyrrolyl, 1H-indazolyl, imidazo[1,2-a]pyridin-3-yl, oxazolyl, benzo[d]thiazol-6-yl, 1H-benzo[d][1,2,3]triazol-5-yl, quinolinyl, 1H-indolyl, 3,4-dihydro-2H-pyrano[2,3-b]pyridinyl, 3-oxo-3,4-dihydro-2H-benzo[b][1,4]oxazin-7-yl and 2,3-dihydrofuro[2,3-b]pyridinyl;

wherein said heteroaryls of R₄ are substituted with 1 to 3 radicals independently selected from halo, hydroxy, cyano, methyl, amino, phenyl, hydroxy-ethyl(methyl)amino, piperidinyl, trifluoromethyl, 2-methylallyloxy, cyclopropyl-methyl(propyl)amino-methyl, trifluoromethoxy, 3,4-dihydroisoquinolin-2(1H)-yl, amino-carbonyl-methyl(ethyl)amino-methyl, pyridinyl-methyl(ethyl)-amino-methyl, isopropyl(ethyl)-amino-methyl, propyl(ethyl)-amino-methyl, morpholino, butyl(methyl)amino-methyl, isobutyl(methyl)amino-methyl, benzyl(ethyl)amino-methyl, pyridinyl, pyrrolidinyl, azepanyl, hydroxy-propyloxy, ethyl, methoxy, methyl-carbonyl, ethoxy, propyloxy, t-butyl, benzyl, propyl, isopropyloxy, isopropyl, diethylamino-sulfonyl, methyl-sulfonyl, isopropyl-sulfonyl, diethyl-amino-methyl, trifluoroethoxy, piperidinyl, isoquinolinyl, (hydroxy-ethyl)(methyl)amino, difluoro-ethoxy, cyclopropyl, cyclopropyl-methoxy and tetrahydrofuranyl-oxy;

wherein said aryl, cycloalkyl, heteroaryl or heterocyclyl substituents of R₄ can optionally be further substituted with 1 to 3 radicals independently selected from halo, methyl, pyrrolidinyl-methyl, trifluoromethyl, hydroxy-methyl, hydroxy and cyano.

In another embodiment, R₉ is selected from hydrogen and dimethyl-amino-propyloxy.

In another embodiment are compounds selected from: N-(3-(4-(pyridin-3-yl)pyrimidin-2-ylamino)-4-methylphenyl)-5-chloro-1H-indole-2-carboxamide; N-(3-(4-(pyridin-3-yl)pyrimidin-2-ylamino)-4-methylphenyl)-1-ethyl-3-methyl-1H-pyrazole-5-carboxamide; N-(3-(4-(pyridin-3-yl)pyrimidin-2-ylamino)-4-methylphenyl)-1,3-dimethyl-1H-pyrazole-5-carboxamide; N-(3-(4-(pyridin-3-yl)pyrimidin-2-ylamino)-4-methylphenyl)-5-(trifluoromethyl)-2-methyloxazole-4-carboxamide; N-(3-(4-(pyridin-3-yl)pyrimidin-2-ylamino)-4-methylphenyl)-2-morpholinopyridine-4-carboxamide; N-(3-(4-(pyridin-3-yl)pyrimidin-2-ylamino)-4-methylphenyl)-6-methoxypyridine-3-carboxamide; N-(3-(4-(pyridin-3-yl)pyrimidin-2-ylamino)-4-methylphenyl)-6-methoxypyridine-3-carboxamide; N-(3-(4-(pyridin-3-yl)pyrimidin-2-ylamino)-4-methylphenyl)-1,5-dimethyl-1H-pyrazole-3-carboxamide; N-(3-(4-(5-methylpyridin-3-yl)pyrimidin-2-ylamino)-4-methylphenyl)-1,5-dimethyl-1H-pyrazole-3-carboxamide; N-(3-(4-(5-methoxypyridin-3-yl)pyrimidin-2-ylamino)-4-methylphenyl)-1,5-dimethyl-1H-pyrazole-3-carboxamide; 2-(2,2-difluoroethoxy)-N-(3-(4-(pyridin-3-yl)pyrimidin-2-ylamino)-4-methylphenyl)pyridine-4-carboxamide; 6-(2,2,2-trifluoroethoxy)-N-(3-(4-(pyridin-3-yl)pyrimidin-2-ylamino)-4-methylphenyl)pyridine-3-carboxamide; 3-(4-(pyridin-3-yl)pyrimidin-2-ylamino)-N-(3,4-dihydro-3-oxo-2H-benzo[b][1,4]oxazin-6-yl)-4-methylbenzamide; and N-(3-(4-(5-methoxypyridin-3-yl)pyrimidin-2-ylamino)-4-methylphenyl)-1,5-dimethyl-1H-pyrazole-3-carboxamide.

A representative number of compounds of the invention are detailed in the Examples and Table 1, infra.

In one embodiment, the invention provides methods for treating a disease or condition modulated by the c-kit and PDGFRα/β kinase receptors, comprising administering compounds of Formula I, or pharmaceutically acceptable salts or pharmaceutical compositions thereof.

Examples of c-kit mediated disease or conditions which may be mediated using the compounds and compositions of the invention include but are not limited to a neoplastic disorder, an allergy disorder, an inflammatory disorder, an autoimmune disorder, a graft-versus-host disease, a Plasmodium related disease, a mast cell associated disease, a metabolic syndrome, a CNS related disorder, a neurodegenerative disorder, a pain condition, a substance abuse disorder, a prion disease, a cancer, a heart disease, a fibrotic disease, idiopathic arterial hypertension (IPAH), or primary pulmonary hypertension (PPH).

Examples of a plasmodium related disease which may be treated using compounds and compositions of the invention include but are not limited to malaria.

Examples of a mast cell associated disease which may be treated using compounds and compositions of the invention include but are not limited to acne and Propionibacterium acnes, Fibrodysplasia ossificans progressiva (FOP), inflammation and tissue destruction induced by exposure to chemical or biological weapons (such as anthrax and sulfur-mustard), Cystic fibrosis; renal disease, inflammatory muscle disorders, HIV, type II diabetes, cerebral ischemia, mastocytosis, drug dependence and withdrawal symptoms, CNS disorders, preventing and minimizing hair loss, bacterial infections, interstitial cystitis, inflammatory bowel diseases, tumor angiogenesis, autoimmune diseases, inflammatory diseases, Multiple Sclerosis (MS), allergic disorders (including asthma), irritable bowel syndrome (IBS), nasal polyposis, and bone loss.

Examples of neoplastic disorders which may be treated using the compounds and compositions of the invention include but are not limited to mastocytosis, gastrointestinal stromal tumor, small cell lung cancer, non-small cell lung cancer, acute myelocytic leukemia, acute lymphocytic leukemia, myelodyplastic syndrome, chronic myelogenous leukemia, colorectal carcinoma, gastric carcinoma, testicular cancer, glioblastoma or astrocytoma.

Examples of allergy disorders which may be treated using the compounds and compositions of the invention include but are not limited to asthma, allergic rhinitis, allergic sinusitis, anaphylactic syndrome, urticaria, angioedema, atopic dermatitis, allergic contact dermatitis, erythema nodosum, erythema multiforme, cutaneous necrotizing venulitis, insect bite skin inflammation, or blood sucking parasite infestation.

Examples of inflammatory disorders which may be treated using the compounds and compositions of the invention include but are not limited to rheumatoid arthritis, conjunctivitis, rheumatoid spondylitis, osteoarthritis or gouty arthritis.

Examples of autoimmune disorders which may be treated using the compounds and compositions of the invention include but are not limited to multiple sclerosis, psoriasis, intestine inflammatory disease, ulcerative colitis, Crohn's disease, rheumatoid arthritis, polyarthritis, local or systemic scleroderma, systemic lupus erythematosus, discoid lupus erythematosis, cutaneous lupus, dermatomyositis, polymyositis, Sjogren's syndrome, nodular panarteritis, autoimmune enteropathy or proliferative glomerulonephritis.

Examples of graft-versus-host diseases which may be treated using the compounds and compositions of the invention include but are not limited to organ transplantation graft rejection, such as kidney transplantation, pancreas transplantation, liver transplantation, heart transplantation, lung transplantation, or bone marrow transplantation.

Examples of metabolic syndrome which may be treated using the compounds and compositions of the invention include but are not limited to type I diabetes, type II diabetes, or obesity.

Examples of CNS related disorders which may be treated using the compounds and compositions of the invention include but are not limited to depression, dysthymic disorder, cyclothymic disorder, anorexia, bulimia, premenstrual syndrome, post-menopause syndrome, mental slowing, loss of concentration, pessimistic worry, agitation, self-deprecation and decreased libido, an anxiety disorder, a psychiatric disorder or schizophrenia.

Examples of depression conditions which may be treated using the compounds and compositions of the invention include but are not limited to bipolar depression, severe or melancholic depression, atypical depression, refractory depression, or seasonal depression. Examples of anxiety disorders which may be treated using the compounds and compositions of the invention include but are not limited to anxiety associated with hyperventilation and cardiac arrhythmias, phobic disorders, obsessive-compulsive disorder, posttraumatic stress disorder, acute stress disorder, and generalized anxiety disorder. Examples of psychiatric disorders which may be treated using the compounds and compositions of the invention include but are not limited to panic attacks, including psychosis, delusional disorders, conversion disorders, phobias, mania, delirium, dissociative episodes including dissociative amnesia, dissociative fugue and dissociative suicidal behavior, self-neglect, violent or aggressive behavior, trauma, borderline personality, and acute psychosis such as schizophrenia, including paranoid schizophrenia, disorganized schizophrenia, catatonic schizophrenia, and undifferentiated schizophrenia.

Examples of neurodegenerative disorder which may be treated using the compounds and compositions of the invention include but are not limited to Alzheimer's disease, Parkinson's disease, Huntington's disease, the prion diseases, Motor Neuron Disease (MND), or Amyotrophic Lateral Sclerosis (ALS).

Examples of pain conditions which may be treated using the compounds and compositions of the invention include but are not limited to acute pain, postoperative pain, chronic pain, nociceptive pain, cancer pain, neuropathic pain or psychogenic pain syndrome.

Examples of substance use disorders which may be treated using the compounds and compositions of the invention include but are not limited to drug addiction, drug abuse, drug habituation, drug dependence, withdrawal syndrome or overdose.

Examples of cancers which may be treated using the compounds and compositions of the invention include but are not limited to melanoma, gastrointestinal stromal tumor (GIST), small cell lung cancer, colorectal cancer or other solid tumors.

Examples of fibrotic diseases which may be treated using the compounds and compositions of the invention include but are not limited to hepatitis C(HCV), liver fibrosis, nonalcoholic steatohepatitis (NASH), cirrhosis in liver, pulmonary fibrosis, cardiac fibrosis, or bone marrow fibrosis.

In another embodiment, the invention provides methods for treating a disease or condition modulated by the c-kit kinase receptor, comprising administering compounds of Formula I, or pharmaceutically acceptable salts or pharmaceutical compositions thereof.

### Pharmacology and Utility

Compounds of the invention modulate the activity of kinases and, as such, are useful for treating diseases or disorders in which kinases, contribute to the pathology and/or symptomology of the disease. Examples of kinases that are inhibited by the compounds and compositions described herein and against which the methods described herein are useful include, but are not limited to c-kit, Abl, Lyn, MAPK14 (p38delta), PDGFRα, PDGFRβ, ARG, BCR-Abl, BRK, EphB, Fms, Fyn, KDR, LCK, b-Raf, c-Raf, SAPK2, Src, Tie2 and TrkB kinase.

Malaria is caused by protozoan parasites of the genus Plasmodium. Four species of Plasmodium can produce the disease in its various forms: Plasmodium falciparum; Plasmodium vivax; Plasmodium ovale; and Plasmodium malaria. P. falciparum, the most widespread and dangerous, can lead to fatal cerebral malaria if left untreated. Protein tyrosine kinase activity is distributed in all the stages of P. falciparum parasite maturation and kinase inhibitors of the present invention can be used for treating Plasmodium related diseases. Tyrosine kinase inhibitors of the present invention, in particular c-kit inhibitors can be a route for treating Plasmodium related diseases through inhibition of the growth of Plasmodium falciparum. The in vitro assay, infra, is used as a means to determine the activity of compounds of the invention against a variety of malarial parasite strains.

Mast cells (MC) are tissue elements derived from a particular subset of hematopoietic stem cells that produce a large variety of mediators most of which having strong pro-inflammatory activities. Since MCs are distributed in almost all the body sites, hypersecretion of mediators by activated elements can lead to multiple organ failures. Mast cells are, therefore, central players involved in many diseases. The present invention relates to a method for treating mast cell associated diseases comprising administering a compound capable of depleting mast cells or a compound inhibiting mast cell degranulation, to a human in need of such treatment. Such compounds can be chosen from c-kit inhibitors and more particularly non-toxic, selective and potent c-kit inhibitors. Preferably, said inhibitors are unable to promote death of IL-3 dependent cells cultured in presence of IL-3.

Mast cell associated diseases include, but are not limited to: acne and Propionibacterium acnes (acne encompasses all forms of chronic inflammation of the skin including those induced by Propionibacterium acnes); an extremely rare and disabling genetic disorder of connective tissue known as Fibrodysplasia ossificans progressiva (FOP); the detrimental effects of inflammation and tissue destruction induced by exposure to chemical or biological weapons (such as anthrax, sulfur-mustard, etc.); Cystic fibrosis (a lung, digestive and reproductive systems genetic disease); renal disease such as Acute nephritic syndrome, glomerulonephritis, renal amyloidosis, renal interstitial fibrosis (the final common pathway leading to end-stage renal disease in various nephropathies); inflammatory muscle disorders including myositis and muscular dystrophy; HIV (for example, depleting HIV infected mast cells can be a new route for treating HIV infection and related diseases); treating type II diabetes, obesity and related disorders (mast cells regulate a number of the processes that contribute to the development of atherosclerosis, including hyperglycemia, hypercholesterolemia, hypertension, endothelial dysfunction, insulin resistance, and vascular remodeling; cerebral ischemia; mastocytosis (a very heterogeneous group of disorders characterized by an abnormal accumulation of mast cells in different tissues, mainly in the skin and the bone marrow, but also in spleen, liver, lymph nodes, and the gastrointestinal tract); drug dependence and withdrawal symptoms (particularly drug addiction, drug abuse, drug habituation, drug dependence, withdrawal syndrome and overdose); CNS disorders (particularly depression, schizophrenia, anxiety, migraine, memory loss, pain and neurodegenerative diseases); promoting hair growth (including preventing and minimizing hair loss); bacterial infections (particularly infections caused by FimH expressing bacteria); interstitial cystitis (a chronic inflammation of the bladder wall resulting in tissue damage, especially at the interstices between the cells in the lining of the bladder); Inflammatory bowel diseases (generally applied to four diseases of the bowel, namely Crohn's disease, ulcerative colitis, indeterminate colitis, and infectious colitis); tumor angiogenesis; autoimmune diseases (particularly multiple sclerosis, ulcerative colitis, Crohn's disease, rheumatoid arthritis and polyarthritis, scleroderma, lupus erythematosus, dermatomyositis, pemphigus, polymyositis, vasculitis and graft-versus host diseases); inflammatory diseases such as rheumatoid arthritis (RA); Multiple Sclerosis (MS); allergic disorders (particularly allergic rhinitis, allergic sinusitis, anaphylactic syndrome, urticaria, angioedema, atopic dermatitis, allergic contact dermatitis, erythema nodosum, erythema multiforme, cutaneous necrotizing venulitis and insect bite skin inflammation, bronchial asthma); and bone loss.

Abelson tyrosine kinase (i.e. Abl, c-Abl) is involved in the regulation of the cell cycle, in the cellular response to genotoxic stress, and in the transmission of information about the cellular environment through integrin signaling. Overall, it appears that the Abl protein serves a complex role as a cellular module that integrates signals from various extracellular and intracellular sources and that influences decisions in regard to cell cycle and apoptosis. Abelson tyrosine kinase includes sub-types derivatives such as the chimeric fusion (oncoprotein) BCR-Abl with deregulated tyrosine kinase activity or the v-Abl. BCR-Abl is critical in the pathogenesis of 95% of chronic myelogenous leukemia (CML) and 10% of acute lymphocytic leukemia. STI-571 (Gleevec) is an inhibitor of the oncogenic BCR-Abl tyrosine kinase and is used for the treatment of chronic myeloid leukemia (CML). However, some patients in the blast crisis stage of CML are resistant to STI-571 due to mutations in the BCR-Abl kinase. Over 22 mutations have been reported to date with the most common being G250E, E255V, T3151, F317L and M351T.

Some compounds of the present invention inhibit abl kinase, especially v-abl kinase. Some of the compounds of the present invention also inhibit wild-type BCR-Abl kinase and mutations of BCR-Abl kinase and are thus suitable for the treatment of Bcr-abl-positive cancer and tumor diseases, such as leukemias (especially chronic myeloid leukemia and acute lymphoblastic leukemia, where especially apoptotic mechanisms of action are found), and also shows effects on the subgroup of leukemic stem cells as well as potential for the purification of these cells in vitro after removal of said cells (for example, bone marrow removal) and reimplantation of the cells once they have been cleared of cancer cells (for example, reimplantation of purified bone marrow cells).

The Ras-Raf-MEK-ERK signaling pathway mediates cellular response to growth signals. Ras is mutated to an oncogenic form in ˜15% of human cancer. The Raf family belongs to the serine/threonine protein kinase and it includes three members, A-Raf, B-Raf and c-Raf (or Raf-1). The focus on Raf being a drug target has centered on the relationship of Raf as a downstream effector of Ras. However, recent data suggests that B-Raf may have a prominent role in the formation of certain tumors with no requirement for an activated Ras allele (Nature 417, 949-954 (1 Jul. 2002). In particular, B-Raf mutations have been detected in a large percentage of malignant melanomas.

Existing medical treatments for melanoma are limited in their effectiveness, especially for late stage melanomas. The compounds of the present invention also inhibit cellular processes involving b-Raf kinase, providing a new therapeutic opportunity for treatment of human cancers, especially for melanoma.

The compounds of the present invention also inhibit cellular processes involving c-Raf kinase. c-Raf is activated by the ras oncogene, which is mutated in a wide number of human cancers. Therefore inhibition of the kinase activity of c-Raf may provide a way to prevent ras mediated tumor growth [Campbell, S. L., Oncogene, 17, 1395 (1998)].

PDGF (Platelet-derived Growth Factor) is a very commonly occurring growth factor, which plays an important role both in normal growth and also in pathological cell proliferation, such as is seen in carcinogenesis and in diseases of the smooth-muscle cells of blood vessels, for example in atherosclerosis and thrombosis. Compounds of the invention can inhibit PDGF receptor (PDGFR) activity and are, therefore, suitable for the treatment of: tumor diseases, such as gliomas, sarcomas, prostate tumors, and tumors of the colon, breast, and ovary; hypereosinophilia; fibrosis; pulmonary hypertension; and cardiovascular diseases.

Compounds of the present invention, can be used not only as a tumor-inhibiting substance, for example in small cell lung cancer, but also as an agent to treat non-malignant proliferative disorders, such as atherosclerosis, thrombosis, psoriasis, scleroderma and fibrosis, as well as for the protection of stem cells, for example to combat the hemotoxic effect of chemotherapeutic agents, such as 5-fluoruracil, and in asthma. Compounds of the invention can especially be used for the treatment of diseases, which respond to an inhibition of the PDGF receptor kinase.

Compounds of the present invention show useful effects in the treatment of disorders arising as a result of transplantation, for example, allogenic transplantation, especially tissue rejection, such as especially obliterative bronchiolitis (OB), i.e. a chronic rejection of allogenic lung transplants. In contrast to patients without OB, those with OB often show an elevated PDGF concentration in bronchoalveolar lavage fluids.

Compounds of the present invention are also effective in diseases associated with vascular smooth-muscle cell migration and proliferation (where PDGF and PDGF-R often also play a role), such as restenosis and atherosclerosis. These effects and the consequences thereof for the proliferation or migration of vascular smooth-muscle cells in vitro and in vivo can be demonstrated by administration of the compounds of the present invention, and also by investigating its effect on the thickening of the vascular intima following mechanical injury in vivo.

The trk family of neurotrophin receptors (trkA, trkb, trkC) promotes the survival, growth and differentiation of the neuronal and non-neuronal tissues. The TrkB protein is expressed in neuroendocrine-type cells in the small intestine and colon, in the alpha cells of the pancreas, in the monocytes and macrophages of the lymph nodes and of the spleen, and in the granular layers of the epidermis (Shibayama and Koizumi, 1996). Expression of the TrkB protein has been associated with an unfavorable progression of Wilms tumors and of neuroblastomas. TkrB is, moreover, expressed in cancerous prostate cells but not in normal cells. The signaling pathway downstream of the trk receptors involves the cascade of MAPK activation through the Shc, activated Ras, ERK-1 and ERK-2 genes, and the PLC-gammal transduction pathway (Sugimoto et al., 2001).

The kinase, c-Src transmits oncogenic signals of many receptors. For example, over-expression of EGFR or HER2/neu in tumors leads to the constitutive activation of c-src, which is characteristic for the malignant cell but absent from the normal cell. On the other hand, mice deficient in the expression of c-src exhibit an osteopetrotic phenotype, indicating a key participation of c-src in osteoclast function and a possible involvement in related disorders.

The Tec family kinase, Bmx, a non-receptor protein-tyrosine kinase, controls the proliferation of mammary epithelial cancer cells.

Fibroblast growth factor receptor 3 was shown to exert a negative regulatory effect on bone growth and an inhibition of chondrocyte proliferation. Thanatophoric dysplasia is caused by different mutations in fibroblast growth factor receptor 3, and one mutation, TDII FGFR3, has a constitutive tyrosine kinase activity which activates the transcription factor Stat1, leading to expression of a cell-cycle inhibitor, growth arrest and abnormal bone development (Su et al., Nature, 1997, 386, 288-292). FGFR3 is also often expressed in multiple myeloma-type cancers. Inhibitors of FGFR3 activity are useful in the treatment of T-cell mediated inflammatory or autoimmune diseases including but not limited to rheumatoid arthritis (RA), collagen II arthritis, multiple sclerosis (MS), systemic lupus erythematosus (SLE), psoriasis, juvenile onset diabetes, Sjogren's disease, thyroid disease, sarcoidosis, autoimmune uveitis, inflammatory bowel disease (Crohn's and ulcerative colitis), celiac disease and myasthenia gravis.

The activity of serum and glucocorticoid-regulated kinase (SGK), is correlated to perturbed ion-channel activities, in particular, those of sodium and/or potassium channels and compounds of the invention can be useful for treating hypertension.

Lin et al (1997) J. Clin. Invest. 100, 8: 2072-2078 and P. Lin (1998) PNAS 95, 8829-8834, have shown an inhibition of tumor growth and vascularization and also a decrease in lung metastases during adenoviral infections or during injections of the extracellular domain of Tie-2 (Tek) in breast tumor and melanoma xenograft models. Tie2 inhibitors can be used in situations where neovascularization takes place inappropriately (i.e. in diabetic retinopathy, chronic inflammation, psoriasis, Kaposi's sarcoma, chronic neovascularization due to macular degeneration, rheumatoid arthritis, infantile haemangioma and cancers).

Lck plays a role in T-cell signaling. Mice that lack the Lck gene have a poor ability to develop thymocytes. The function of Lck as a positive activator of T-cell signaling suggests that Lck inhibitors may be useful for treating autoimmune disease such as rheumatoid arthritis.

JNKs, along with other MAPKs, have been implicated in having a role in mediating cellular response to cancer, thrombin-induced platelet aggregation, immunodeficiency disorders, autoimmune diseases, cell death, allergies, osteoporosis and heart disease. The therapeutic targets related to activation of the JNK pathway include chronic myelogenous leukemia (CML), rheumatoid arthritis, asthma, osteoarthritis, ischemia, cancer and neurodegenerative diseases. As a result of the importance of JNK activation associated with liver disease or episodes of hepatic ischemia, compounds of the invention may also be useful to treat various hepatic disorders. A role for JNK in cardiovascular disease such as myocardial infarction or congestive heart failure has also been reported as it has been shown JNK mediates hypertrophic responses to various forms of cardiac stress. It has been demonstrated that the JNK cascade also plays a role in T-cell activation, including activation of the IL-2 promoter. Thus, inhibitors of JNK may have therapeutic value in altering pathologic immune responses. A role for JNK activation in various cancers has also been established, suggesting the potential use of JNK inhibitors in cancer. For example, constitutively activated JNK is associated with HTLV-1 mediated tumorigenesis [Oncogene 13:135-42 (1996)]. JNK may play a role in Kaposi's sarcoma (KS). Other proliferative effects of other cytokines implicated in KS proliferation, such as vascular endothelial growth factor (VEGF), IL-6 and TNFα, may also be mediated by JNK. In addition, regulation of the c-jun gene in p210 BCR-ABL transformed cells corresponds with activity of JNK, suggesting a role for JNK inhibitors in the treatment for chronic myelogenous leukemia (CML) [Blood 92:2450-60 (1998)].

Certain abnormal proliferative conditions are believed to be associated with raf expression and are, therefore, believed to be responsive to inhibition of raf expression. Abnormally high levels of expression of the raf protein are also implicated in transformation and abnormal cell proliferation. These abnormal proliferative conditions are also believed to be responsive to inhibition of raf expression. For example, expression of the c-raf protein is believed to play a role in abnormal cell proliferation since it has been reported that 60% of all lung carcinoma cell lines express unusually high levels of c-raf mRNA and protein. Further examples of abnormal proliferative conditions are hyper-proliferative disorders such as cancers, tumors, hyperplasia, pulmonary fibrosis, angiogenesis, psoriasis, atherosclerosis and smooth muscle cell proliferation in the blood vessels, such as stenosis or restenosis following angioplasty. The cellular signaling pathway of which raf is a part has also been implicated in inflammatory disorders characterized by T-cell proliferation (T-cell activation and growth), such as tissue graft rejection, endotoxin shock, and glomerular nephritis, for example.

The stress activated protein kinases (SAPKS) are a family of protein kinases that represent the penultimate step in signal transduction pathways that result in activation of the c-jun transcription factor and expression of genes regulated by c-jun. In particular, c-jun is involved in the transcription of genes that encode proteins involved in the repair of DNA that is damaged due to genotoxic insults. Therefore, agents that inhibit SAPK activity in a cell prevent DNA repair and sensitize the cell to agents that induce DNA damage or inhibit DNA synthesis and induce apoptosis of a cell or that inhibit cell proliferation.

Mitogen-activated protein kinases (MAPKS) are members of conserved signal transduction pathways that activate transcription factors, translation factors and other target molecules in response to a variety of extracellular signals. MAPKs are activated by phosphorylation at a dual phosphorylation motif having the sequence Thr-X-Tyr by mitogen-activated protein kinase kinases (MKKs). In higher eukaryotes, the physiological role of MAPK signaling has been correlated with cellular events such as proliferation, oncogenesis, development and differentiation. Accordingly, the ability to regulate signal transduction via these pathways (particularly via MKK4 and MKK6) could lead to the development of treatments and preventive therapies for human diseases associated with MAPK signaling, such as inflammatory diseases, autoimmune diseases and cancer.

The family of human ribosomal S6 protein kinases consists of at least 8 members (RSK1, RSK2, RSK3, RSK4, MSK1, MSK2, p70S6K and p70S6 Kb). Ribosomal protein S6 protein kinases play important pleotropic functions, among them is a key role in the regulation of mRNA translation during protein biosynthesis (Eur. J. Biochem 2000 November; 267(21): 6321-30, Exp Cell Res. Nov. 25, 1999; 253 (1):100-9, Mol Cell Endocrinol. May 25, 1999; 151(1-2):65-77). The phosphorylation of the S6 ribosomal protein by p70S6 has also been implicated in the regulation of cell motility (Immunol. Cell Biol. 2000 August; 78(4):447-51) and cell growth (Prog. Nucleic Acid Res. Mol. Biol., 2000; 65:101-27), and hence, may be important in tumor metastasis, the immune response and tissue repair as well as other disease conditions.

The SAPK's (also called “jun N-terminal kinases” or “JNK's”) are a family of protein kinases that represent the penultimate step in signal transduction pathways that result in activation of the c-jun transcription factor and expression of genes regulated by c-jun. In particular, c-jun is involved in the transcription of genes that encode proteins involved in the repair of DNA that is damaged due to genotoxic insults. Agents that inhibit SAPK activity in a cell prevent DNA repair and sensitize the cell to those cancer therapeutic modalities that act by inducing DNA damage.

BTK plays a role in autoimmune and/or inflammatory disease such as systemic lupus erythematosus (SLE), rheumatoid arthritis, multiple vasculitides, idiopathic thrombocytopenic purpura (ITP), myasthenia gravis, and asthma. Because of BTK's role in B-cell activation, inhibitors of BTK are useful as inhibitors of B-cell mediated pathogenic activity, such as autoantibody production, and are useful for the treatment of B-cell lymphoma and leukemia.

CHK2 is a member of the checkpoint kinase family of serine/threonine protein kinases and is involved in a mechanism used for surveillance of DNA damage, such as damage caused by environmental mutagens and endogenous reactive oxygen species. As a result, it is implicated as a tumor suppressor and target for cancer therapy.

CSK influences the metastatic potential of cancer cells, particularly colon cancer.

Fes is a non-receptor protein tyrosine kinase that has been implicated in a variety of cytokine signal transduction pathways, as well as differentiation of myeloid cells. Fes is also a key component of the granulocyte differentiation machinery.

Flt3 receptor tyrosine kinase activity is implicated in leukemias and myelodysplastic syndrome. In approximately 25% of AML the leukemia cells express a constitutively active form of auto-phosphorylated (p) FLT3 tyrosine kinase on the cell surface. The activity of p-FLT3 confers growth and survival advantage on the leukemic cells. Patients with acute leukemia, whose leukemia cells express p-FLT3 kinase activity, have a poor overall clinical outcome. Inhibition of p-FLT3 kinase activity induces apoptosis (programmed cell death) of the leukemic cells.

Inhibitors of IKKα and IKKβ (1 & 2) are therapeutics for diseases which include rheumatoid arthritis, transplant rejection, inflammatory bowel disease, osteoarthritis, asthma, chronic obstructive pulmonary disease, atherosclerosis, psoriasis, multiple sclerosis, stroke, systemic lupus erythematosus, Alzheimer's disease, brain ischemia, traumatic brain injury, Parkinson's disease, amyotrophic lateral sclerosis, subarachnoid hemorrhage or other diseases or disorders associated with excessive production of inflammatory mediators in the brain and central nervous system.

Met is associated with most types of the major human cancers and expression is often correlated with poor prognosis and metastasis. Inhibitors of Met are therapeutics for diseases which include cancers such as lung cancer, NSCLC (non small cell lung cancer), bone cancer, pancreatic cancer, skin cancer, cancer of the head and neck, cutaneous or intraocular melanoma, uterine cancer, ovarian cancer, rectal cancer, cancer of the anal region, stomach cancer, colon cancer, breast cancer, gynecologic tumors (e.g., uterine sarcomas, carcinoma of the fallopian tubes, carcinoma of the endometrium, carcinoma of the cervix, carcinoma of the vagina or carcinoma of the vulva), Hodgkin's Disease, cancer of the esophagus, cancer of the small intestine, cancer of the endocrine system (e.g., cancer of the thyroid, parathyroid or adrenal glands), sarcomas of soft tissues, cancer of the urethra, cancer of the penis, prostate cancer, chronic or acute leukemia, solid tumors of childhood, lymphocytic lymphomas, cancer of the bladder, cancer of the kidney or ureter (e.g., renal cell carcinoma, carcinoma of the renal pelvis), pediatric malignancy, neoplasms of the central nervous system (e.g., primary CNS lymphoma, spinal axis tumors, brain stem glioma or pituitary adenomas), cancers of the blood such as acute myeloid leukemia, chronic myeloid leukemia, etc, Barrett's esophagus (pre-malignant syndrome) neoplastic cutaneous disease, psoriasis, mycoses fungoides and benign prostatic hypertrophy, diabetes related diseases such as diabetic retinopathy, retinal ischemia and retinal neovascularization, hepatic cirrhosis, cardiovascular disease such as atherosclerosis, immunological disease such as autoimmune disease and renal disease. Preferably, the disease is cancer such as acute myeloid leukemia and colorectal cancer.

The Nima-related kinase 2 (Nek2) is a cell cycle-regulated protein kinase with maximal activity at the onset of mitosis that localizes to the centrosome. Functional studies have implicated Nek2 in regulation of centrosome separation and spindle formation. Nek2 protein is elevated 2- to 5-fold in cell lines derived from a range of human tumors including those of cervical, ovarian, prostate, and particularly breast.

p70S6K-mediated diseases or conditions include, but are not limited to, proliferative disorders, such as cancer and tuberous sclerosis.

In accordance with the foregoing, the present invention further provides a method for preventing or treating any of the diseases or disorders described above in a subject in need of such treatment, which method comprises administering to said subject a therapeutically effective amount (See, “Administration and Pharmaceutical Compositions”, infra) of a compound of Formula I or a pharmaceutically acceptable salt thereof. For any of the above uses, the required dosage will vary depending on the mode of administration, the particular condition to be treated and the effect desired.

### Administration and Pharmaceutical Compositions

In general, compounds of the invention will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. In general, satisfactory results are indicated to be obtained systemically at daily dosages of from about 0.03 to 2.5 mg/kg per body weight. An indicated daily dosage in the larger mammal, e.g. humans, is in the range from about 0.5 mg to about 100 mg, conveniently administered, e.g. in divided doses up to four times a day or in retard form. Suitable unit dosage forms for oral administration comprise from ca. 1 to 50 mg active ingredient.

Compounds of the invention can be administered as pharmaceutical compositions by any conventional route, in particular enterally, e.g., orally, e.g., in the form of tablets or capsules, or parenterally, e.g., in the form of injectable solutions or suspensions, topically, e.g., in the form of lotions, gels, ointments or creams, or in a nasal, inhaled or suppository form. Pharmaceutical compositions comprising a compound of the present invention in free form or in a pharmaceutically acceptable salt form in association with at least one pharmaceutically acceptable carrier or diluent can be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Suitable formulations for transdermal applications include an effective amount of a compound of the present invention with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used. Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

Compounds of the invention can be administered in therapeutically effective amounts in combination with one or more therapeutic agents (pharmaceutical combinations). For example, synergistic effects can occur with other asthma therapies, for example, steroids and leukotriene antagonists.

For example, synergistic effects can occur with other immunomodulatory or anti-inflammatory substances, for example when used in combination with cyclosporin, rapamycin, or ascomycin, or immunosuppressant analogues thereof, for example cyclosporin A (CsA), cyclosporin G, FK-506, rapamycin, or comparable compounds, corticosteroids, cyclophosphamide, azathioprine, methotrexate, brequinar, leflunomide, mizoribine, mycophenolic acid, mycophenolate mofetil, 15-deoxyspergualin, immunosuppressant antibodies, especially monoclonal antibodies for leukocyte receptors, for example MHC, CD2, CD3, CD4, CD7, CD25, CD28, B7, CD45, CD58 or their ligands, or other immunomodulatory compounds, such as CTLA41g. Where the compounds of the invention are administered in conjunction with other therapies, dosages of the co-administered compounds will of course vary depending on the type of co-drug employed, on the specific drug employed, on the condition being treated and so forth.

The invention also provides for a pharmaceutical combinations, e.g. a kit, comprising a) a first agent which is a compound of the invention as disclosed herein, in free form or in pharmaceutically acceptable salt form, and b) at least one co-agent. The kit can comprise instructions for its administration.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the active ingredients, e.g. a compound of Formula I and a co-agent, are both administered to a patient simultaneously in the form of a single entity or dosage. The term “non-fixed combination” means that the active ingredients, e.g. a compound of Formula I and a co-agent, are both administered to a patient as separate entities either simultaneously, concurrently or sequentially with no specific time limits, wherein such administration provides therapeutically effective levels of the 2 compounds in the body of the patient. The latter also applies to cocktail therapy, e.g. the administration of 3 or more active ingredients.

### Processes for Making Compounds of the Invention

The present invention also includes processes for the preparation of compounds of the invention. In the reactions described, it can be necessary to protect reactive functional groups, for example hydroxy, amino, imino, thio or carboxy groups, where these are desired in the final product, to avoid their unwanted participation in the reactions. Conventional protecting groups can be used in accordance with standard practice, for example, see T. W. Greene and P. G. M. Wuts in “Protective Groups in Organic Chemistry”, John Wiley and Sons, 1991.

Compounds of Formula I, wherein Y is a bond and X is NH, can be prepared by proceeding as in the following Reaction Schemes I:

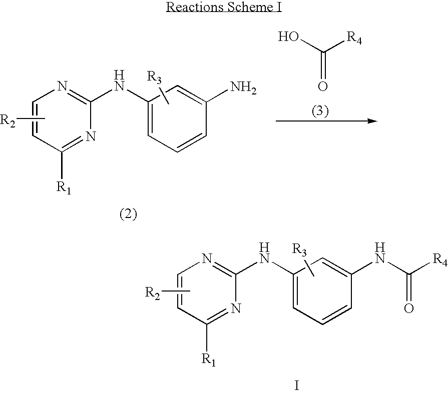

wherein R₁, R₂, R₃ and R₄ are as described in the Summary of the Invention. A compound of Formula I can be prepared by reacting of a compound of formula 2 with a compound of formula 3 in the presence of a suitable solvent (for example, DMF, and the like), a suitable coupling agent (for example, HATU, and the like) and a suitable base (for example, DIEA, and the like). The reaction is carried out in a temperature range of about 0° C. to about 60° C. and can take up to 24 hours to complete.

Compounds of Formula I, wherein X is a bond and Y is NH, can be prepared by proceeding as in the following Reaction Schemes II:

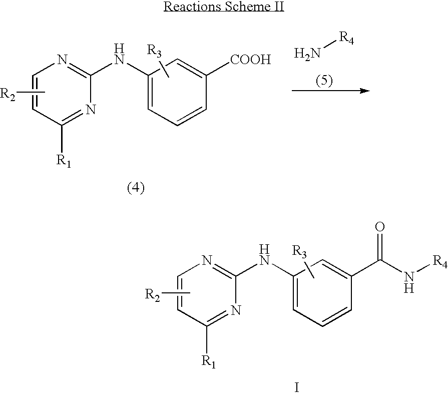

wherein R₁, R₂, R₃ and R₄ are as described in the Summary of the Invention. A compound of Formula I can be prepared by reacting of a compound of formula 4 with a compound of formula 5 in the presence of a suitable solvent (for example, DMF, and the like), a suitable coupling agent (for example, HATU, and the like) and a suitable base (for example, DIEA, and the like). The reaction is carried out in a temperature range of about 0° C. to about 60° C. and can take up to 24 hours to complete.

Detailed examples of the synthesis of compounds of formula I can be found in the Examples, infra.

### Additional Processes for Making Compounds of the Invention

A compound of the invention can be prepared as a pharmaceutically acceptable acid addition salt by reacting the free base form of the compound with a pharmaceutically acceptable inorganic or organic acid. Alternatively, a pharmaceutically acceptable base addition salt of a compound of the invention can be prepared by reacting the free acid form of the compound with a pharmaceutically acceptable inorganic or organic base. Alternatively, the salt forms of the compounds of the invention can be prepared using salts of the starting materials or intermediates.

The free acid or free base forms of the compounds of the invention can be prepared from the corresponding base addition salt or acid addition salt from, respectively. For example a compound of the invention in an acid addition salt form can be converted to the corresponding free base by treating with a suitable base (e.g., ammonium hydroxide solution, sodium hydroxide, and the like). A compound of the invention in a base addition salt form can be converted to the corresponding free acid by treating with a suitable acid (e.g., hydrochloric acid, etc.).

Compounds of the invention in unoxidized form can be prepared from N-oxides of compounds of the invention by treating with a reducing agent (e.g., sulfur, sulfur dioxide, triphenyl phosphine, lithium borohydride, sodium borohydride, phosphorus trichloride, tribromide, or the like) in a suitable inert organic solvent (e.g. acetonitrile, ethanol, aqueous dioxane, or the like) at 0 to 80° C.

Prodrug derivatives of the compounds of the invention can be prepared by methods known to those of ordinary skill in the art (e.g., for further details see Saulnier et al., (1994), Bioorganic and Medicinal Chemistry Letters, Vol. 4, p. 1985). For example, appropriate prodrugs can be prepared by reacting a non-derivatized compound of the invention with a suitable carbamylating agent (e.g., 1,1-acyloxyalkylcarbanochloridate, para-nitrophenyl carbonate, or the like).

Protected derivatives of the compounds of the invention can be made by means known to those of ordinary skill in the art. A detailed description of techniques applicable to the creation of protecting groups and their removal can be found in T. W. Greene, “Protecting Groups in Organic Chemistry”, 3^{rd} edition, John Wiley and Sons, Inc., 1999.

Compounds of the present invention can be conveniently prepared, or formed during the process of the invention, as solvates (e.g., hydrates). Hydrates of compounds of the present invention can be conveniently prepared by recrystallization from an aqueous/organic solvent mixture, using organic solvents such as dioxin, tetrahydrofuran or methanol.

Compounds of the invention can be prepared as their individual stereoisomers by reacting a racemic mixture of the compound with an optically active resolving agent to form a pair of diastereoisomeric compounds, separating the diastereomers and recovering the optically pure enantiomers. While resolution of enantiomers can be carried out using covalent diastereomeric derivatives of the compounds of the invention, dissociable complexes are preferred (e.g., crystalline diastereomeric salts). Diastereomers have distinct physical properties (e.g., melting points, boiling points, solubilities, reactivity, etc.) and can be readily separated by taking advantage of these dissimilarities. The diastereomers can be separated by chromatography, or preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques, Andre Collet, Samuel H. Wilen, “Enantiomers, Racemates and Resolutions”, John Wiley And Sons, Inc., 1981.

In summary, the compounds of Formula I can be made by a process, which involves:

(a) those of reaction schemes I and II, and

(b) optionally converting a compound of the invention into a pharmaceutically acceptable salt;

(c) optionally converting a salt form of a compound of the invention to a non-salt form;

(d) optionally converting an unoxidized form of a compound of the invention into a pharmaceutically acceptable N-oxide;

(e) optionally converting an N-oxide form of a compound of the invention to its unoxidized form;

(f) optionally resolving an individual isomer of a compound of the invention from a mixture of isomers;

(g) optionally converting a non-derivatized compound of the invention into a pharmaceutically acceptable prodrug derivative; and

(h) optionally converting a prodrug derivative of a compound of the invention to its non-derivatized form.

Insofar as the production of the starting materials is not particularly described, the compounds are known or can be prepared analogously to methods known in the art or as disclosed in the Examples hereinafter.

One of skill in the art will appreciate that the above transformations are only representative of methods for preparation of the compounds of the present invention, and that other well known methods can similarly be used.

### EXAMPLES

The present invention is further exemplified, but not limited, by the following examples that illustrate the preparation of compounds of Formula I according to the invention.

### Preparation of Intermediates

### Synthesis of 6-methyl-N1-(4-(pyridin-3-yl)pyrimidin-2-yl)benzene-1,3-diamine 5

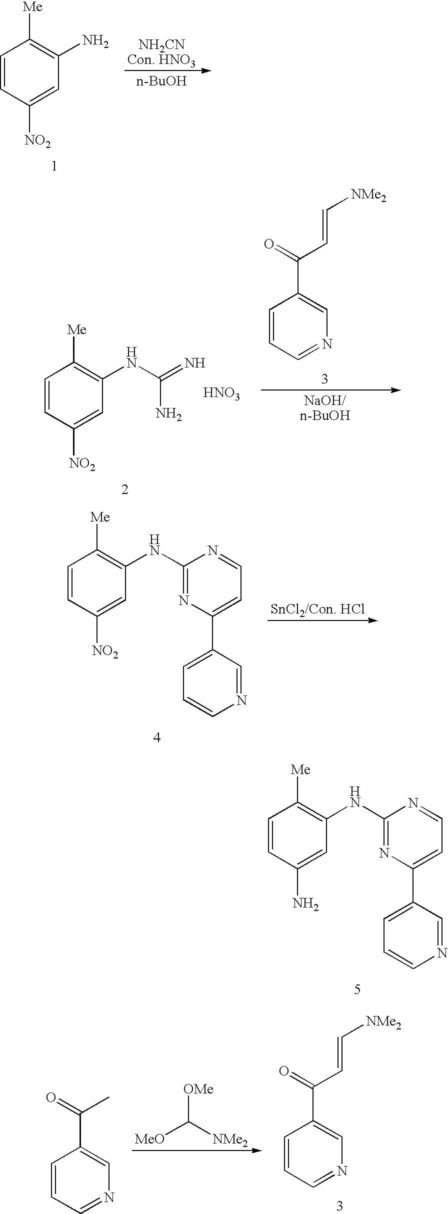

To 2-amino-4-nitro toluene 1 (0.033 mol) in n-butanol (29 mL) is added 2.1 g of 65% aq. nitric acid to form the nitrate salt followed by condensation with cyanamide (0.047 mmol) in water (2 mL). The resulting mixture is heated at reflux for 25 h. After cooling to 0° C., the precipitate is collected by filtration and washed with ethanol/diethyl:ether (1:1 v/v, 30 mL) to afford 2-methyl-5-nitrophenyl guanidine nitrate 2.

To 2-methyl-5-nitrophenyl guanidine 2 (0.0074 mol) in n-butanol (15 mL) is added 3 (0.0074 mol) and sodium hydroxide flakes (0.008 mol). The resulting mixture is heated at reflux for 12 h. After cooling to 0° C., the precipitate is collected by filtration and washed with isopropanol (6 mL) and methanol (3 mL) to afford 4. ¹HNMR (400 MHz, d₆-DMSO) δ 9.31 (s, 1H), 9.24 (s, 1H), 8.78 (m, 1H), 8.70 (m, 1H), 8.61 (m, 1H), 8.47 (m, 1H), 7.88 (m, 1H), 7.55 (m, 3H), 2.39 (s, 3H).

Reactant 3 is obtained by the following procedures. A mixture of 3-acetylpyridine (2.47 mol) and N,N-dimethylformamide dimethylacetal (240 mL) is heated at reflux for 16 h. The solvent is removed in vacuo and hexanes (100 mL) added to the residue to crystallize a solid. The solid is recrystallized from dichloromethane-hexanes to give 3-dimethylamino-1-(3-pyridyl)-2-propen-1-one. ¹H NMR (400 MHz, d-chloroform) δ 9.08 (d, J=2.4 Hz, 1H), 8.66 (m, 1H), 8.20 (m, 1H), 7.87 (m, 1H), 7.37 (m, 1H), 5.68 (d, J=16.4 Hz, 1H), 3.18 (s, 3H), 2.97 (s, 3H).

A reactor is charged with concentrated hydrochloric acid (17 mL) followed by stannous chloride dehydrate (0.03 mol). The mixture is stirred for 10 min and then cooled to 0-5° C. A solution of compound 4 (5.6 mmol) in ethyl acetate (3 mL) is slowly added (during 3-4 minutes) while maintaining the temperature at 0-5° C. The reaction mixture is brought to rt and stirred for 1.5 h. To this is added water (50 mL) followed by a slow addition of 50% sodium hydroxide solution (40 mL). The resulting mixture is extracted with chloroform (2×25 mL). The organic layer is washed with water thoroughly and evaporated. The residue is dissolved in ethyl acetate (2 mL), cooled to 0-10° C. and maintained at this temperature for 1 h. The resulting precipitate is collected by filtration and washed with ethyl acetate (1 mL) to provide 1.0 g of 5. ¹H NMR (400 MHz, d-chloroform) δ 9.26 (d, J=2.0 Hz, 1H), 8.71 (m, 1H), 8.48 (d, J=6.8 Hz, 1H), 8.34 (m, 1H), 7.59 (d, J=4.0 Hz, 1H), 7.41 (m, 1H), 7.12 (m, 1H), 7.04 (m, 1H), 6.42 (m, 1H), 3.50 (bs, 2H), 2.24 (s, 3H).

### Synthesis of 3-(4-(pyridin-3-yl)pyrimidin-2-ylamino)-4-methylbenzoic Acid 9

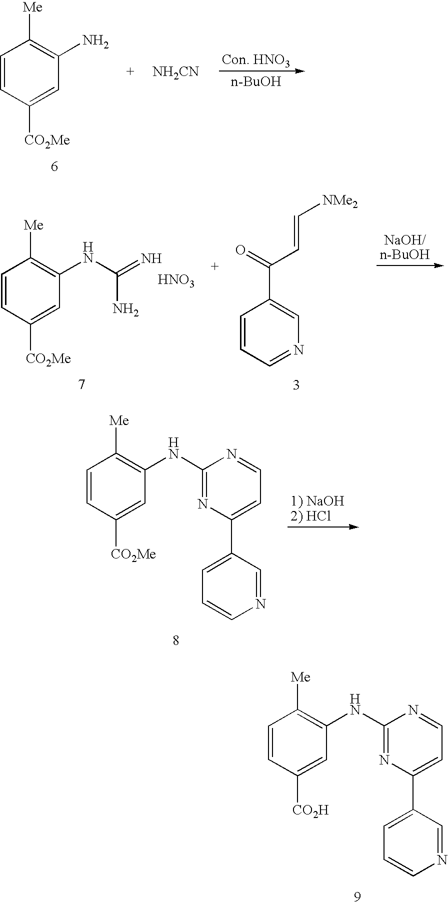

To a solution of 3-amino-4-methyl-benzoic acid methyl ester (0.6 mol) in nBuOH (50 mL) is added 70% nitric acid (2.7 mL) to form the nitrate salt followed by condensation with aqueous cyanamide solution (50% wt., 7 mL, 0.09 mol). The resulting mixture is heated at reflux for 16 h and cooled to rt followed by addition of diethyl ether (100 mL). After cooling at 0° C. for 30 min, filtration and washing with methanol/diethyl:ether (1:1 v/v, 120 mL) afford 3-guanidino-4-methyl-benzoic acid methyl ester nitrate 7.

To 3-guanidino-4-methyl-benzoic acid methyl ester nitrate 7 (0.02 mol) in nBuOH (40 mL) is added 3 (0.02 mol) and sodium hydroxide flakes (0.02 mol). The resulting mixture is heated at reflux for 12 h to yield 8. 1 N aq. NaOH (20 mL) is added to the nBuOH solution of 8 and heated at reflux for 30 min. After cooling to rt 1 N aq. HCl (20 mL) is slowly added to the mixture with vigorous stirring. The product is collected by filtration and washed with water to afford 9. ¹H NMR (400 MHz, d₆-DMSO) δ 9.28 (d, J=1.8 Hz, 1H), 9.08 (s, 1H), 8.7 (dd, J=4.7, 1.5 Hz, 1H), 8.55 (d, J=5.1 Hz, 1H), 8.46 (dt, J=8.0, 1.8 Hz, 1H), 8.31 (s, 1H), 7.65 (dd, J=7.8, 1.5 Hz, 1H), 7.54 (dd, J=7.7, 4.7 Hz, 1H), 7.49 (d, J=5.2 Hz, 1H), 7.37 (d, J=7.9 Hz, 1H), 3.08 (s, 3H). MS (m/z) (M+1)⁺: 307.2.

The same protocol is used to make compounds of type 9 with substitution on the pyridine ring as for the preparation of 3-(4-(5-methoxypyridin-3-yl)pyrimidin-2-ylamino)-4-methylbenzoic acid 43.

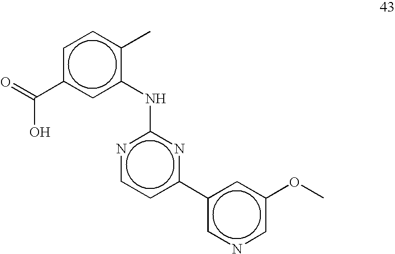

### Synthesis of N1-(4-(5-methoxypyridin-3-yl)pyrimidin-2-yl)-6-methylbenzene-1,3-diamine

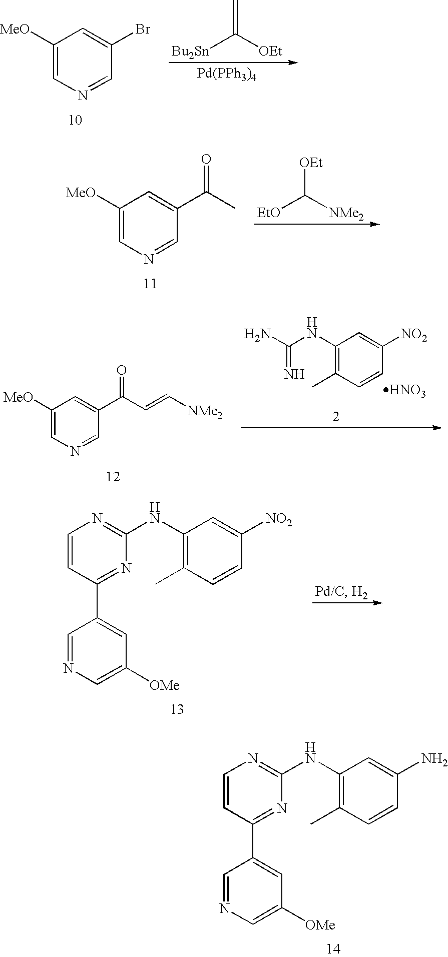

A solution of 3-Bromo-5-methoxypyridine (3 g, 16 mmol), tributyl(1-ethoxyvinyl)stannane (7 mL, 21 mmol) and Pd(PPh₃)₄ (0.92 g, 0.8 mmol) in dry toluene (15 mL) is heated in a microwave at 150° C. for 30 min. After cooling, the mixture is filtered through celite with MeOH and concentrated to give a residue which is purified by silica gel chromatography (ethyl acetate:hexanes=1:1 v/v) to yield 1-(5-methoxypyridin-3-yl)ethanone 11 (1.6 g, 66%). MS (m/z) (M+1)⁺: 152.1.

(E)-3-(dimethylamino)-1-(5-methoxypyridin-3-yl)prop-2-en-1-one 12 is prepared using similar procedures to the synthesis of 3. N-(2-methyl-5-nitrophenyl)-4-(5-methoxypyridin-3-yl)pyrimidin-2-amine 13 is prepared using similar procedures to the synthesis of 4.

To a solution of N-(2-methyl-5-nitrophenyl)-4-(5-methoxypyridin-3-yl)pyrimidin-2-amine 13 (5.0 mmol) in MeOH (20 mL) is added Pd (5% on carbon, 50% wet, 10% weight). The suspension is stirred under hydrogen for 2 h. The reaction is filtered through celite and the celite cake is washed with MeOH. The solvent is removed under reduced pressure to afford 14 which is used without further purification. MS (m/z) (M+1)⁺: 308.2.

Aniline 14 can be used to make the same variety of compounds that are made with aniline 5.

### Synthesis of N-(3-(4-(pyridin-3-yl)pyrimidin-2-ylamino)-4-methylphenyl)-2-chloropyridine-4 carboxamide A-1

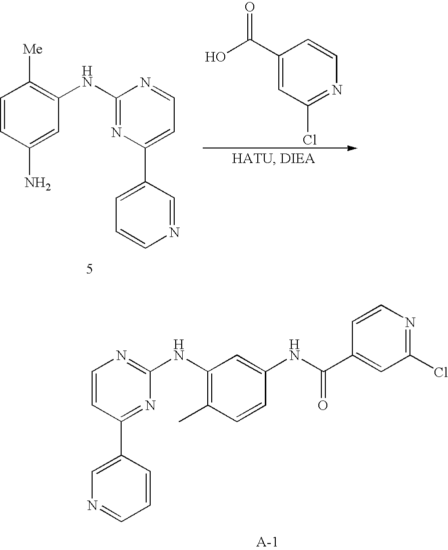

6-Methyl-N1-(4-(pyridin-3-yl)pyrimidin-2-yl)benzene-1,3-diamine 5 (5 mmol), 2-chloro-isonicotinic acid (6 mmol) and HATU (6 mmol) are dissolved in dry DMF (5 mL) at rt. Diisopropylethylamine (6 mmol) is added dropwise to the solution. After 30 min, the mixture is added slowly to saturated aq. NaHCO₃. The solid is filtered, washed with water and dried under vacuum overnight to afford the product A1 as a light yellow solid. ¹H NMR (400 MHz, d₄-methanol) δ 9.3 (s, 1H), 8.65 (m, 1H), 8.6 (m, 1H), 8.55 (d, J=5.1 Hz, 1H), 8.48 (d, J=5.2 Hz, 1H), 8.28 (s, 1H), 7.95 (s, 1H), 7.84 (d, J=5.1 Hz, 1H), 7.56 (m, 1H), 7.4 (dd, J=8.2, 2.1 Hz, 1H), 7.37 (d, J=5.2 Hz, 1H), 7.28 (d, J=8.2 Hz, 1H), 2.33 (s, 3H). MS (m/z) (M+1)⁺: 417.1.

A similar procedure can be used in the preparation of intermediates, 6-chloro-N-(4-methyl-3-(4-(pyridin-3-yl)pyrimidin-2-ylamino)phenyl)nicotinamide 15, 5-formyl-N-(4-methyl-3-(4-(pyridin-3-yl)pyrimidin-2-ylamino)phenyl)furan-2-carboxamide 16 and 5-bromo-N-(4-methyl-3-(4-(pyridin-3-yl)pyrimidin-2-ylamino)phenyl)nicotinamide 17.

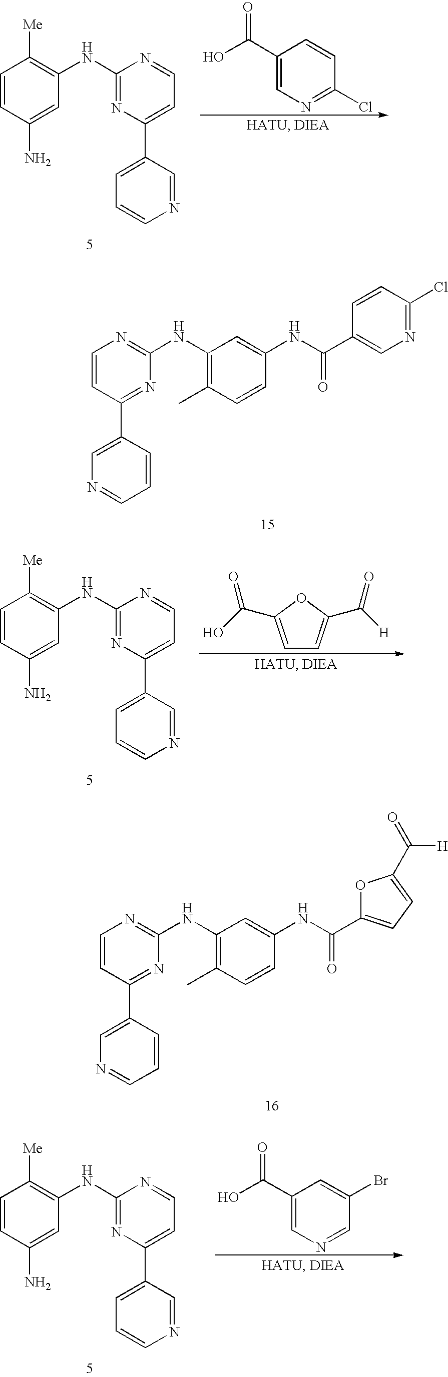

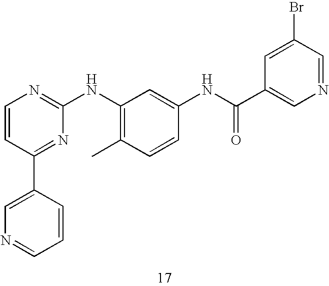

### Synthesis of N-(3-(4-chloropyrimidin-2-ylamino)-4-methylphenyl)-1H-indazole-3-carboxamide 22

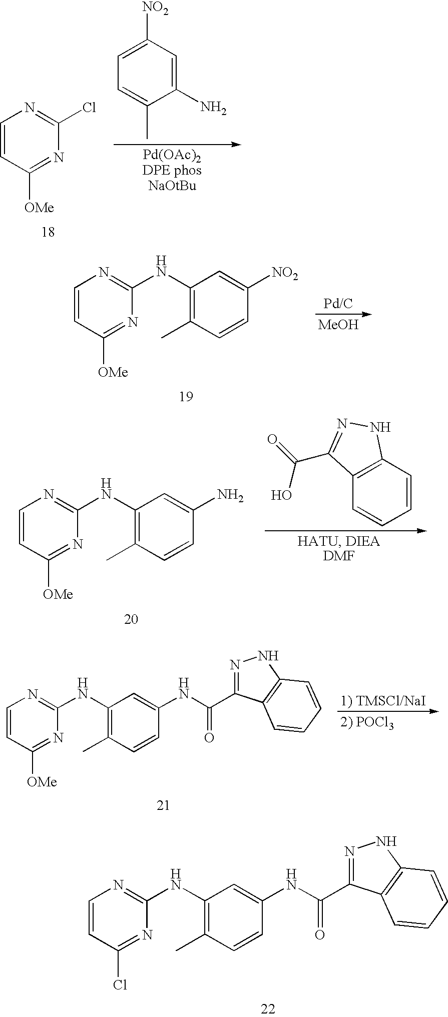

To the mixture of 2-chloro-4-methoxypyrimidine 18 (10.0 mmol), 2-methyl-5-nitrobenzenamine (15.0 mmol), Pd(OAc)₂ (1 mmol), DPE-Phos (1.5 mmol) and NaO-tBu (20.0 mmol) under nitrogen is added 1,4-dioxane (15 mL). The resulting mixture is heated at 150° C. for 20 min under microwave conditions. The reaction mixture is filtered through a pad of celite and the filtrate is diluted in ethyl acetate (100 ml) and washed with water, dried over NaSO₄ and concentrated. The crude product is purified by silica gel column chromatography (ethyl acetate:hexanes=1:4 v/v) to afford 4-methoxy-N-(2-methyl-5-nitrophenyl)pyrimidin-2-amine 19 as a light yellow solid. MS (m/z) (M+1)⁺: 261.1.

To a solution of 4-methoxy-N-(2-methyl-5-nitrophenyl)pyrimidin-2-amine 19 (5.0 mmol) in MeOH (20 mL) is added Pd (5% on carbon, 50% wet, 10% weight). The suspension is stirred under hydrogen for 2 h. The reaction is filtered through celite and the celite cake is washed with MeOH. The solvent is removed under reduced pressure to afford the crude product 20 which is further purified by silica gel column chromatography (ethyl acetate:hexanes=1:2 v/v). MS (m/z) (M+1)⁺: 231.1.

N1-(4-methoxypyrimidin-2-yl)-6-methylbenzene-1,3-diamine 20 (0.65 mmol), 1H-indazole-3-carboxylic acid (0.68 mmol) and HATU (0.79 mmol) are dissolved in dry DMF (4.0 mL) at rt. Diisopropylethylamine (4 mmol) is added to the solution. After 1 h, the mixture is diluted with water (100 mL). The precipitate is filtered, washed with water and dried under vacuum to afford 21 as a light yellow solid. MS (m/z) (M+1)⁺: 375.1.

A mixture of N-(3-(4-methoxypyrimidin-2-ylamino)-4-methylphenyl)-1H-indazole-3-carboxamide 21 (0.53 mmol), TMSCl (2 M in THF, 2.12 mmol) and NaI (2.12 mmol) in ACN (2 mL) is heated at 140° C. for 20 min under microwave condition. To the reaction mixture is added aqueous 2M Na₂CO₃ (50 mL) and then extracted with ethyl acetate (100 mL×2). The organic layer is washed with water, dried on Na₂SO₄ and concentrated to afford a residue. To this residue is added POCl₃ (5 ml) and the resulting mixture is refluxed for 15 min. Excess POCl₃ is removed in vacuo. The residue is dissolved in ethyl acetate (100 mL), washed with Na₂CO₃ solution, dried over Na₂SO₄ and filtered. The solvent is evaporated in vacuo to afford the crude product 22 which is purified by silica gel column chromatography (ethyl acetate:hexanes=1:2 v/v). ¹H NMR (400 MHz, d-chloroform) δ 8.9 (s, 1H), 8.43 (d, J=8.2 Hz, 1H), 8.22-8.29 (m, 3H), 7.43-7.58 (m, 3H), 7.33 (t, J=7.2 Hz, 1H), 7.21 (d, J=8.4 Hz, 1H), 2.31 (s, 3H). MS (m/z) (M+1)⁺: 379.1.

Compounds similar to 22 can be made by coupling compound 20 with different carboxylic acids as in the preparation of 25.

### Synthesis of N-(3-(4-chloropyrimidin-2-ylamino)-4-methylphenyl)-1-ethyl-3-methyl-1H-pyrazole-5-carboxamide 25

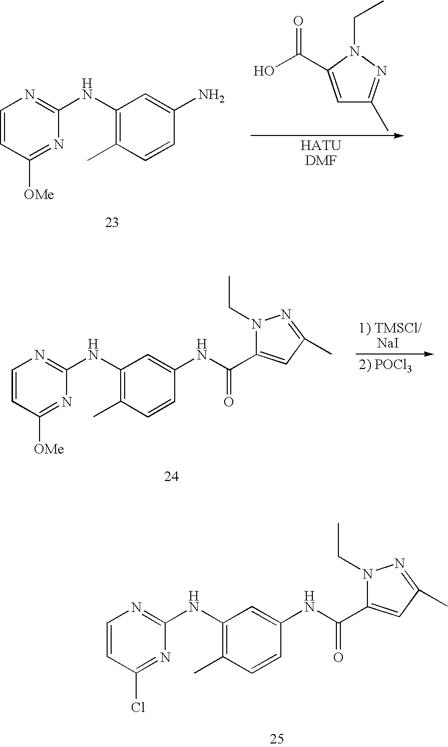

N1-(4-methoxypyrimidin-2-yl)-6-methylbenzene-1,3-diamine 23 (0.65 mmol), 1-ethyl-3-methyl-1H-pyrazole-5-carboxylic acid (0.68 mmol) and HATU (0.79 mmol) are dissolved in dry DMF (4.0 mL) at rt. Diisopropylethylamine (4 mmol) is added to the solution. After 1 h, the mixture is diluted with water (100 mL). The precipitate is filtered, washed with water and dried under vacuum to afford 24 as a light yellow solid. ¹H NMR (400 MHz, d-chloroform) δ 8.49 (s, 1H), 8.12 (d, J=5.8 Hz, 1H), 7.69 (s, 1H), 7.14-7.20 (m, 2H), 6.94 (bs, 1H), 6.38 (s, 1H), 6.21 (d, J=5.8 Hz, 1H), 4.50-4.56 (m, 2H), 3.98 (s, 3H), 2.31 (s, 3H), 2.29 (s, 3H), 1.43 (t, J=7.2 Hz, 3H). MS (m/z) (M+1)⁺: 367.2.

A mixture of N-(3-(4-methoxypyrimidin-2-ylamino)-4-methylphenyl)-1-ethyl-3-methyl-1H-pyrazole-5-carboxamide 24 (0.53 mmol), TMSCl (2 M in THF, 2.12 mmol) and NaI (2.12 mmol) in ACN (2 mL) is heated at 140° C. for 20 min under microwave condition. To the reaction mixture is added aqueous 2M Na₂CO₃ (50 mL) and is extracted with ethyl acetate (100 mL×2). The organic layer is washed with water, dried over Na₂SO₄ and concentrated to afford a residue. To the residue is added POCl₃ (5 ml) and the resulting mixture is refluxed for 15 min. Excess POCl₃ is removed in vacuo. The residue is dissolved in ethyl acetate (100 mL), washed with Na₂CO₃ solution, dried over Na₂SO₄ and filtered. The solvent is evaporated in vacuo to afford the crude product 25 which is further purified by silica gel column chromatography (ethyl acetate:hexanes=1:2 v/v). MS (m/z) (M+1)⁺: 371.1.

### Synthesis of 1-(4-cyanophenyl)-3-methyl-1H-pyrazole-5-carboxylic Acid 28

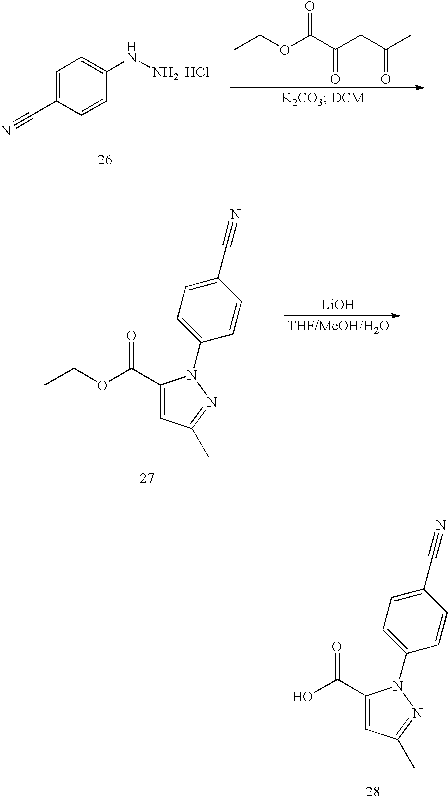

To a solution of 4-hydrazinylbenzonitrile hydrochloride 26 (2.06 mmol) in dichloromethane at 0° C. is added potassium carbonate (1.59 mmol) followed by ethyl 2,4-dioxopentanoate (3.16 mmol). The reaction mixture is left to stir overnight at rt. The reaction mixture is diluted with dichloromethane, washed with water and brine, dried over sodium sulfate and the solvent is removed to afford the crude product 27 which is used without further purification.

Ethyl 1-(4-cyanophenyl)-3-methyl-1H-pyrazole-5-carboxylate 27 is dissolved in a solution of THF/MeOH/H₂O (3:2:1 v/v) and 6 N lithium hydroxide (3 eq) is added. The mixture is stirred overnight. The solvent is removed in vacuo and the residue is diluted in H₂O, extracted with dichloromethane (3 times) and the pH of the aqueous layer is adjusted to pH 5. The precipitate is filtered and dried to yield 1-(4-cyanophenyl)-3-methyl-1H-pyrazole-5-carboxylic acid 28 which is used to make compounds A-71-A-73. MS (m/z) (M+1)⁺: 228.1.

### Synthesis of 6-methyl-N1-(4-(5-morpholinopyridin-3-yl)pyrimidin-2-yl)benzene-1,3-diamine 33

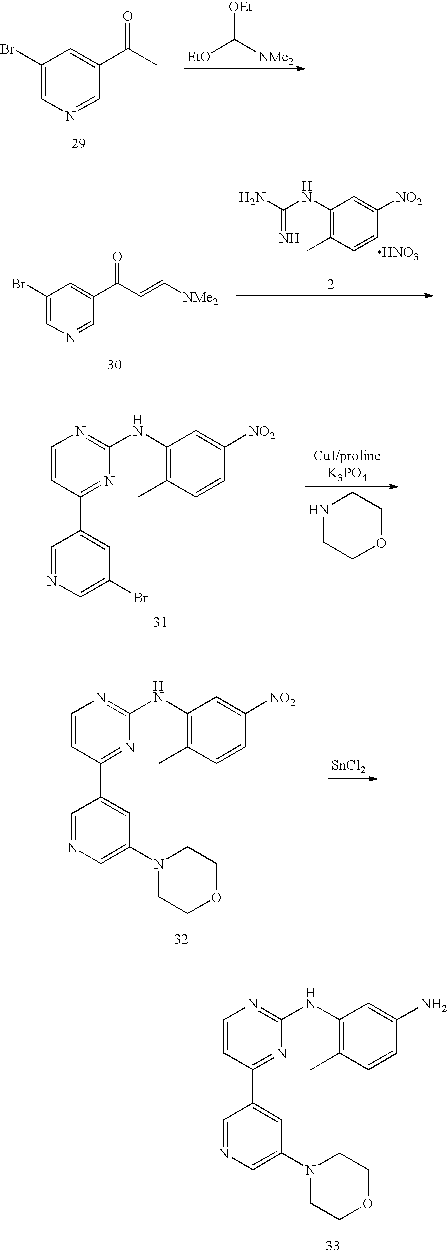

(E)-1-(5-Bromopyridin-3-yl)-3-(dimethylamino)prop-2-en-1-onene 30 is prepared from 29 using similar procedures to the synthesis of 3. 4-(5-Bromopyridin-3-yl)-N-(2-methyl-5-nitrophenyl)pyrimidin-2-amine 31 is prepared from 30 using similar procedures to the synthesis of 4.

Compound 31 (152 mg, 0.4 mmol), morpohline (1.2 mmol), K₃PO₄ (168 mg, 0.8 mmol), CuI (15 mg, 0.04 mmol) and L-proline (19 mg, 0.08 mmol) are heated in dry DMSO under nitrogen at 90° C. for 16 h. The mixture is diluted with EtOAc and washed with water. After removal of the solvent in vacuo, the residue containing mainly 32 is used in the next step without further purification. MS (m/z) (M+1)⁺: 393.2.

Crude N-(2-methyl-5-nitrophenyl)-4-(5-morpholinopyridin-3-yl)pyrimidin-2-amine 32 is heated with SnCl₂ (0.78 g, 4 mmol) in EtOH (5 mL) at reflux for 2 h. Aq. 1 N NaOH is added until pH>14. The mixture is filtered and washed with dichloromethane. The combined organic phases are concentrated and purified by preparative HPLC to give 33. MS (m/z) (M+1)⁺: 363.2.

Compounds similar to 33 can be made by coupling compound 31 with different amines.

### Synthesis of N1-(4-(5-(difluoromethoxy)pyridin-3-ylpyrimidin-2-yl)-6-methylbenzene-1,3-diamine 36

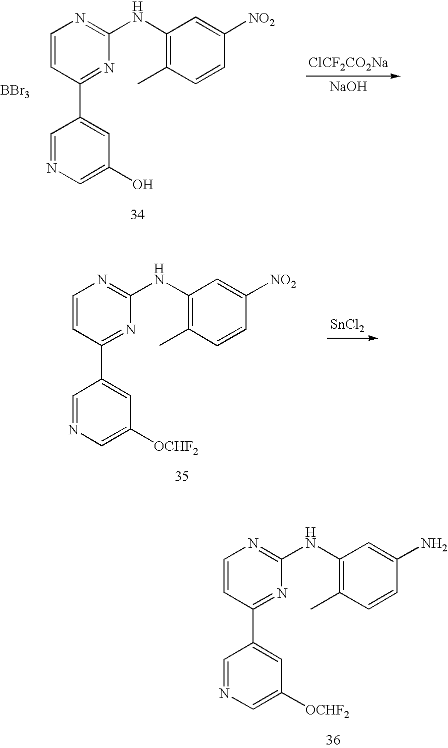

4-(5-Methoxypyridin-3-yl)-N-(2-methyl-5-nitrophenyl)pyrimidin-2-amine 13 (3 g, 10 mmol) is suspended in dry dichloromethane. BBr₃ (3 mL, 32 mmol) is introduced slowly at rt. The mixture is stirred for 3 days and quenched by slow addition to ice water. Solid NaOH is added until pH>14. The mixture is extracted with dichloromethane. Concentrated aqueous HCl is added slowly to the aqueous phase until pH=7. The solid is filtered and dried under vacuum to give 34 which is used without further purification.

5-(2-(2-methyl-5-nitrophenylamino)pyrimidin-4-yl)pyridin-3-ol 34 (97 mg, 0.3 mmol) is heated with NaOH (24 mg, 0.6 mmol) and ClCF₂CO₂Na (92 mg, 0.6 mmol) in dry DMF (1 mL) in a microwave oven at 180° C. for 45 min. The residue is dissolved in ethyl acetate and washed with water. After concentration, the crude mixture is purified by silica gel column chromatography (ethyl acetate:hexanes=1:1 v/v) to give 35. MS (m/z) (M+1)⁺: 374.1.

35 (50 mg, 0.13 mmol) is heated with SnCl₂ (0.39 g, 2 mmol) in EtOH (2 mL) at reflux for 2 h. 1N NaOH is added until pH>14. The mixture is filtered and washed with dichloromethane. The combined organic phases are concentrated to give 36 which is used without further purification. MS (m/z) (M+1)⁺: 344.2.

### Synthesis of N1-(4-(isoquinolin-4-yl)pyrimidin-2-yl)-6-methylbenzene-1,3-diamine 40

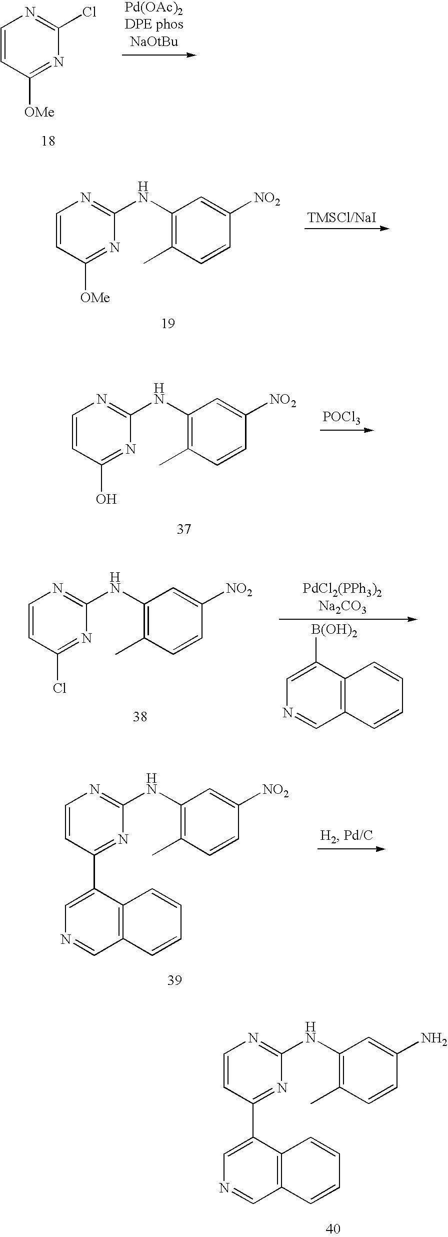

A mixture of 19 (1 g, 3.8 mmol), TMSCl (1M in dichloromethane, 6.7 mL, 6.7 mmol) and NaI (1.45 g, 7.7 mmol) in ACN (10 mL) is heated at 120° C. for 20 min under microwave conditions. To the reaction mixture is added aqueous 2 M Na₂CO₃ (50 mL) and dichloromethane (2×100 mL). The organic layer is separated, washed with water, dried over Na₂SO₄ and concentrated to afford a residue of crude 2-(2-methyl-5-nitrophenylamino)pyrimidin-4-ol 37. To this residue is added POCl₃ (5 mL) and the resulting mixture is refluxed for 2 h. Excess POCl₃ is removed in vacuo. The residue is dissolved in dichloromethane (100 mL), washed with Na₂CO₃ solution, dried over Na₂SO₄ and filtered. The solvent is evaporated in vacuo to afford the crude product 38 which is used without further purification. MS (m/z) (M+1)⁺: 265.2, 267.2.

4-Chloro-N-(2-methyl-5-nitrophenyl)pyrimidin-2-amine 38 (1 g, 4 mmol), isoquinolin-4-ylboronic acid (1 g, 4 mmol) and Pd(PPh₃)₂Cl₂ (140 mg, 0.2 mmol) are added to a 40-mL vial equipped with a stir bar. The vial is vented and refilled with nitrogen five times. 1,4-Dioxane (20 mL) and aqueous 3 M Na₂CO₃ (8 mL, 24 mmol) are added by syringe. The vial is sealed and heated at 150° C. for 10 min under microwave conditions. The mixture is filtered and diluted with dichloromethane. After washing with 1 N NaOH (50 mL), the organic phase is washed with 1N HCl (20 mL). The aqueous phase is stored in freezer overnight to give product 39 as a solid precipitate which is filtered and dried. MS (m/z) (M+1)⁺: 358.2.

4-(Isoquinolin-4-yl)-N-(2-methyl-5-nitrophenyl)pyrimidin-2-amine 39 (200 mg, 0.55 mmol) is dissolved in MeOH (10 mL) and stirred at rt under 1 atm of hydrogen in the presence of 5% Pd/C (140 mg) for 3 h. After filtration, the solvent is removed to yield N-1-(4-(isoquinolin-4-yl)pyrimidin-2-yl)-6-methylbenzene-1,3-diamine 40 which is used without further purification. MS (m/z) (M+1)⁺: 328.2.

### Synthesis of N-(3-(4-(5-bromopyridin-3-yl)pyrimidin-2-ylamino)-4-methylphenyl)-2-methyl-5-(trifluoromethyl)oxazole-4-carboxamide 42

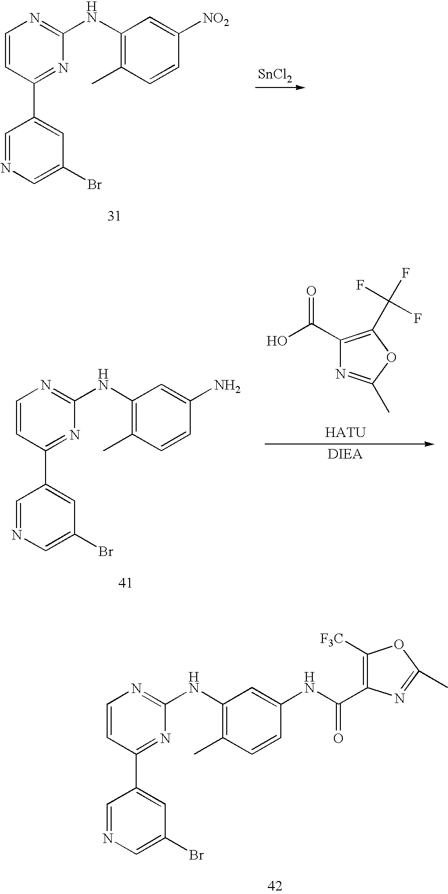

4-(5-Bromopyridin-3-yl)-N-(2-methyl-5-nitrophenyl)pyrimidin-2-amine 31 (210 mg, 0.55 mmol) is heated with SnCl₂ (311 mg, 1.64 mmol) in EtOH (5 mL) at reflux for 2 h. Aq. 1 N NaOH is added until pH>14. The mixture is filtered and washed with dichloromethane. The combined organic phases are concentrated to give 41 which is used without further purification. MS (m/z) (M+1)⁺: 356.2, 358.2.

Crude N-1-(4-(5-bromopyridin-3-yl)pyrimidin-2-yl)-6-methylbenzene-1,3-diamine 41 (0.5 mmol) is stirred with 2-methyl-5-(trifluoromethyl)oxazole-4-carboxylic acid (107 mg, 0.55 mol), HATU (251 mg, 0.66 mmol) and DIPEA (0.35 mL, 2 mmol) in dry DMF (2 mL) at rt for 30 min. The mixture is purified by preparative HPLC to yield N-(3-(4-(5-bromopyridin-3-yl)pyrimidin-2-ylamino)-4-methylphenyl)-2-methyl-5-(trifluoromethyl)oxazole-4-carboxamide 42. MS (m/z) (M+1)⁺: 533.3, 535.3.

### Example 1

### N-(3-(4-(pyridin-3-yl)pyrimidin-2-ylamino)-4-methylphenyl)-5-chloro-1H-indole-2-carboxamide A-6

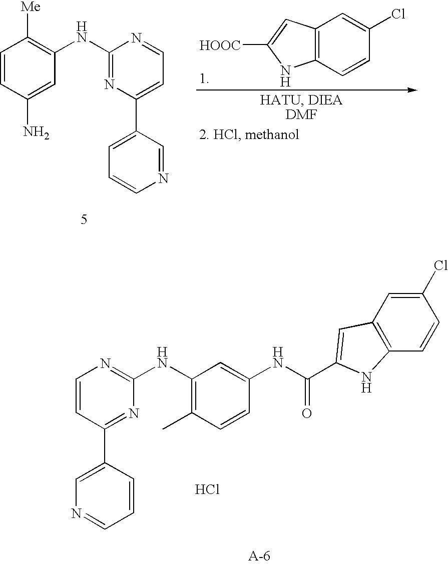

6-Methyl-N1-(4-(pyridin-3-yl)pyrimidin-2-yl)benzene-1,3-diamine 5 (0.27 mmol), 5-chloroindole-2-carboxylic acid (0.30 mmol) and HATU (0.32 mmol) are dissolved in dry DMF (1.5 mL) at rt. Diisopropylethylamine (6 mmol) is added to the solution. After 12 h, the mixture is diluted with methanol (5 mL). The precipitate is filtered, washed with methanol and dried under vacuum to afford a light yellow solid, which is then suspended in methanol and treated with HCl (0.2 mL of a 2.0M solution in 1,4-dioxane). After 1 h the mixture is reduced to dryness and dried under vacuum to afford the product A6 as a bright orange solid. ¹H NMR (400 MHz, d₆-DMSO) δ 11.96 (s, 1H), 10.30 (s, 1H), 9.43 (bs, 1H), 9.14 (s, 1H), 8.85 (m, 2H), 8.60 (d, J=4.8 Hz, 1H), 8.16 (bs, 1H), 7.85 (bs, 1H), 7.77 (d, J=2.0 Hz, 1H), 7.52 (m, 2H), 7.48 (d, J=8.5 Hz, 1H) 7.43 (bs, 1H), 7.25 (d, J=6.0 Hz, 1H), 7.23 (dd, J=8.5, 2.0 Hz, 1H), 2.25 (s, 3H). MS (m/z) (M+1)⁺: 455.1.

Anilines 14, 33, 36, 40 or others made in similar fashion are used to make other type A final compounds using a similar procedure to make A-6 from intermediate 5.

### Example 2

### N-(3-(4-(Pyridin-3-yl)pyrimidin-2-ylamino)-4-methylphenyl)-2-morpholinopyridine-4-carboxamide B-1

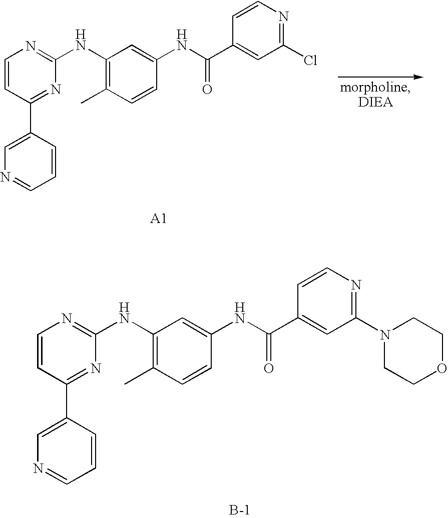

N-(3-(4-(pyridin-3-yl)pyrimidin-2-ylamino)-4-methylphenyl)-2-chloropyridine-4 carboxamide A-1 (2 mmol), morpholine (10 mmol) and diisopropylethylamine (4 mmol) are heated at 250° C. in a microwave oven for 8 min. The mixture is purified by preparative HPLC (ACN/water gradient 10-70%). The combined solution of product is concentrated and solid Na₂CO₃ is added until pH=10. Extraction with dichloromethane and drying over anhydrous K₂CO₃ affords a mixture of solid and oil after concentration which is further triturated in MeOH/Et₂O. After filtration, the product B1 is obtained as off-white solid. ¹H NMR (400 MHz, d₆-acetone) δ 9.47 (s, 1H), 9.22 (s, 1H), 8.56 (dd, J=4.7, 1.6 Hz, 1H), 8.45 (m, 1H), 8.41 (m, 1H), 8.4 (m, 1H), 8.15 (d, J=5.1 Hz, 1H), 7.87 (s, 1H), 7.37 (m, 2H), 7.3 (d, J=5.1 Hz, 1H), 7.17 (s, 1H), 7; 11 (d, J=8.2 Hz, 1H), 7.03 (dd, J=5.1, 1.2 Hz, 1H), 3.63 (t, J=4.7 Hz, 4H), 3.44 (t, J=4.7 Hz, 1H), 2.24 (s, 3H). MS (m/z) (M+1)⁺: 468.1.

A similar procedure utilizing 6-chloro-N-(4-methyl-3-(4-(pyridin-3-yl)pyrimidin-2-ylamino)phenyl)nicotinamide 15 as intermediate was used to prepare examples B-12 and B-13, B-16 and B-17.

### Example 3

### 2-(3-Hydroxypropoxy)-N-(3-(4-(pryridin-3-yl)pyrimidin-2-ylamino)-4-methylphenyl)pyridine-4-carboxamide C-2

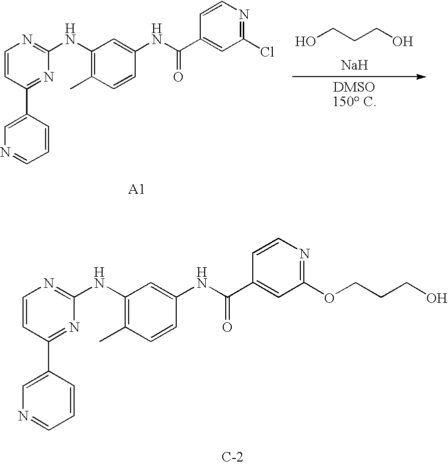

N-(3-(4-(pyridin-3-yl)pyrimidin-2-ylamino)-4-methylphenyl)-2-chloropyridine-4 carboxamide A1 (0.048 mmol) is added to a mixture of propane-1,3-diol (0.48 mmol) and NaH (0.24 mmol) in DMSO (1 mL) and the reaction mixture is heated at 150° C. for 2 h. The mixture is purified by preparative HPLC (ACN/water gradient 10-70%) to afford the corresponding product C2 as a TFA salt. ¹H NMR (400 MHz, d₆-DMSO) δ 10.40 (s, 1H), 9.41 (d, J=1.44 Hz, 1H), 9.14 (s, 11H), 8.83-8.88 (m, 2H), 8.60 (d, J=5.2 Hz, 1H), 8.15 (s, 1H), 7.83-7.88 (m, 1H), 7.53 (d, J=5.2 Hz, 1H), 7.41-7.49 (m, 2H), 7.32 (s, 1H), 7.23 (d, J=8.3 Hz, 1H), 4.38 (t, J=6.5 Hz, 2H), 3.57 (t, J=6.2 Hz, 2H), 2.24 (s, 3H), 1.85-1.93 (m, 2H). MS (m/z) (M+1)⁺: 457.1.

A similar procedure utilizing 6-chloro-N-(4-methyl-3-(4-(pyridin-3-yl)pyrimidin-2-ylamino)phenyl)nicotinamide 15 as intermediate was used to prepare examples C-9 to C-12.

### Example 4

### 3-(4-(Pyridin-3-yl)pyrimidin-2-ylamino)-N-(3,4-dihydro-3-oxo-2H-benzo[b][1,4]oxazin-6-yl)-4-methylbenzamide D-2

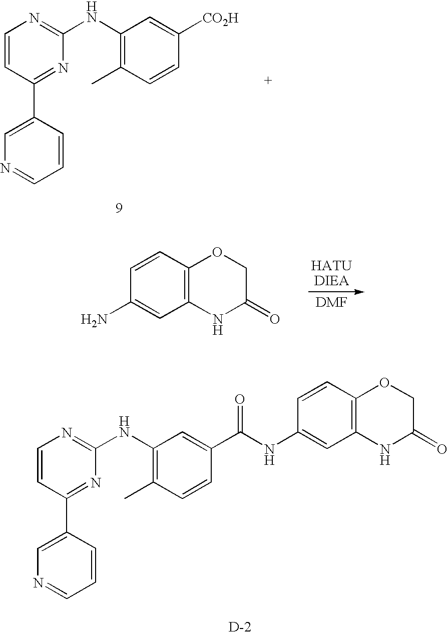

3-(4-(pyridin-3-yl)pyrimidin-2-ylamino)-4-methylbenzoic acid 9 (0.1 mmol), 6-amino-2H-benzo[b][1,4]oxazin-3(4H)-one (0.1 mmol) and HATU (0.15 mmol) are dissolved in dry DMF (0.5 mL) at rt. Diisopropylethylamine (0.50 mmol) is added to the solution. The reaction mixture is stirred for 1 h at rt. HPLC purification affords the target compound D2 as a TFA salt. ¹H NMR (400 MHz, d₆-DMSO) δ 10.78 (s, 1H), 10.15 (s, 1H), 9.29 (d, J=1.7 Hz, 1H), 9.18 (s, 1H), 8.74 (dd, J=1.4, 4.9 Hz, 1H), 8.52-8.58 (m, 2H), 8.23 (d, J=1.3 Hz, 1H), 7.71 (dd, J=1.7, 7.9 Hz, 1H), 7.59-7.64 (m, 1H), 7.54 (d, J=2.4 Hz, 1H), 7.49 (d, J=5.2 Hz, 1H), 7.40 (d, J=8.1 Hz, 1H), 7.23 (dd, J=2.4, 8.7 Hz, 1H), 6.92 (d, J=8.7 Hz, 1H), 4.54 (s, 2H), 2.34 (s, 3H). MS (m/z) (M+1)⁺: 453.2.

A similar procedure utilizing 3-(4-(5-methoxypyridin-3-yl)pyrimidin-2-ylamino)-4-methylbenzoic acid 43 as intermediate was used to prepare examples D-5 to D-12.

### Example 5

### N-(3-(4-(5-Methoxypyridin-3-yl)pyrimidin-2-ylamino)-4-methylphenyl)-1-ethyl-3-methyl-1H-pyrazole-5-carboxamide E-4

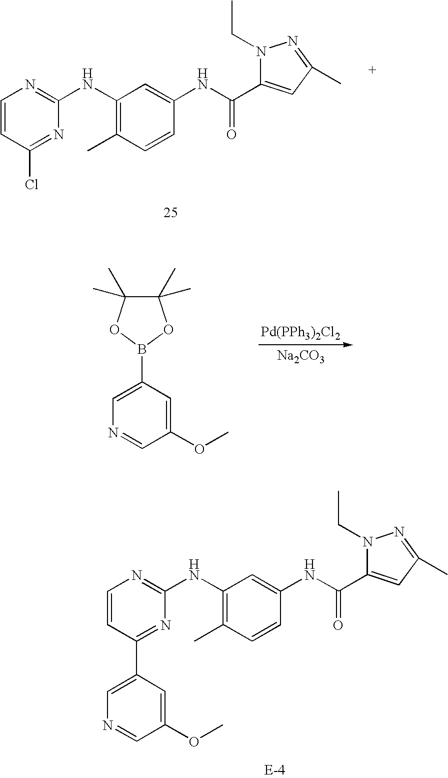

N-(3-(4-chloropyrimidin-2-ylamino)-4-methylphenyl)-1-ethyl-3-methyl-1H-pyrazole-5-carboxamide 25 (0.021 mmol), 3-methoxy-5-(4,4,5,5-tetramethyl-1,3,2-dioxaborolan-2-yl)pyridine (0.025 mmol) and Pd(PPh₃)₂Cl₂ (0.0014 mmol) are added to a 10-mL Schlenk flask equipped with a stir bar. The flask is evacuated and refilled with nitrogen five times. 1,4-Dioxane (0.8 mL) and aqueous Na₂CO₃ (3.1 M, 0.12 mmol) are added by syringe. The Schlenk flask is sealed and heated at 150° C. for 10 min under microwave conditions. HPLC purification gives product E4 as a TFA salt. ¹H NMR (400 MHz, d₄-methanol) δ 9.12 (s, 1H), 8.64 (s, 1H), 8.59-8.62 (m, 1H), 8.55 (d, J=5.4 Hz, 1H), 8.23 (s, 1H), 7.54 (d, J=5.4 Hz, 1H), 7.27-7.35 (m, 2H), 6.70 (s, 1H), 4.45-4.52 (m, 2H), 4.03 (s, 3H), 2.33 (s, 3H), 2.28 (s, 3H), 1.36 (t, J=7.1 Hz, 3H). MS (m/z) (M+1)⁺: 444.2.

A similar procedure utilizing N-(3-(4-chloropyrimidin-2-ylamino)-4-methylphenyl)-1H-indazole-3-carboxamide 22 as intermediate was used to prepare examples E-1 to E-3.

### Example 6

### 5-((Diethylamino)methyl)-N-(4-methyl-3-(4-(pyridin-3-yl)pyrimidin-2-ylamino)phenyl)furan-2-carboxamide F-1

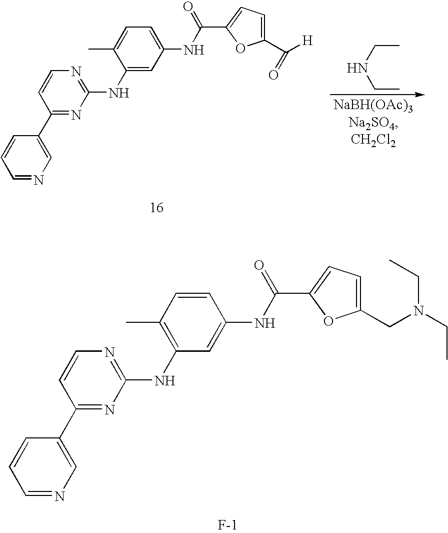

A mixture of N-(3-(4-(pyridin-3-yl)pyrimidin-2-ylamino)-4-methylphenyl)-4-formylcyclopenta-1,3-dienecarboxamide 16 (0.03 mmol), diethylamine (0.09 mmol) and excess Na₂SO₄ in dichloromethane (0.5 mL) is stirred at rt for 1 h. Then NaBH(OAc)₃ (0.15 mmol) is added and stirred overnight. The mixture is purified by preparative HPLC (ACN/water gradient 10-70%) to afford the corresponding product F1 as a TFA salt. ¹H NMR (400 MHz, d₆-DMSO) δ 10.14 (s, 1H), 9.28 (s, 1H), 9.01 (s, 1H), 8.70 (d, J=3.6 Hz, 1H), 8.52 (m, 2H), 7.99 (s, 1H), 7.57 (m, 1H), 7.44 (m, 1H), 7.23 (m, 1H), 6.94 (m, 1H), 4.50 (s, 2H), 3.13 (m, 4H), 2.55 (s, 3H), 1.25 (t, J=7.2 Hz, 6H). MS (m/z) (M+1)⁺: 479.2.

### Example 7

### N-(4-methyl-3-(4-(pyridin-3-yl)pyrimidin-2-ylamino)phenyl)-5-morpholinonicotinamide G-1

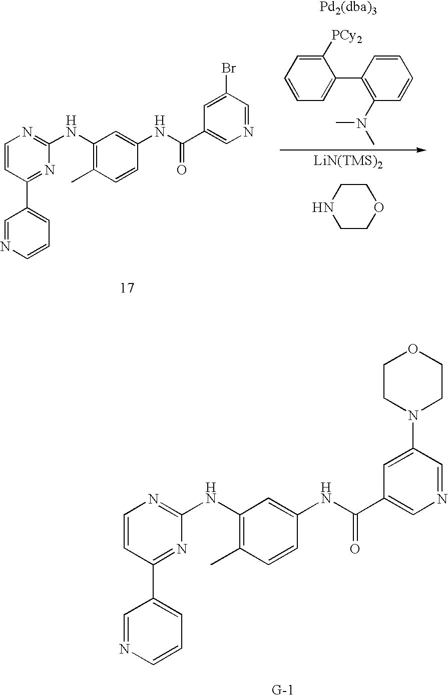

An oven-dried vial is charged with Pd₂(dba)₃ (0.011 mmol), 2′-(dicyclohexylphosphino)-N,N-dimethylbiphenyl-2-amine (0.013 mmol) and N-(3-(4-(pyridin-3-yl)pyrimidin-2-ylamino)-4-methylphenyl)-5-bromopyridine-3-carboxamide (0.216 mmol). The vial is evacuated and back-filled with N₂. Then LiN(TMS)₂ solution (1M in THF, 1.0 mL), 1,4-dioxane (1 mL) and morpholine (0.26 mmol) are added via syringe. The mixture is heated at 140° C. under microwave conditions for 45 min. The resulting mixture is purified by preparative HPLC (ACN/water gradient 10-70%) to afford the corresponding product G1 as a TFA salt. ¹H NMR (400 MHz, d₆-DMSO) δ 10.5 (s, 1H), 9.35 (s, 1H), 9.07 (s, 1H), 8.76 (d, J=4.0 Hz, 1H), 8.62 (m, 3H), 8.10 (m, 1H), 8.02 (s, 1H), 7.67 (m, 1H), 7.48 (m, 1H), 7.23 (m, 1H), 3.79 (t, J=4.8 Hz, 4H), 3.35 (t, J=4.8 Hz, 4H), 2.25 (s, 3H). MS (m/z) (M+1)⁺: 468.2.

### Example 8

### 1-(3-(4-(5-methoxyridin-3-yl)pyrimidin-2-ylamino)-4-methylphenyl)-3-(pyridin-2-yl)urea H-6

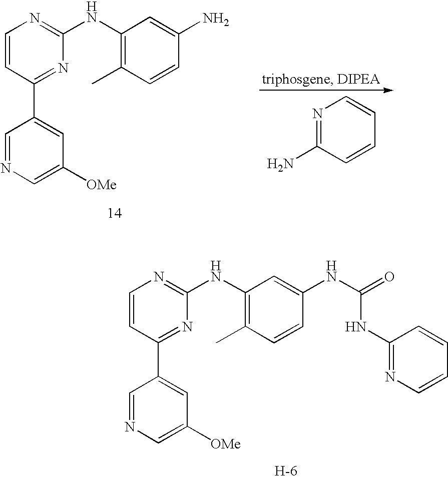

Pyridin-2-amine (5 mg, 0.05 mmol) is mixed with triphosgene (4.9 mg, 0.017 mmol) in dry THF at rt for 20 min. N-1-(4-(5-methoxypyridin-3-yl)pyrimidin-2-yl)-6-methylbenzene-1,3-diamine 14 (15 mg, 0.05 mmol) is added and reaction is quenched by adding MeOH after 20 min. Solvent is removed and the residue is purified by preparative HPLC to give urea H-6. ¹H NMR (400 MHz, d₆-DMSO) δ 10.46 (s, 1H), 8.98 (s, 1H), 8.91 (s, 1H), 8.54 (d, J=5 Hz, 1H), 8.44 (d, J=2.4 Hz, 1H), 8.05 (s, 1H), 7.9 (s, 1H), 7.78 (t, J=6.8 Hz, 1H), 7.48 (m, 2H), 7.2 (m, 2H), 7.03 (1H, J=5.7 Hz, 1H), 3.86 (s, 3H), 2.55 (s, 1H), 2.21 (s, 3H). MS (m/z) (M+1)⁺: 434.2.

Anilines 14, 33, 36, 40 or others made in similar fashion are used to make other type H final compounds using a similar procedure to make H-6 from intermediate 14.

### Example 9

### N-(3-(4-(5-((2S,6R)-2,6-dimethylmopholino)pyridin-3-yl)pyrimidin-2-ylamino)-4-methylphenyl)-2-methyl-5-(trifluoromethyl)oxazole-4-carboxamide I-1

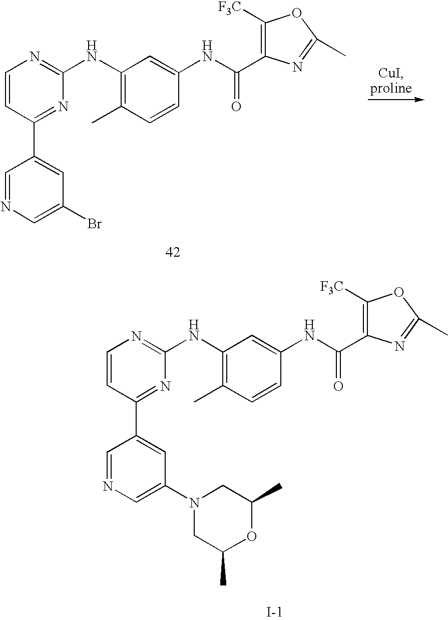

Compound 42 (30 mg, 0.056 mmol), dimethylmorpholine (13 mg, 0.12 mmol), K₃PO₄ (24 mg, 0.11 mmol), CuI (2.2 mg, 0.006 mmol) and L-proline (2.7 mg, 0.012 mmol) are heated in dry DMSO under nitrogen at 90° C. for 16 h. The mixture is filtered and purified by preparative HPLC to give I-1. ¹H NMR (400 MHz, d₆-DMSO) δ 10.52 (s, 1H), 9.04 (s, 1H), 8.7 (dd, J=5.5, 3.4 Hz, 1H), 8.5 (m, 2H), 8.06 (s, 1H), 8.02 (s, 1H), 7.53 (d, J=6.8 Hz, 1H), 7.48 (d, J=3.6 Hz, 1H), 7.21 (d, J=8.3 Hz, 1H), 3.68 (m, 2H), 2.6 (s, 3H), 2.34 (m, 4H), 2.21 (s, 3H), 1.13 (d, J=5.3 Hz, 6H). MS (m/z) (M+1)⁺: 568.3.

By repeating the procedures described in the above examples, using appropriate starting materials, the following compounds of Formula I, as identified in Table 1, are obtained.

TABLE 1

| Example | | MS | |
|---|---|---|---|
| # | Structure | [M + 1]⁺ | NMR |
| A-1 | 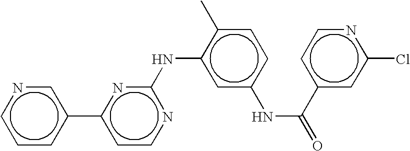 | 417.1 | ¹H NMR (400 MHz, d₄-methanol) δ 9.3 (s, 1H), 8.65 (m, 1H), 8.6 (m, 1H), 8.55 (d, J = 5.1 Hz, 1H), 8.48 (d, J = 5.2 Hz, 1H), 8.28 (s, 1H), 7.95 (s, 1H), 7.84 (d, J = 5.1 Hz, 1H), 7.56 (m, 1H), 7.4 (dd, J = 8.2, 2.1 Hz, 1H), 7.37 (d, J = 5.2 Hz, 1H), 7.28 (d, J = 8.2 Hz, 1H), 2.33 (s, 3H). |
| A-2 | 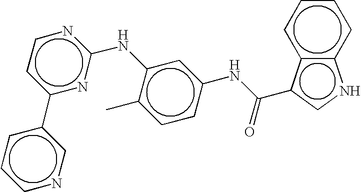 | 421.1 | |
| A-3 | 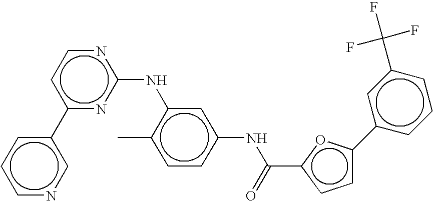 | 516.1 | ¹H NMR (400 MHz, d₆-DMSO) δ 11.7 (s, 1H), 10.2 (s, 1H), 9.29 (s, 1H), 9.01 (s, 1H), 8.69 (d, J = 3.2 Hz, 1H), 8.52 (m, 2H), 8.09 (s, 1H), 7.67 (d, J = 8.4 Hz, 1H), 7.56 (m, 2H), 7.25 (m, 3H), 7.08 (d, J = 7.2 Hz, 2H), 2.24 (s, 3H). |
| A-4 | 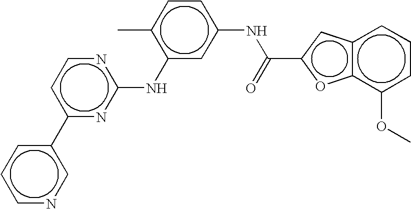 | 452.2 | ¹H NMR (400 MHz, d₆-DMSO) δ 10.4 (s, 1H), 9.33 (s, 1H), 9.06 (s, 1H), 8.76 (d, J = 4.4 Hz, 1H), 8.63 (d, J = 8.0 Hz, 1H), 8.55 (d, J = 4.8 Hz, 1H), 8.12 (s, 1H), 7.76 (m, 1H), 7.67 (m, 1H), 7.49 (m, 1H), 7.36 (d, J = 8.0 Hz, 1H), 7.28 (m, 1H), 7.10 (d, J = 8.0 Hz, 1H), 2.24 (s, 3H). |
| A-5 | 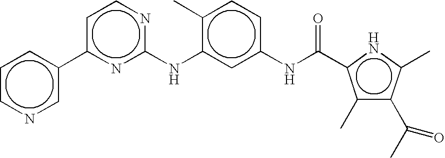 | 441.2 | |
| A-6 | 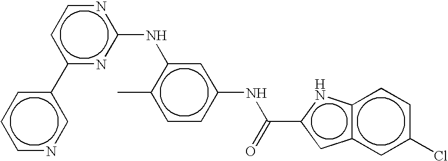 | 455.2 | ¹H NMR (400 MHz, d₆-DMSO) δ 11.96 (s, 1H), 10.30 (s, 1H), 9.43 (bs, 1H), 9.14 (s, 1H), 8.85 (m, 2H), 8.60 (d, J = 4.8 Hz, 1H), 8.16 (bs, 1H), 7.85 (bs, 1H), 7.77 (d, J = 2.0 Hz, 1H), 7.52 (m, 2H), 7.48 (d, J = 8.5 Hz, 1H) 7.43 (bs, 1H), 7.25 (d, J = 6.0 Hz, 1H), 7.23 (dd, J = 8.5, 2.0 Hz, 1H), 2.25 (s, 3H). |
| A-7 | 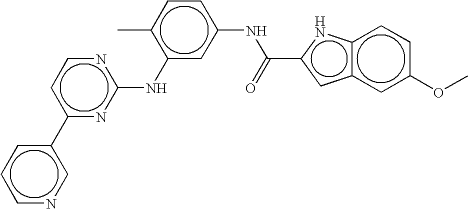 | 451.2 | ¹H NMR (400 MHz, d₆-DMSO) δ 11.5 (s, 1H), 10.1 (s, 1H), 9.27 (s, 1H), 8.99 (s, 1H), 8.69 (d, J = 6.0 Hz, 1H), 8.51 (d, J = 5.2 Hz, 1H), 8.48 (d, J = 8.0 Hz, 1H), 8.08 (s, 1H), 7.52 (m, 2H), 7.43 (d, J = 5.2 Hz, 1H), 7.23 (d, J = 8.4 Hz, 1H), 7.12 (s, 1H), 6.87 (dd, J = 9.6, 2.0 Hz, 1H), 2.23 (s, 3H). |
| A-8 | 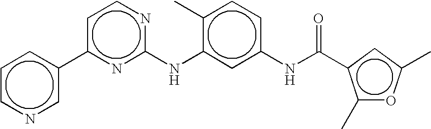 | 400.2 | ¹H NMR (400 MHz, d₆-DMSO) δ 9.56 (s, 1H), 9.30 (s, 1H), 8.96 (s, 1H), 8.73 (s, 1H), 8.57 (d, J = 7.6 Hz, 1H), 8.52 (d, J = 6.4 Hz, 1H), 8.04 (s, 1H), 7.59 (m, 1H), 7.43 (d, J = 5.2 Hz, 1H), 7.37 (d, J = 8.0 Hz, 1H) 7.17 (d, J = 8.0 Hz, 1H), 6.65 (s, 1H), 2.25 (s, 3H), 2.21 (s, 3H). |
| A-9 | 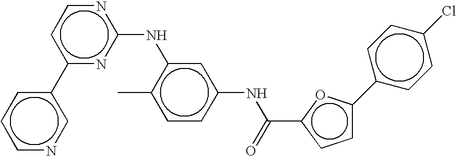 | 482.2 | ¹H NMR (400 MHz, d₆-DMSO) δ 10.2 (s, 1H), 9.28 (s, 1H), 8.99 (s, 1H), 8.69 (d, J = 4.4 Hz, 1H), 8.51 (d, J = 4.8 Hz, 1H), 8.47 (d, J = 8.0 Hz, 1H), 8.01 (m, 3H), 7.83 (d, J = 8.4 Hz, 1H), 7.57 (m, 2H), 7.44 (d, J = 5.2 Hz, 1H), 7.39 (d, J = 3.6 Hz, 1H), 7.23 (m, 2H), 2.23 (s, 3H). |
| A-10 | 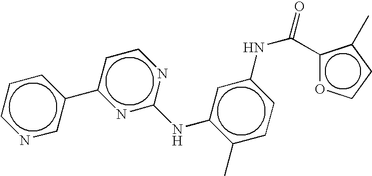 | 386.2 | |
| A-11 | 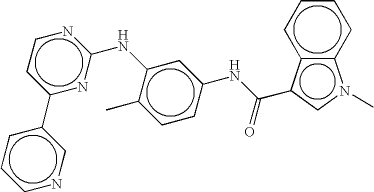 | 435.2 | |
| A-12 | 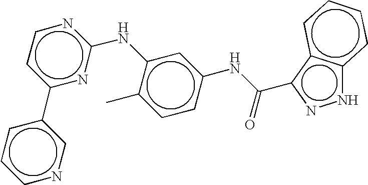 | 422.2 | |
| A-13 | 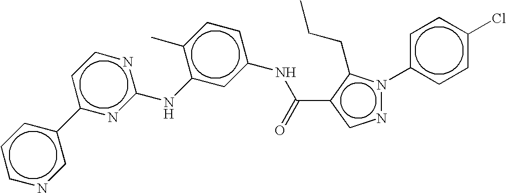 | 524.2 | |
| A-14 | 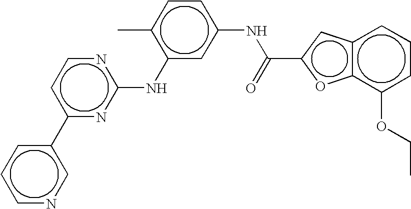 | 466.2 | |
| A-15 | 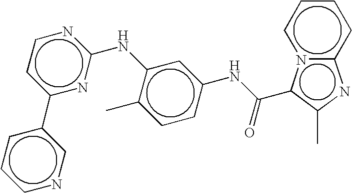 | 436.2 | |
| A-16 | 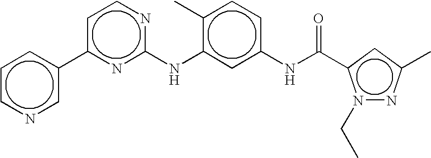 | 414.3 | |
| A-17 | 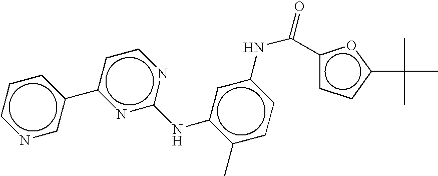 | 428.3 | |
| A-18 | 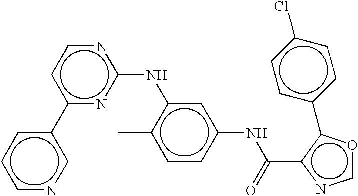 | 483.1 | |
| A-19 | 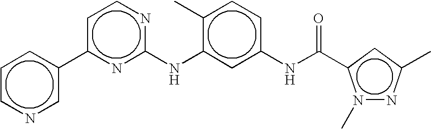 | 400.2 | |
| A-20 | 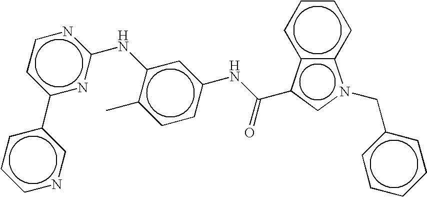 | 511.2 | |
| A-21 | 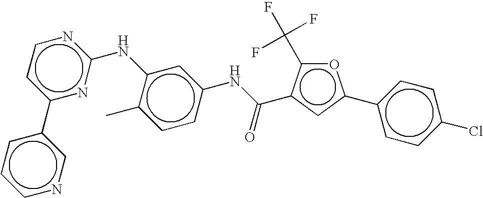 | 550.2 | |
| A-22 | 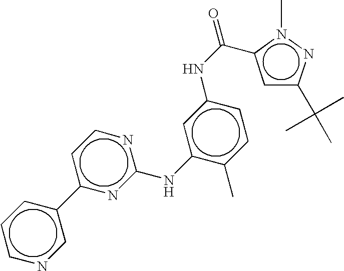 | 442.3 | |
| A-23 | 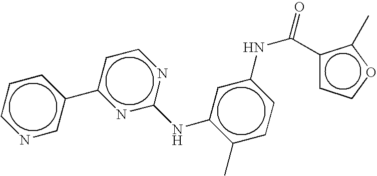 | 386.2 | |
| A-24 | 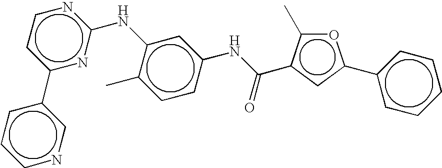 | 462.2 | |
| A-25 | 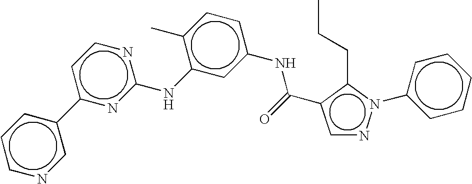 | 490.3 | |
| A-26 | 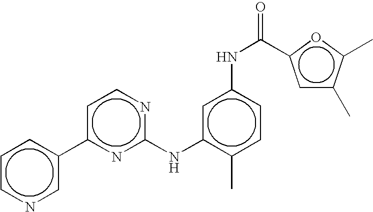 | 400.2 | |
| A-27 | 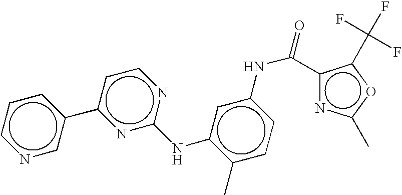 | 455.2 | |
| A-28 | 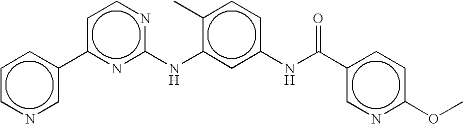 | 413.2 | |
| A-29 | 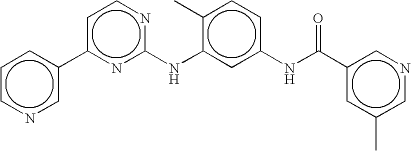 | 397.2 | |
| A-30 | 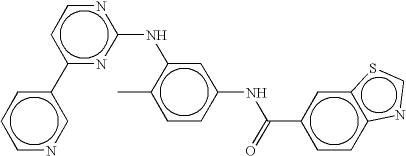 | 439.2 | |
| A-31 | 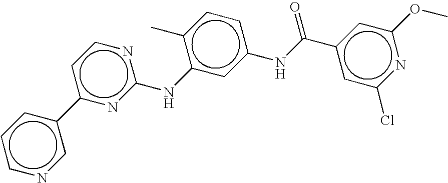 | 447.1 | |
| A-32 | 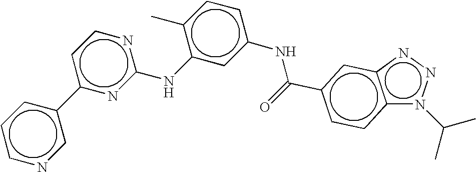 | 465.3 | |
| A-33 | 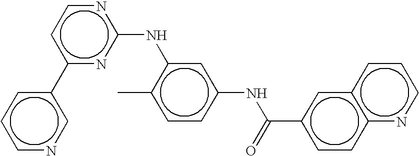 | 433.17 | |
| A-34 | 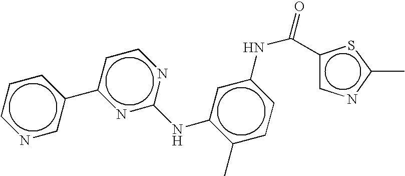 | 403.2 | ¹H NMR (400 MHz, d₆-acetone) δ 9.63 (s, 1H), 9.5 (s, 1H), 8.9 (m, 1H), 8.88 (d, J = 4.5 Hz, 1H), 8.62 (d, J = 5.3 Hz, 1H), 8.56 (s, 1H), 8.48 (m, 2H), 8.33 (s, 1H), 7.81 (dd, J = 7.9, 5.2 Hz, 1H), 7.58 (d, J = 5.3 Hz, 1H), 7.41 (dd, J = 8.3, 1.9 Hz, 1H), 7.26 (d, J = 8.2 Hz, 1H), 2.73 (s, 3H), 2.38 (s, 3H). |
| A-35 | 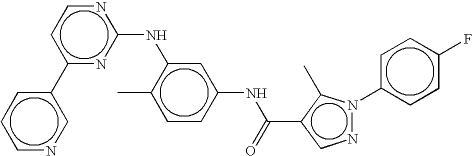 | 480.3 | |
| A-36 | 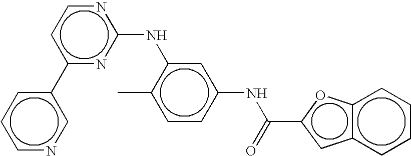 | 422.2 | |
| A-37 | 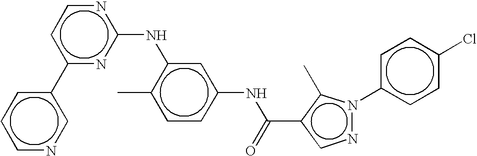 | 496.2 | ¹H NMR (400 MHz, d₆-DMSO) δ 9.85 (s, 1H), 9.32 (m, 1H), 9.01 (s, 1H), 8.72-8.74 (m, 1H), 8.57-8.64 (m, 1H), 8.54 (d, J = 5.1 Hz, 1H), 8.35 (s, 1H), 8.09 (s, 1H), 7.58-7.66 (m, 4H), 7.46 (d, J = 5.2 Hz, 1H), 7.40-7.44 (m, 1H), 7.20 (d, J = 8.2 Hz, 1H), 2.56 (s, 3H), 2.23 (s, 3H). |
| A-38 | 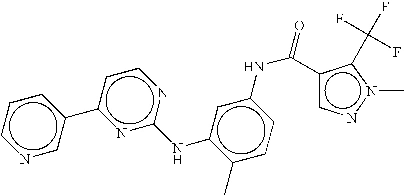 | 454.2 | ¹H NMR (400 MHz, d₆-DMSO) δ 10.1 (s, 1H), 9.33 (s, 1H), 9.09 (s, 1H), 8.79-8.83 (m, 1H), 8.71-8.76 (m, 1H), 8.57 (d, J = 6.1 Hz, 1H), 8.53 (s, 1H), 8.03 (m, 1H), 7.69-7.74 (m, 1H), 7.50 (d, J = 5.1 Hz, 1H), 7.33-7.37 (m, 1H), 7.21 (d, J = 8.3 Hz, 1H), 3.99 (s, 3H), 2.22 (s, 3H). |
| A-39 | 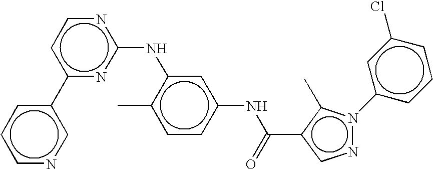 | 496.2 | |
| A-40 | 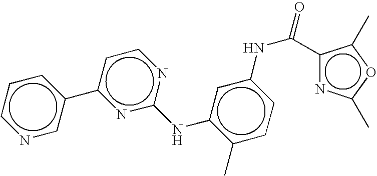 | 401.2 | |
| A-41 | 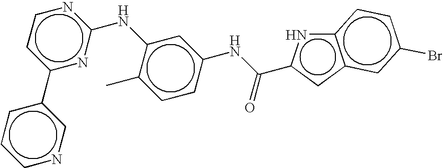 | 499.1 | |
| A-42 | 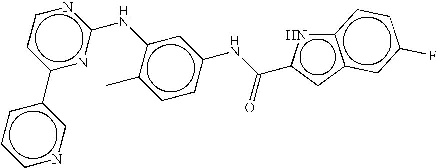 | 439.2 | |
| A-43 | 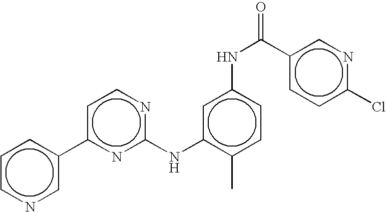 | 417.1 | |
| A-44 | 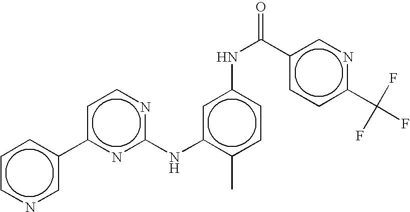 | 451.2 | |
| A-45 | 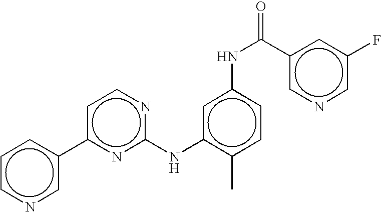 | 401.2 | |
| A-46 | 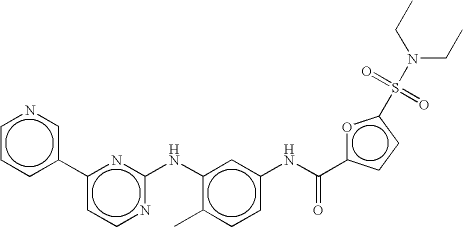 | 455.1 | |
| A-47 | 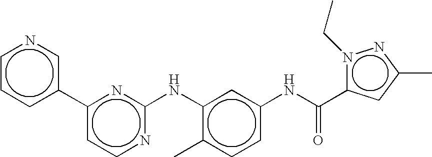 | 414.2 | |
| A-48 | 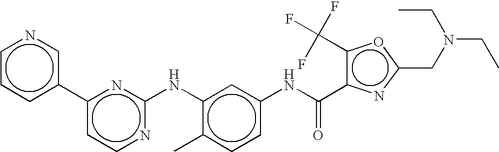 | 526.2 | |
| A-50 | 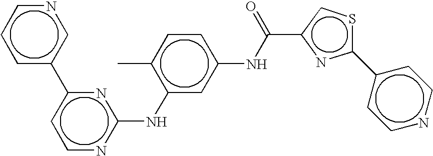 | 466.1 | |
| A-53 | 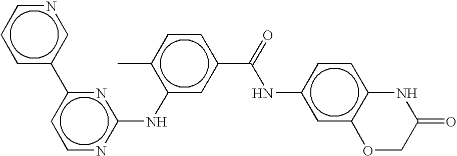 | 453.2 | |
| A-54 | 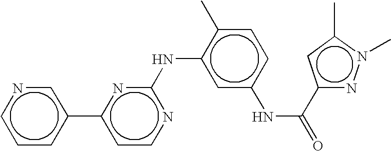 | 400.2 | |
| A-55 | 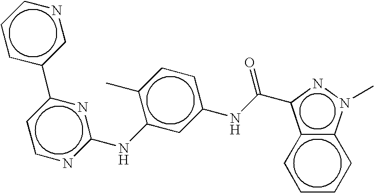 | 436.2 | |
| A-56 | 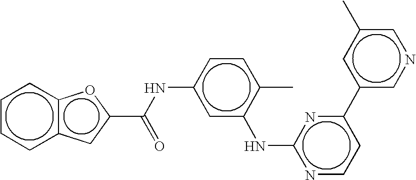 | 436.2 | |
| A-57 | 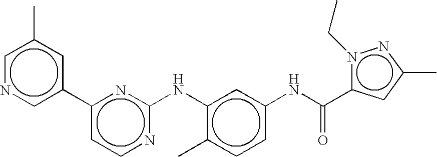 | 428.2 | |
| A-58 | 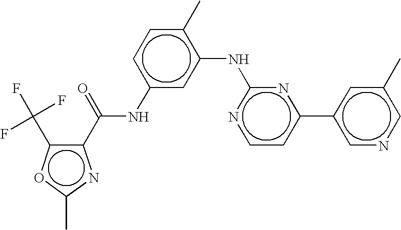 | 469.2 | |
| A-59 | 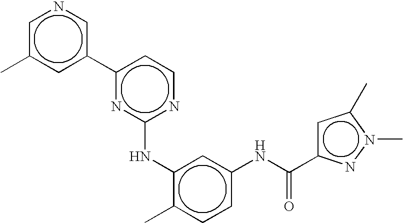 | 414.2 | |
| A-60 | 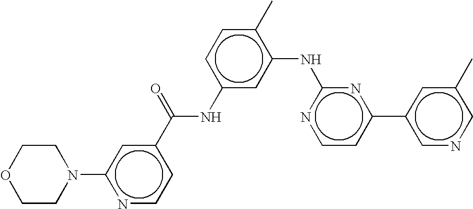 | 482.2 | |
| A-61 | 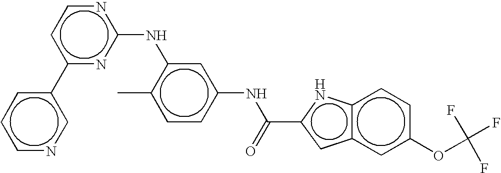 | 505.2 | |
| A-62 | 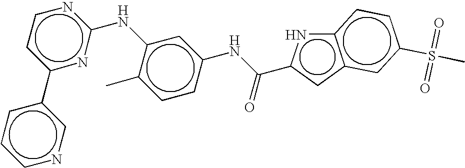 | 499.2 | |
| A-63 | 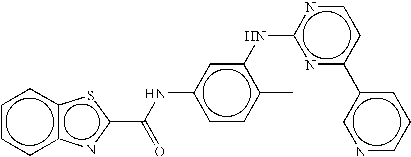 | 439.1 | |
| A-64 | 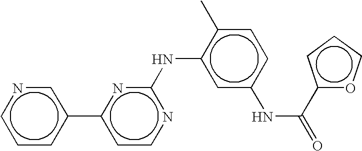 | 372.2 | |
| A-65 | 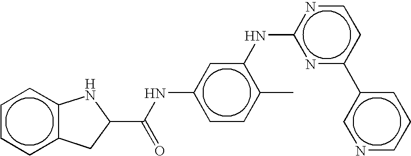 | 423.2 | |
| A-66 | 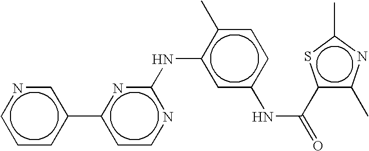 | 417.2 | |
| A-67 | 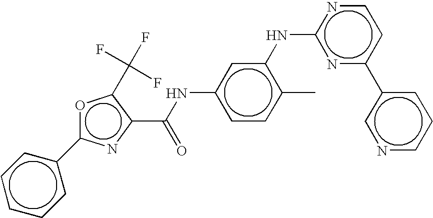 | 517.2 | |
| A-68 | 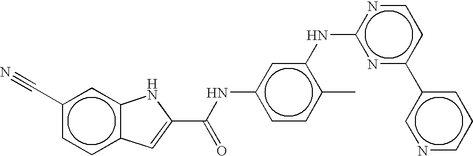 | 446.2 | |
| A-69 | 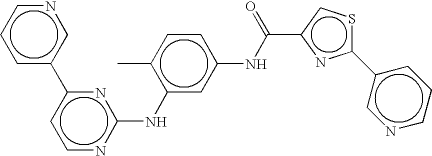 | 466.1 | |
| A-70 | 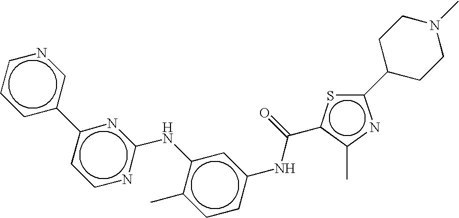 | 500.2 | |
| A-71 | 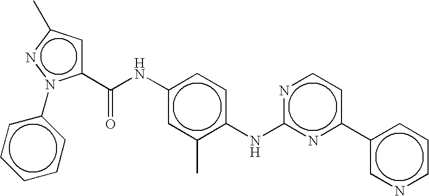 | 462.2 | |
| H-72 | 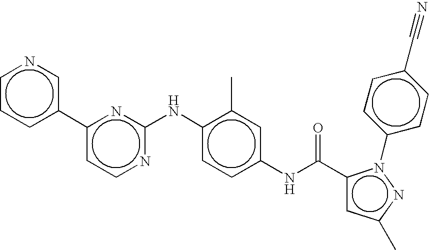 | 487.2 | ¹H NMR (400 MHz, d₆-DMSO) δ 10.1 (s, 1H), 9.23 (s, 1H), 8.9 (s, 1H), 8.67 (d, J = 4.8 Hz, 1H), 8.49 (m, 2H), 8.09 (s, 1H), 8.02 (d, J = 8.4 Hz, 2H), 7.89 (d, J = 8.4 Hz, 2H), 7.54 (m, 1H), 7.43 (m, 1H), 7.39 (d, J = 5.2 Hz, 1H), 7.20 (m, 1H), 6.83 (s, 1H), 2.43 (s, 3H), 2.2 (s, 3H). |
| A-73 | 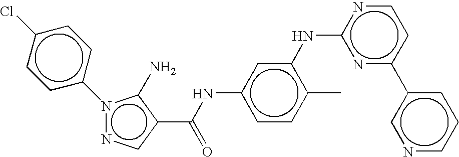 | 497.2, 499.1 | ¹H NMR (400 MHz, d₆-DMSO) δ 9.61 (s, 1H), 9.31 (s, 1H), 8.83 (s, 1H), 8.66 (d, J = 4.4 Hz, 1H), 8.50 (d, J = 5.2 Hz, 2H), 8.17 (s, 1H), 8.10 (s, 1H), 7.57 (m, 3H), 7.40 (d, J = 4.8 Hz, 1H), 7.31 (d, J = 8.0 Hz, 1H), 7.18 (d, J = 7.6 Hz, 1H), 2.21 (s, 3H). |
| A-74 | 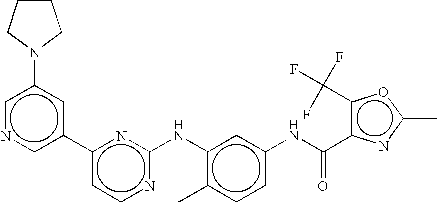 | 524.2 | ¹H NMR (400 MHz, d₆-DMSO) δ 10.5 (s, 1H), 9.09 (s, 1H), 8.57 (m, 2H), 8.14 (s, 1H), 8.09 (s, 1H), 7.87 (s, 1H), 7.51 (d, J = 6.8 Hz, 2H), 7.21 (d, J = 8.0 Hz, 1H), 2.61 (s, 3H), 2.55 (m, 4H), 2.22 (s, 3H), 1.92 (m, 4H). |
| A-75 | 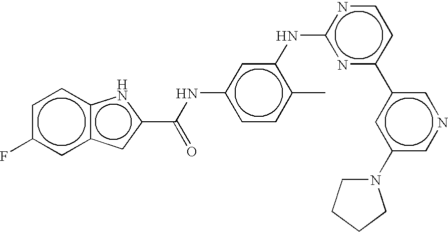 | 508.3 | |
| A-76 | 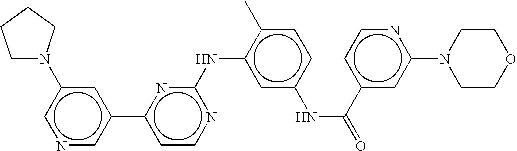 | 537.3 | ¹H NMR (400 MHz, d₆-DMSO) δ 10.3 (s, 1H), 9.11 (s, 1H), 8.60 (s, 2H), 8.28 (d, J = 5.2 Hz, 1H), 8.15 (s, 1H), 8.11 (s, 1H), 7.97 (m, 1H), 7.54 (d, J = 5.2 Hz, 1H), 7.45 (d, J = 8 Hz, 1H), 7.25 (m, 2H), 7.13 (d, J = 5.2 Hz, 1H), 3.72 (m, 2H), 3.32 (m, 2H), 2.51 (m, 8H), 2.24 (s, 3H), 1.89 (m, 4H). |
| A-77 | 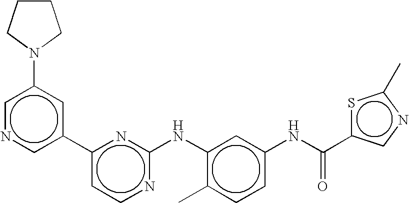 | 472.2 | |
| A-78 | 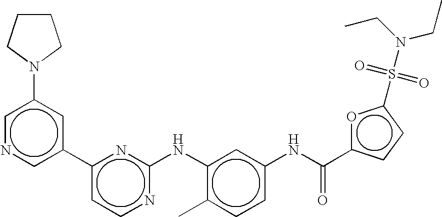 | 576.3 | |
| A-79 | 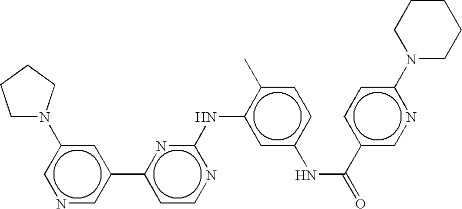 | 535.3 | |
| A-80 | 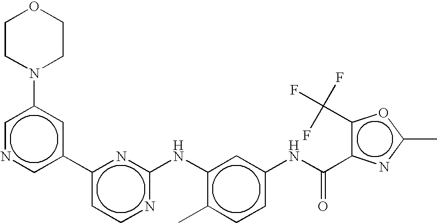 | 539.9 | ¹H NMR (400 MHz, d₄-MeOH) δ 8.67 (d, J = 1.6 Hz, 1H), 8.46 (d, J = 5.3 Hz, 1H), 8.34 (d, J = 2.8 Hz, 1H), 8.16 (d, J = 2.2 Hz, 1H), 8.16 (d, J = 2.2 Hz, 1H), 8.07 (dd, J = 2.8, 1.8 Hz, 1H), 7.46 (dd, J = 8.2, 2.2 Hz, 1H), 7.37 (d, J = 5.3 Hz, 1H), 7.27 (d, J = 8.3 Hz, 1H), 3.8 (dd, J₁ = J₂ = 4.9 Hz, 4H), 3.23 (dd, J₁ = J₂ = 4.8 Hz, 4H), 2.6 (s, 3H), 2.32 (s, 3H). |
| A-81 | 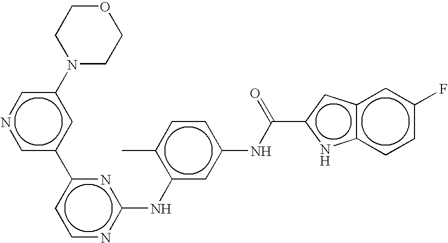 | 524.2 | |
| A-82 | 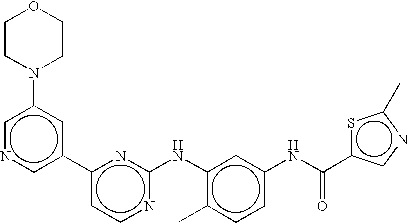 | 488.3 | |
| A-83 | 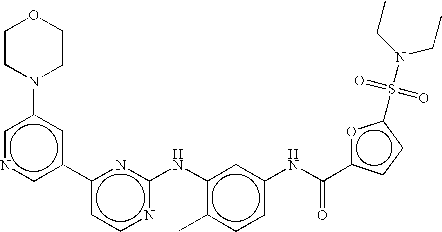 | 592.3 | |
| A-84 | 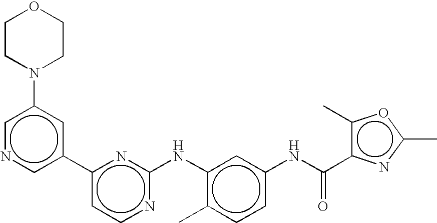 | 486.2 | |
| A-85 | 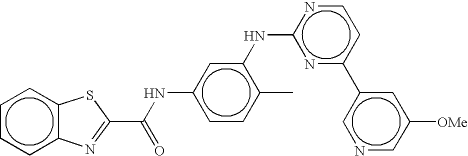 | 469.1 | ¹H NMR (400 MHz, d₆-DMSO) δ 8.95 (s, 1H), 8.89 (s, 1H), 8.81 (s, 1H), 8.67 (m, 1H), 8.52 (d, J = 5.2 Hz, 1H), 8.43 (m, 1H), 8.23 (m, 1H), 8.03 (m, 1H), 7.8 (m, 2H), 7.46 (d, J = 5.2 Hz, 1H), 7.13 (s, 2H), 7.04 (d, J = 8.8 Hz, 1H), 3.73 (s, 3H), 2.19 (s, 3H). |
| A-86 | 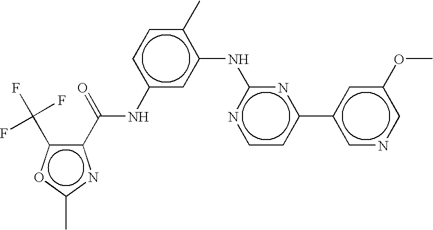 | 485.2 | ¹H NMR (400 MHz, d₆-DMSO) δ 10.54 (s, 1H), 9.09 (s, 1H), 8.89 (s, 1H), 8.54 (d, J = 5.2 Hz, 1H), 8.45 (d, J = 2.4 Hz, 1H), 8.08 (m, 2H), 7.50 (m, 1H), 7.21 (m, 1H), 3.86 (s, 3H), 2.61 (s, 3H), 2.23 (s, 3H). |
| A-87 | 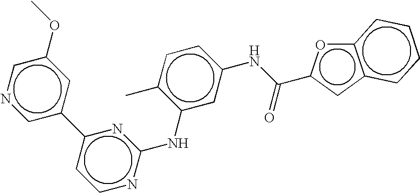 | 452.1 | ¹H NMR (400 MHz, d₆-DMSO) δ 10.5 (s, 1H), 9.08 (s, 1H), 8.91 (d, J = 1.6 Hz, 1H), 8.55 (d, J = 5.2 Hz, 1H), 8.43 (d, J = 2.8 Hz, 1H), 8.16 (m, 1H), 8.07 (m, 1H), 7.83 (m, 1H), 7.76 (m, 1H), 7.72 (m, 1H), 7.51 (m, 2H), 7.38 (m, 1H), 7.24 (d, J = 8.0 Hz, 1H), 3.85 (s, 3H), 2.25 (s, 3H). |
| A-88 | 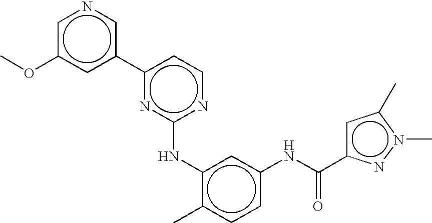 | 430.2 | |
| A-89 | 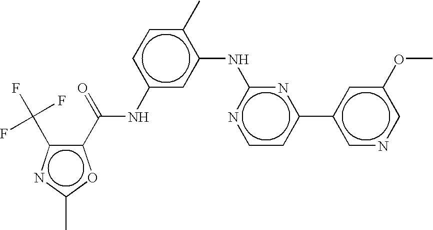 | 485.2 | |
| A-90 | 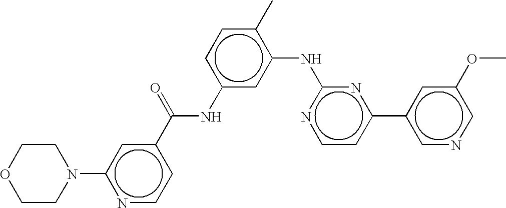 | 498.2 | |
| A-91 | 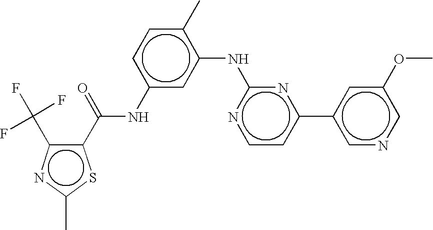 | 501.1 | |
| A-92 | 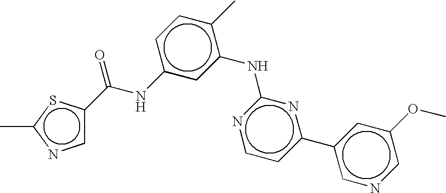 | 433.1 | ¹H NMR (400 MHz, d₆-DMSO) δ 10.32 (s, 1H), 9.07 (s, 1H), 8.92 (d, J = 1.6 Hz, 1H), 8.55 (d, J = 5.2 Hz, 1H), 8.46 (d, J = 2.8 Hz, 1H), 8.05 (m, 3H), 7.51 (d, J = 5.2 Hz, 1H), 7.42 (dd, J = 8.4, 2.0 Hz, 1H), 7.22 (d, J = 8.4 Hz, 1H), 3.86 (s, 3H), 2.71 (s, 3H), 2.24 (s, 3H). |
| A-93 | 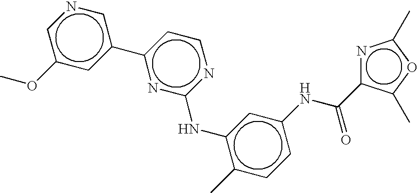 | 431.2 | |
| A-94 | 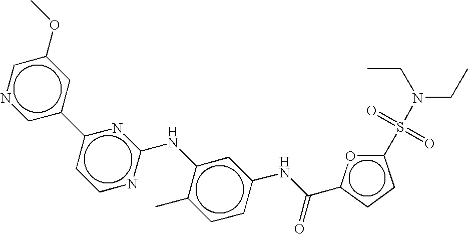 | 537.2 | ¹H NMR (400 MHz, d₆-DMSO) δ 10.37 (s, 1H), 9.07 (s, 1H), 8.90 (d, J = 1.6 Hz, 1H), 8.54 (d, J = 5.2 Hz, 1H), 8.44 (d, J = 2.8 Hz, 1H), 8.04 (m, 2H), 7.51 (m, 2H), 7.45 (dd, J = 8.0, 2.4 Hz, 1H), 7.30 (d, J = 3.6 Hz, 1H), 7.23 (m, 1H), 3.85 (s, 3H), 3.29 (q, J = 7.2 Hz, 4H), 2.24 (s, 3H), 1.09 (t, J = 7.2 Hz, 6H). |
| A-95 | 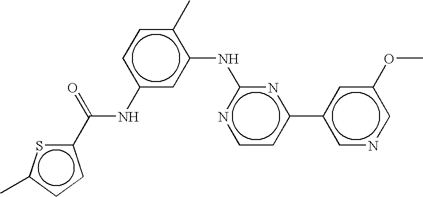 | 432.1 | |
| A-96 | 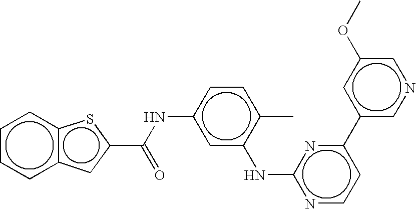 | 468.1 | |
| A-97 | 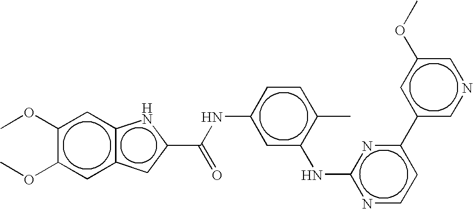 | 511.2 | |
| A-98 | 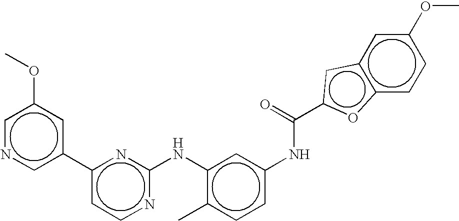 | 482.2 | |
| A-99 | 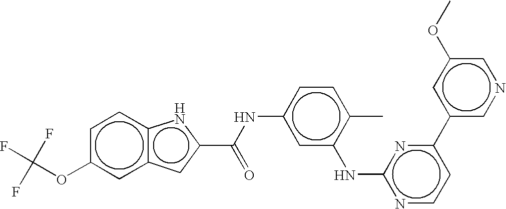 | 535.2 | |
| A-100 | 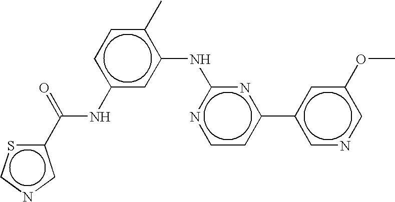 | 419.1 | |
| A-101 | 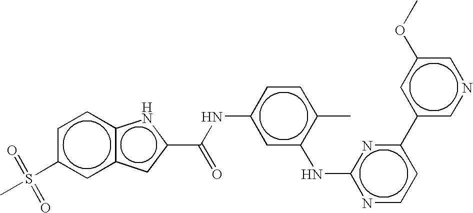 | 529.2 | |
| A-102 | 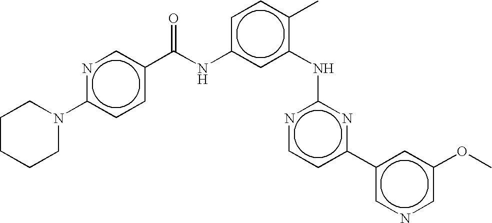 | 496.2 | |
| A-103 | 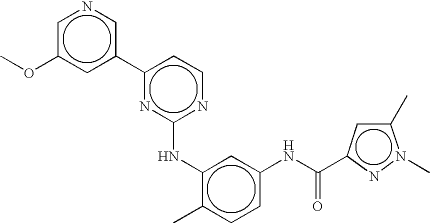 | 430.2 | |
| A-104 | 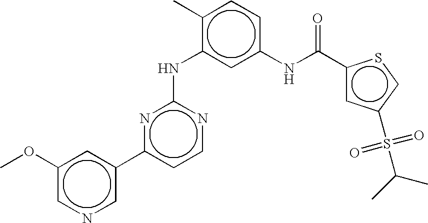 | 524.0 | |
| A-105 | 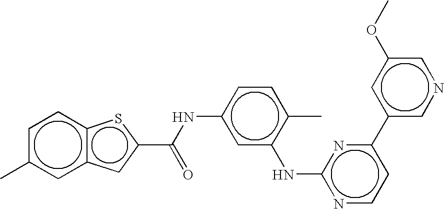 | 482.2 | |
| A-106 | 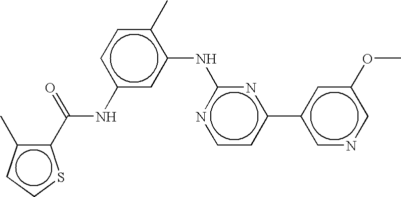 | 432.2 | |
| A-107 | 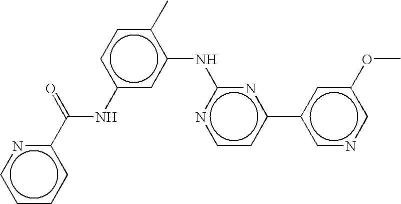 | 413.2 | |
| A-108 | 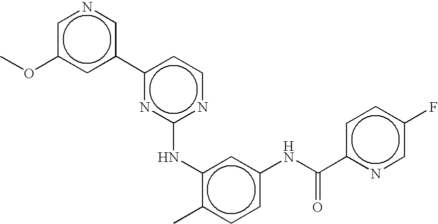 | 431.1 | |
| A-109 | 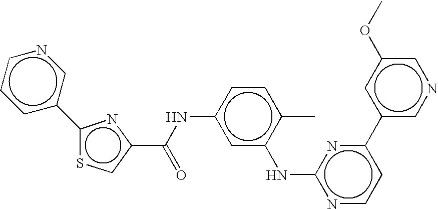 | 496.1 | |
| A-110 | 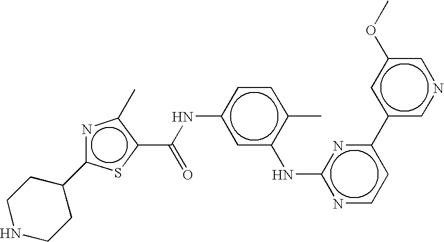 | 516.2 | |
| A-111 | 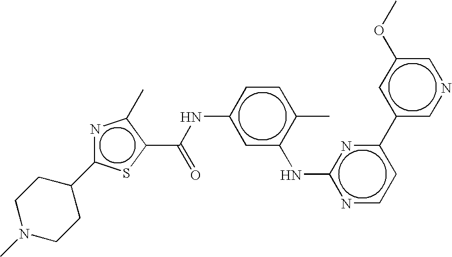 | 530.2 | |
| A-112 | 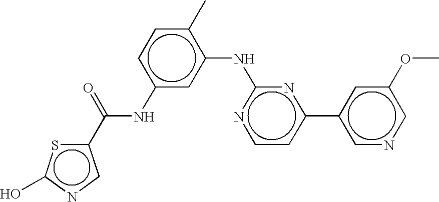 | 435.1 | |
| A-113 | 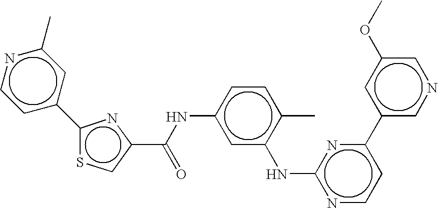 | 510.2 | |
| A-114 | 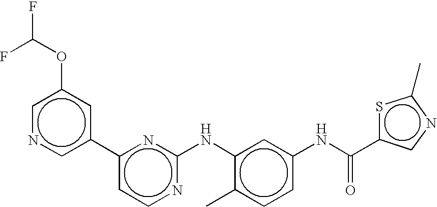 | 469.2 | ¹H NMR (400 MHz, d₆-DMSO) δ 10.03 (s, 1H), 9.18 (s, 1H), 9.06 (s, 1H), 8.61 (s, 1H), 8.5 (d, J = 4.9 Hz, 1H), 8.5 (d, J = 4.9 Hz, 1H), 8.42 (s, 1H), 8.29 (s, 1H), 8.02 (s, 1H), 7.51 (d, J = 5.1 Hz, 1H), 7.41 (d, J = 7.9 Hz, 1H), 7.34 (t, J = 73.2 Hz, 1H), 7.22 (d, J = 8.4 Hz, 1H), 2.7 (s, 3H), 2.23 (s, 3H). |
| A-115 | 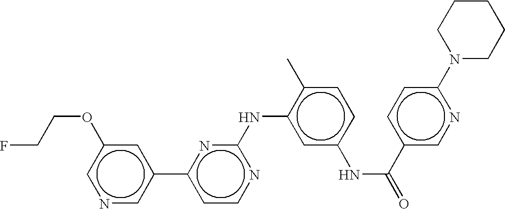 | 528.3 | |
| A-116 | 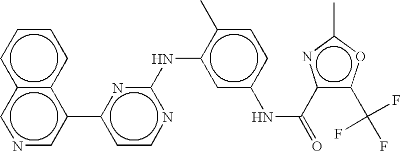 | 505.2 | ¹H NMR (400 MHz, d₆-DMSO) δ 10.5 (s, 1H), 9.48 (s, 1H), 9.12 (s, 1H), 8.72 (s, 1H), 8.58 (d, J = 4.8 Hz, 1H), 8.40 (d, J = 8.8 Hz, 1H), 8.26 (d, J = 7.6 Hz, 1H), 8.03 (s, 1H), 7.84 (t, J = 7.2 Hz, 1H), 7.74 (t, J = 8.0 Hz, 1H), 7.48 (d, J = 8.0 Hz, 1H), 7.17 (m, 2H), 2.63 (s, 3H), 2.23 (s, 3H). |
| A-117 | 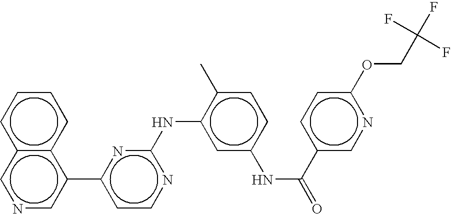 | 531.2 | |
| A-118 | 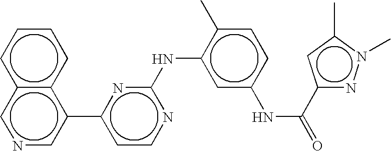 | 450.2 | ¹H NMR (400 MHz, d₆-DMSO) δ 9.82 (s, 1H), 9.46 (s, 1H), 9.08 (s, 1H), 8.72 (s, 1H), 8.57 (d, J = 4.8 Hz, 1H), 8.42 (d, J = 7.7 Hz, 1H), 8.25 (d, J = 8.4 Hz, 1H), 8.03 (s, 1H), 7.86 (t, J = 7.1 Hz, 1H), 7.72 (t, J = 7.9 Hz, 1H), 7.49 (d, J = 8.3 Hz, 1H), 7.13 (m, 2H), 6.55 (s, 1H), 3.85 (s, 3H), 2.32 (s, 3H), 2.21 (s, 3H). |
| A-119 | 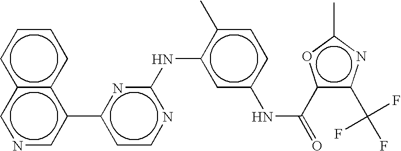 | 505.3 | ¹H NMR (400 MHz, d₆-DMSO) δ 10.5 (s, 1H), 9.47 (s, 1H), 9.12 (s, 1H), 8.72 (s, 1H), 8.57 (d, J = 4.8 Hz, 1H), 8.40 (d, J = 8.8 Hz, 1H), 8.25 (d, J = 8.4 Hz, 1H), 8.04 (m, 1H), 7.84 (t, J = 7.2 Hz, 1H), 7.74 (t, J = 7.2 Hz, 1H), 7.48 (d, J = 8.4 Hz, 1H), 7.17 (m, 2H), 2.63 (s, 3H), 2.55 (s, 3H), 2.23 (s, 3H). |
| A-120 | 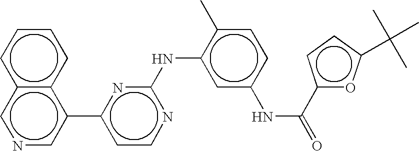 | 478.2 | |
| A-121 | 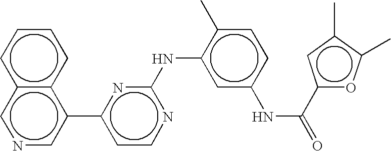 | 450.2 | |
| A-122 | 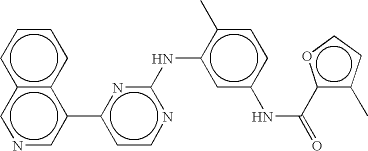 | 436.2 | |
| A-123 | 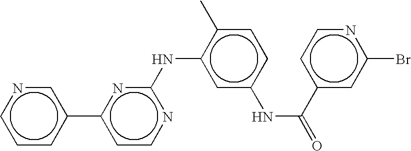 | 461.32 | |
| A-124 | 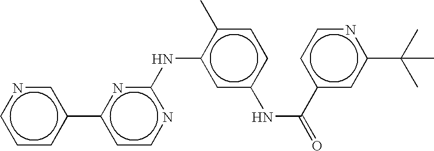 | 438.54 | |
| B-1 | 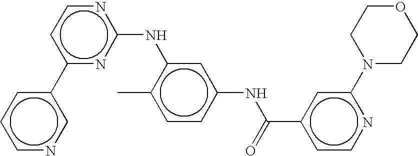 | 468.4 | ¹H NMR (400 MHz, d₆-acetone) δ 9.47 (s, 1H), 9.22 (s, 1H), 8.56 (dd, J = 4.7, 1.6 Hz, 1H), 8.45 (m, 1H), 8.41 (m, 1H), 8.4 (m, 1H), 8.15 (d, J = 5.1 Hz, 1H), 7.87 (s, 1H), 7.37 (m, 2H), 7.3 (d, J = 5.1 Hz, 1H), 7.17 (s, 1H), 7.11 (d, J = 8.2 Hz, 1H), 7.03 (dd, J = 5.1, 1.2 Hz, 1H), 3.63 (t, J = 4.7 Hz, 4H), 3.44 (t, J = 4.7 Hz, 1H), 2.24 (s, 3H). |
| B-2 | 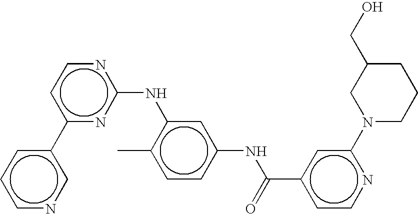 | 496.3 | ¹H NMR (400 MHz, d₆-DMSO) δ 10.4 (s, 1H), 9.33 (s, 1H), 9.04 (s, 1H), 8.75 (d, J = 4.0 Hz, 1H), 8.61 (d, J = 8.0 Hz, 1H), 8.54 (d, J = 5.2 Hz, 1H), 8.18 (d, J = 5.6 Hz, 1H), 8.09 (m, 1H), 7.66 (m, 1H), 7.47 (m, 1H), 7.66 (m, 1H), 7.47 (m, 3H), 7.24 (d, J = 8.4 Hz, 1H), 7.10 (d, J = 5.2 Hz, 1H), 4.29 (d, J = 14.8 Hz, 1H, 4.20 (d, J = 12.4 Hz, 1H), 3.04 (m, 2H), 2.84 (m, 2H), 2.24 (s, 3H), 1.52 (m, 1H), 1.27 (m, 1H). |
| B-3 | 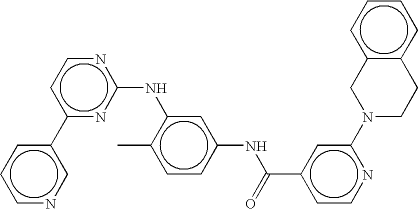 | 514.3 | ¹H NMR (400 MHz, d₆-DMSO) δ 10.4 (s, 1H), 9.34 (s, 1H), 9.04 (s, 1H), 8.76 (m, 1H), 8.62 (d, J = 7.6 Hz, 1H), 8.55 (d, J = 4.8 Hz, 1H), 8.26 (d, J = 5.2 Hz, 1H), 8.11 (s, 1H), 7.66 (m, 1H), 7.47 (m, 2H), 7.43 (s, 1H), 7.24 (m, 5H), 7.13 (d, J = 5.2 Hz, 1H), 4.79 (s, 2H), 3.9 (m, 2H), 2.96 (m, 2H), 2.25 (s, 3H). |
| B-4 | 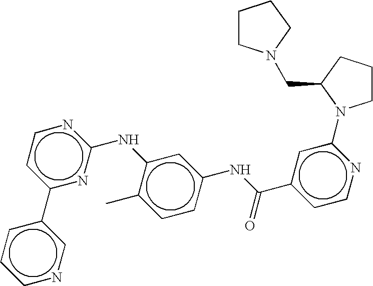 | 535.3 | ¹H NMR (400 MHz, d₆-DMSO) δ 10.38 (s, 1H), 9.88 (bs, 1H), 9.38 (m, 1H), 9.08 (s, 1H), 8.77-8.80 (m, 1H), 8.64-8.69 (m, 1H), 8.56 (d, J = 5.1 Hz, 1H), 8.27 (d, J = 5.3 Hz, 1H), 8.08-8.11 (m, 1H), 7.67-7.72 (m, 1H), 7.44-7.50 (m, 2H), 7.24 (d, J = 8.2 Hz, 1H), 7.15-7.17 (m, 1H), 7.05 (s, 1H), 4.46 (m, 1H), 3.5-4.0 (m, 4H), 3.2-3.4 (m, 4H), 2.24 (s, 3H), 1.80-2.13 (m, 8H). |
| B-5 | 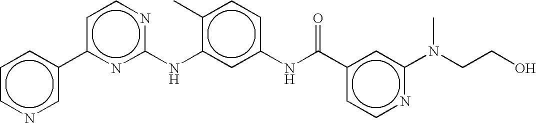 | 456.2 | ¹H NMR (400 MHz, d₆-DMSO) δ 10.5 (s, 1H), 9.29 (s, 1H), 8.99 (s, 1H), 8.71 (d, J = 4.4 Hz, 1H), 8.56 (d, J = 8.4 Hz, 1H), 8.52 (d, J = 4.8 Hz, 1H), 8.09 (s, 2H), 7.61 (m, 1H), 7.44 (m, 2H), 7.25 (d, J = 8.4 Hz, 1H), 7.12 (d, J = 6.0 Hz, 1H), 3.19 (m, 4H), 2.55 (s, 3H), 2.23 (s, 3H). |
| B-6 | 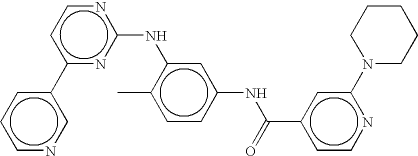 | 466.3 | ¹H NMR (400 MHz, d₆-DMSO) δ 10.4 (s, 1H), 9.31 (s, 1H), 9.02 (s, 1H), 8.73 (s, 1H), 8.56 (m, 2H), 8.18 (m, 1H), 8.09 (m, 1H), 7.62 (m, 1H), 7.46 (m, 2H), 7.25 (m, 1H), 7.07 (m, 1H), 3.64 (m, 6H), 2.23 (s, 3H), 1.61 (m, 4H). |
| B-7 | 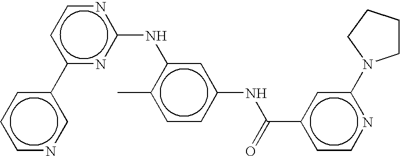 | 452.3 | ¹H NMR (400 MHz, d₄-methanol) δ 9.51 (s, 1H), 8.93 (d, J = 8.1 Hz, 1H), 8.79 (d, J = 4.9 Hz, 1H), 8.55 (d, J = 5.3 Hz, 1H), 8.37 (m, 1H), 8.00 (d, J = 6.7 Hz, 1H), 7.83-7.87 (m, 1H), 7.56 (s, 1H), 7.46 (d, J = 5.3 Hz, 1H), 7.34-7.38 (m, 1H), 7.30 (d, J = 7.7 Hz, 1H), 3.20-3.27 (m, 4H), 2.35 (s, 3H), 2.16-2.22 (m, 4H). |
| B-8 | 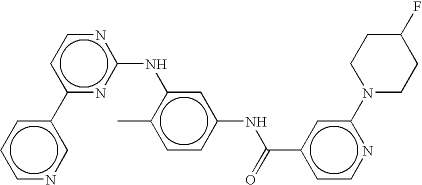 | 484.3 | ¹H NMR (400 MHz, d₆-DMSO) δ 10.4 (s, 1H), 9.32 (s, 1H), 9.02 (s, 1H), 8.74 (d, J = 4.8 Hz, 1H), 8.60 (d, J = 8.4 Hz, 1H), 8.54 (d, J = 5.2 Hz, 1H), 8.23 (d, J = 4.8 Hz, 1H), 8.09 (m, 1H), 7.65 (m, 1H), 7.46 (m, 2H), 7.38 (s, 1H), 7.24 (d, J = 8.4 Hz, 1H), 7.09 (d, J = 5.2 Hz, 1H), 4.94 (m, 1H), 3.53 (m, 4H), 2.24 (s, 3H), 1.96 (m, 2H), 1.74 (m, 2H). |
| B-9 | 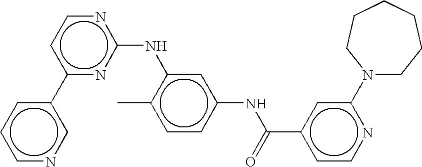 | 480.3 | ¹H NMR (400 MHz, d₄-methanol) δ 9.61 (s, 1H), 9.10 (dt, J = 8.2, 1.7 Hz, 1H), 8.86 (d, J = 4.7 Hz, 1H), 8.57 (d, J = 5.3 Hz, 1H), 8.39 (d, J = 1.7 Hz, 1H), 7.97-8.03 (m, 2H), 7.71 (s, 1H), 7.50 (d, J = 5.3 Hz, 1H), 7.29-7.36 (m, 3H), 3.80 (t, J = 5.9 Hz, 4H), 2.35 (s, 3H), 1.90-1.98 (m, 4H), 1.66-1.72 (m, 4H). |
| B-10 | 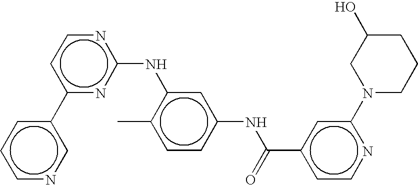 | 482.3 | ¹H NMR (400 MHz, d₆-DMSO) δ 10.3 (s, 1H), 9.31 (s, 1H), 9.0 (s, 1H), 8.72 (m, 1H), 8.53 (d, J = 5.6 Hz, 2H), 8.30 (s, 1H), 8.20 (d, J = 4.8 Hz, 1H), 8.09 (s, 1H), 7.58 (m, 1H), 7.45 (d, J = 5.2 Hz, 2H), 7.32 (s, 1H), 7.24 (d, J = 8 Hz, 1H), 7.04 (d, J = 5.2 Hz, 1H), 4.18 (m, 1H), 3.64 (m, 2H), 3.14 (m, 2H), 2.24 (s, 3H), 1.92 (m, 2H), 1.76 (m, 2H). |
| B-11 | 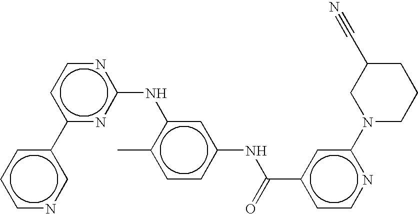 | 491.2 | ¹H NMR (400 MHz, d₆-DMSO) δ 10.3 (s, 1H), 9.33 (s, 1H), 9.03 (s, 1H), 8.75 (d, J = 4.4 Hz, 1H), 8.60 (d, J = 8.0 Hz, 1H), 8.55 (d, J = 4.8 Hz, 1H), 8.26 (d, J = 5.2 Hz, 1H), 8.09 (s, 1H), 7.65 (m, 1H), 7.45 (m, 2H), 7.36 (s, 1H), 7.24 (d, J = 8.4 Hz, 1H), 7.10 (d, J = 4.8 Hz, 1H), 3.95 (m, 1H), 3.67 (m, 2H), 3.10 (m, 2H), 2.24 (s, 3H), 1.96 (m, 2H), 1.71 (m, 2H). |
| B-12 | 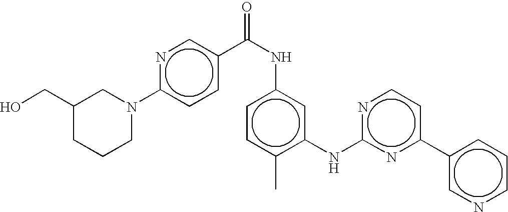 | 496.2 | ¹H NMR (400 MHz, d₄-methanol) δ 9.53 (s, 1H), 8.97 (m, 1H), 8.80 (d, J = 6.4 Hz, 1H), 8.59 (d, J = 2.0 Hz, 1H), 8.54 (d, J = 5.2 Hz, 1H), 8.38 (dd, J = 9.6 Hz, 2.4 Hz, 1H), 8.31 (d, J = 1.6 Hz, 1H), 7.88 (m, 1H), 7.47 (d, J = 5.6 Hz, 1H), 7.31 (m, 3H), 4.25 (m, 2H), 3.59 (m, 1H), 3.49 (m, 1H), 3.34 (m, 1H), 3.17 (m, 1H), 2.33 (s, 3H), 1.93 (m, 3H), 1.69 (m, 1H), 1.46 (m, 1H). |
| B-13 | 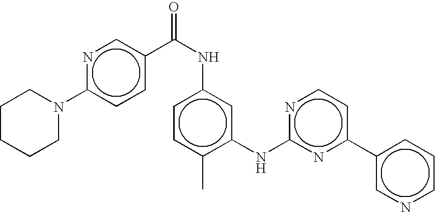 | 466.2 | |
| B-14 | 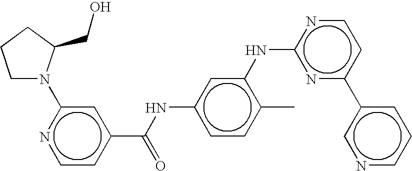 | 482.3 | |
| B-15 | 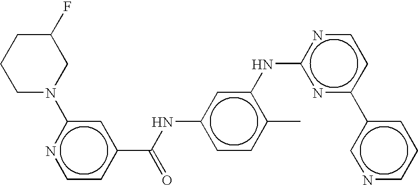 | 484.2 | |
| B-16 | 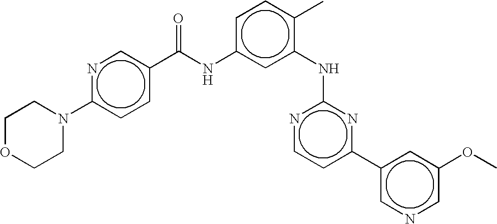 | 498.2 | |
| B-17 | 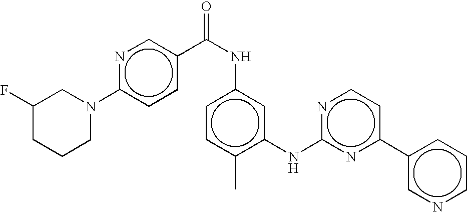 | 484.2 | |
| C-1 | 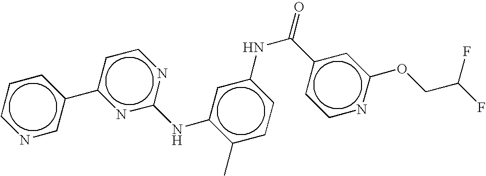 | 464.2 | ¹H NMR (400 MHz, d₆-DMSO) δ 10.43 (s, 1H), 9.33 (s, 1H), 9.03 (s, 1H), 8.76 (d, J = 4.8 Hz, 1H), 8.64 (d, J = 7.6 Hz, 1H), 8.55 (d, J = 5.2 Hz, 1H), 8.36 (d, J = 5.2 Hz, 1H), 8.12 (s, 1H), 7.67 (s, 1H), 7.48 (m, 4H), 7.24 (d, J = 8.4 Hz, 1H), 6.42 (d, J = 58.4 Hz, 1H), 4.64 (dt, J = 14.8 Hz, 3.2 Hz, 2H), 2.24 (s, 3H). |
| C-2 | 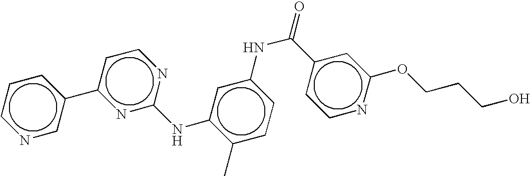 | 457.3 | ¹H NMR (400 MHz, d₆-DMSO) δ 10.40 (s, 1H), 9.41 (d, J = 1.44 Hz, 1H), 9.14 (s, 1H), 8.83-8.88 (m, 2H), 8.60 (d, J = 5.2 Hz, 1H), 8.15 (s, 1H), 7.83-7.88 (m, 1H), 7.53 (d, J = 5.2 Hz, 1H), 7.41-7.49 (m, 2H), 7.32 (s, 1H), 7.23 (d, J = 8.3 Hz, 1H), 4.38 (t, J = 6.5 Hz, 2H), 3.57 (t, J = 6.2 Hz, 2H), 2.24 (s, 3H), 1.85-1.93 (m, 2H). |
| C-3 | 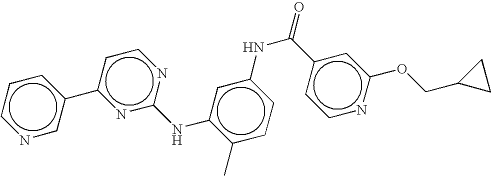 | 453.2 | ¹H NMR (400 MHz, d₆-DMSO) δ 10.38 (s, 1H), 9.32 (s, 1H), 8.75 (d, J = 5.2 Hz, 1H), 8.62 (d, J = 7.2 Hz, 1H), 8.55 (d, J = 4.8 Hz, 1H), 8.30 (d, J = 4.8 Hz, 1H), 8.12 (s, 1H), 7.65 (m, 1H), 7.47 (d, J = 5.6 Hz, 2H), 7.41 (d, J = 4.8 Hz, 1H), 7.31 (s, 1H), 7.23 (d, J = 8.0 Hz, 1H), 4.15 (d, J = 7.2 Hz, 2H), 2.23 (s, 3H), 1.26 (m, 1H), 0.56 (d, J = 7.2 Hz, 2H), 0.34 (d, J = 4.0 Hz, 2H). |
| C-4 | 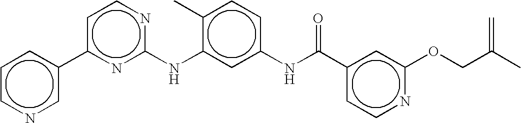 | 453.2 | |
| C-5 | 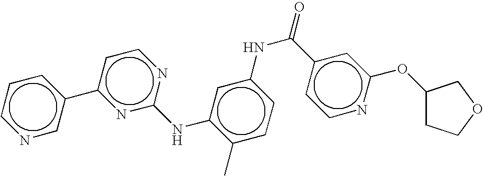 | 480.3 | |
| C-6 | 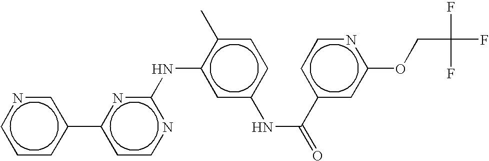 | 481.2 | |
| C-7 | 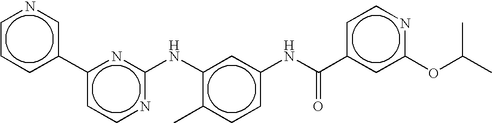 | 441.3 | |
| C-8 | 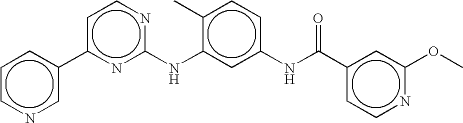 | 413.2 | ¹H NMR (400 MHz, d₆-DMSO) δ 10.5 (s, 1H), 9.23 (s, 1H), 8.84 (s, 1H), 8.67 (s, 1H), 8.55 (d, J = 6.0 Hz, 1H), 8.49 (d, J = 4.8 Hz, 1H), 8.29 (d, J = 4.8 Hz, 1H), 8.08 (s, 1H), 7.62 (m, 1H), 7.39 (m, 2H), 7.07 (m, 2H), 2.55 (s, 3H), 2.21 (s, 3H). |
| C-9 | 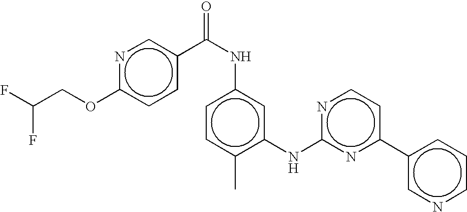 | 463.2 | ¹H NMR (400 MHz, d₄-Methanol) δ 9.57 (d, J = 2.0 Hz, 1H), 9.05 (d, J = 8.0 Hz, 1H), 8.81 (m, 1H), 8.77 (d, J = 2.0 Hz, 1H), 8.55 (d, J = 5.6 Hz, 1H), 8.34 (s, 1H), 8.26 (dd, J = 2.8, 8.8 Hz, 1H), 7.93 (m, 1H), 7.48 (d, J = 5.6 Hz, 1H), 7.27 (m, 2H), 6.97 (m, 1H), 6.23 (tt, J = 55.2, 4 Hz, 1H), 4.62 (td, J = 14, 4 Hz, 2H), 2.33 (s, 3H). |
| C-10 | 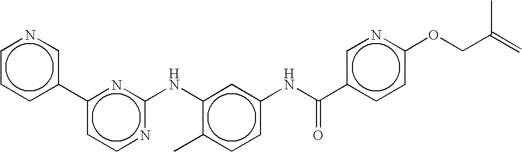 | 453.2 | ¹H NMR (400 MHz, d₆-DMSO) δ 10.2 (s, 1H), 9.33 (s, 1H), 9.01 (s, 1H), 8.76 (s, 2H), 8.63 (d, J = 8.0 Hz, 1H), 8.54 (d, J = 5.2 Hz, 1H), 8.25 (d, J = 8.8 Hz, 1H), 8.09 (s, 1H), 7.66 (m, 1H), 7.45 (m, 2H), 7.21 (d, J = 8.4 Hz, 1H), 6.98 (d, J = 8.4 Hz, 1H), 5.02 (s, 1H), 4.94 (s, 1H), 2.22 (s, 3H), 1.78 (s, 3H). |
| C-11 | 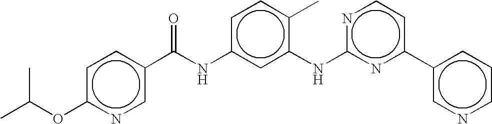 | 441.2 | ¹H NMR (400 MHz, d₆-DMSO) δ 10.2 (s, 1H), 9.29 (s, 1H), 8.97 (s, 1H), 8.75 (m, 1H), 8.69 (m, 1H), 8.18 (m, 1H), 8.10 (m, 2H), 7.44 (m, 1H), 7.21 (d, J = 8.4 Hz, 1H), 6.83 (m, 2H), 5.31 (m, 1H), 2.55 (s, 6H), 2.22 (s, 3H). |
| C-12 | 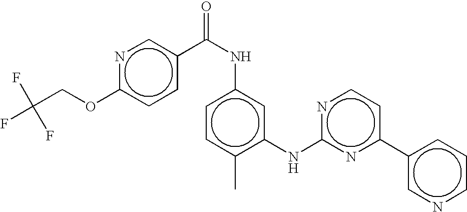 | 481.1 | ¹H NMR (400 MHz, d₆-DMSO) δ 10.3 (s, 1H), 9.32 (s, 1H), 9.03 (s, 1H), 8.80 (d, J = 2.4 Hz, 1H), 8.74 (d, J = 4.8 Hz, 1H), 8.58 (m, 1H), 8.54 (d, J = 5.2 Hz, 1H), 8.33 (dd, J = 8.8, 2.4 Hz, 1H), 7.61 (m, 1H), 7.45 (m, 2H), 7.23 (d, J = 8.4 Hz, 1H), 7.15 (d, J = 8.8 Hz, 1H), 5.10 (q, J = 8.8 Hz, 2H), 2.24 (s, 3H). |
| D-1 | 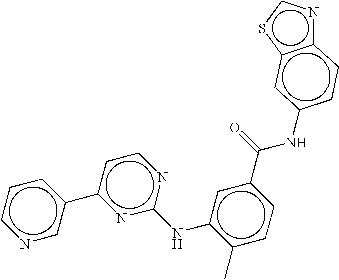 | 439.2 | ¹H NMR (400 MHz, d₆-DMSO) δ 10.46 (s, 1H), 9.29 (m, 1H), 9.16 (s, 1H), 8.69 (m, 1H), 8.55 (m, 1H), 8.49 (m, 1H), 8.30 (m, 1H), 8.05 (d, J = 8.8 Hz, 1H), 7.83 (m, 1H), 7.76 (m, 1H), 7.54 (m, 1H), 7.45 (m, 2H), 2.35 (s, 3H). |
| D-2 | 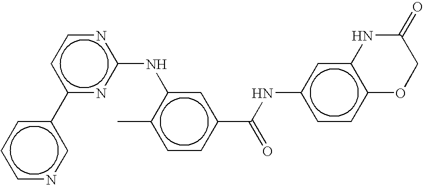 | 453.2 | ¹H NMR (400 MHz, d₆-DMSO) δ 10.78 (s, 1H), 10.15 (s, 1H), 9.29 (d, J = 1.7 Hz, 1H), 9.18 (s, 1H), 8.74 (dd, J = 1.4, 4.9 Hz, 1H), 8.52-8.58 (m, 2H), 8.23 (d, J = 1.3 Hz, 1H), 7.71 (dd, J = 1.7, 7.9 Hz, 1H), 7.59-7.64 (m, 1H), 7.54 (d, J = 2.4 Hz, 1H), 7.49 (d, J = 5.2 Hz, 1H), 7.40 (d, J = 8.1 Hz, 1H), 7.23 (dd, J = 2.4, 8.7 Hz, 1H), 6.92 (d, J = 8.7 Hz, 1H), 4.54 (s, 2H), 2.34 (s, 3H). |
| D-3 | 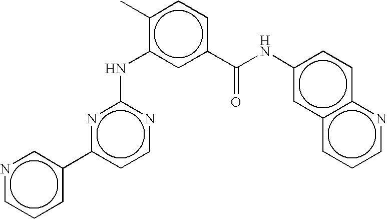 | 433.3 | ¹H NMR (400 MHz, d₆-DMSO) δ 10.65 (s, 1H), 9.30 (s, 1H), 9.19 (s, 1H), 8.96 (m, 1H), 8.69 (m, 2H), 8.53 (m, 2H), 8.33 (s, 1H), 8.18 (m, 1H), 7.81 (m, 1H), 7.72 (m, 1H), 7.59 (m, 1H), 7.47 (m, 2H), 2.37 (s, 3H). |
| D-4 | 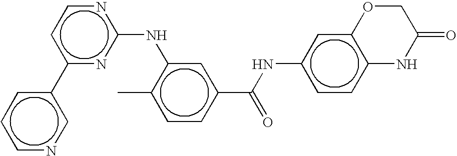 | 453.2 | |
| D-5 | 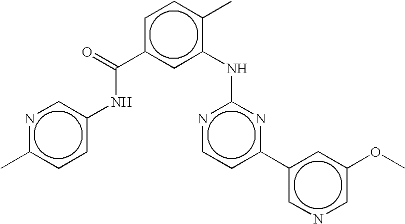 | 427.2 | |
| D-6 | 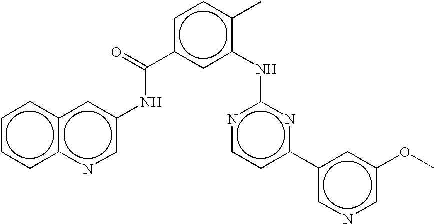 | 463.2 | |
| D-7 | 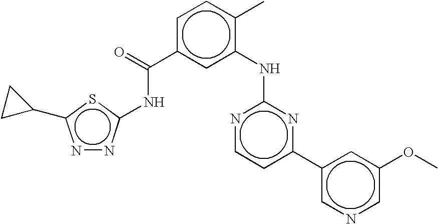 | 460.2 | |
| D-8 | 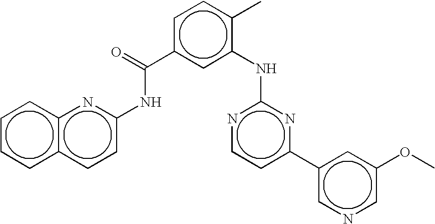 | 463.2 | |
| D-9 | 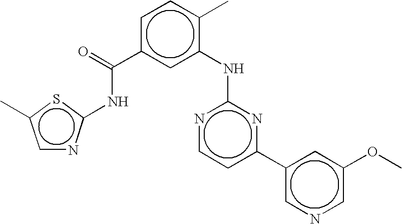 | 433.1 | |
| D-10 | 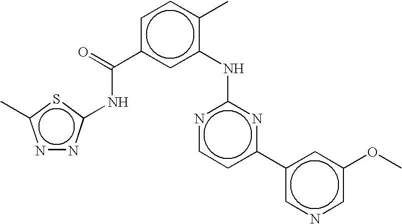 | 434.2 | |
| D-11 | 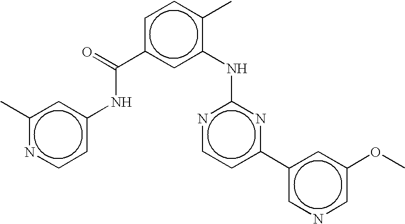 | 427.0 | |
| D-12 | 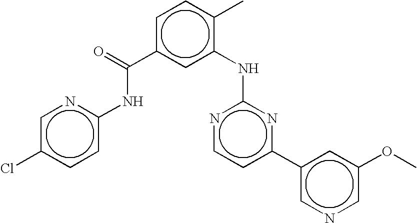 | 447.1 449.1 | |
| E-1 | 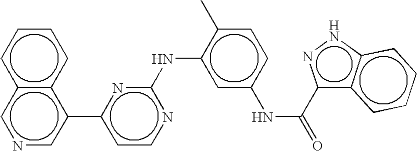 | 472.2 | |
| E-2 | 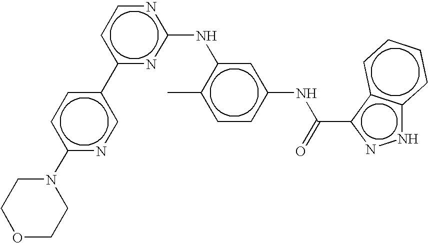 | 507.2 | |
| E-3 | 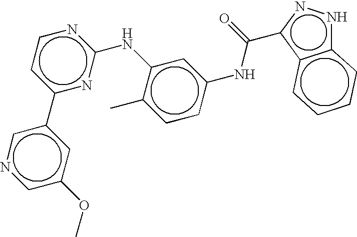 | 452.2 | |
| E-4 | 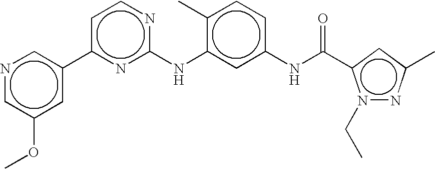 | 444.2 | ¹H NMR (400 MHz, d₄-Methanol) δ 9.12 (s, 1H), 8.64 (s, 1H), 8.59-8.62 (m, 1H), 8.55 (d, J = 5.4 Hz, 1H), 8.23 (s, 1H), 7.54 (d, J = 5.4 Hz, 1H), 7.27-7.35 (m, 2H), 6.70 (s, 1H), 4.45-4.52 (m, 2H), 4.03 (s, 3H), 2.33 (s, 3H), 2.28 (s, 3H), 1.36 (t, J = 7.1 Hz, 3H). |
| E-5 | 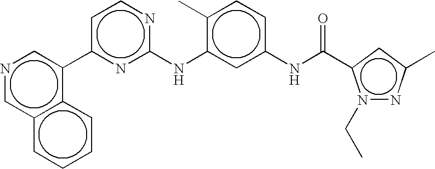 | 464.2 | ¹H NMR (400 MHz, d₄-Methanol) δ 9.61 (s, 1H), 8.78 (s, 1H), 8.58-8.64 (m, 1H), 8.40 (d, J = 8.3 Hz, 1H), 8.15 (s, 1H), 8.03 (t, J = 8.2 Hz, 1H), 7.88 (t, J = 7.6 Hz, 1H), 7.31-7.36 (m, 1H), 7.21-7.27 (m, 2H), 6.66 (s, 1H), 4.42-4.49 (m, 2H), 2.32 (s, 3H), 2.28 (s, 3H), 1.35 (t, J = 7.1 Hz, 3H). |
| E-6 | 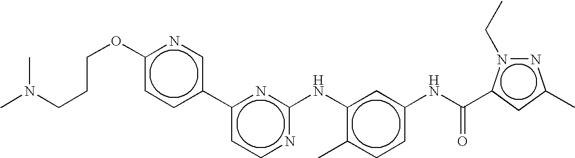 | 515.3 | |
| F1 | 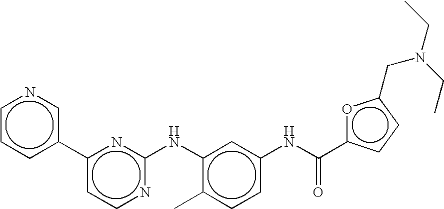 | 457.2 | ¹H NMR (400 MHz, d₆-DMSO) δ 10.14 (s, 1H), 9.28 (s, 1H), 9.01 (s, 1H), 8.70 (d, J = 3.6 Hz, 1H), 8.52 (m, 2H), 7.99 (s, 1H), 7.57 (m, 1H), 7.44 (m, 1H), 7.23 (m, 1H), 6.94 (m, 1H), 4.50 (s, 2H), 3.13 (m, 4H), 2.55 (s, 3H), 1.25 (t, J = 7.2 Hz, 6H). |
| F-2 | 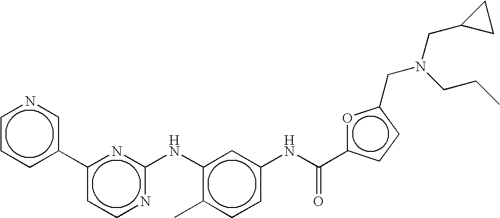 | 497.2 | ¹H NMR (400 MHz, d₆-DMSO) δ 10.14 (s, 1H), 9.28 (s, 1H), 9.05 (s, 1H), 8.71 (dd, J = 4.8, 1.6 Hz, 1H), 8.52 (m, 2H), 8.0 (d, J = 2.0 Hz, 1H), 7.58 (m, 1H), 7.48 (m, 1H), 7.43 (dd, J = 8.4, 2.0 Hz, 1H), 7.23 (d, J = 8.4 Hz, 1H), 6.96 (d, J = 3.6 Hz, 1H), 4.57 (s, 2H), 2.23 (s, 3H), 1.70 (m, 2H), 1.15 (m, 1H), 0.896 (t, J = 7.6 Hz, 3H), 0.66 (m, 2H), 0.41 (m, 2H). |
| F-3 | 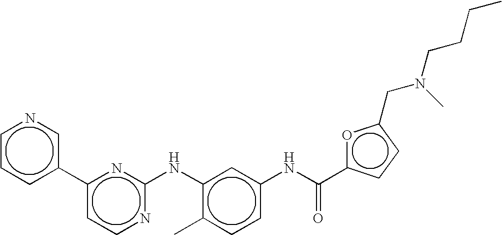 | 471.2 | . ¹H NMR (400 MHz, d₆-DMSO) δ 10.16 (s, 1H), 9.30 (d, J = 1.6 Hz, 1H), 9.05 (s, 1H), 8.72 (dd, J = 4.8 Hz, 1.6 Hz, 1H), 8.53 (d, J = 5.2 Hz, 2H), 8.0 (d, J = 2.0 Hz, 1H), 7.59 (m, 1H), 7.47 (m, 1H), 7.43 (dd, J = 8.0 Hz, 2.4 Hz, 1H), 7.23 (d, J = 8.4 Hz, 1H), 6.93 (d, J = 3.6 Hz, 1H), 4.51 (m, 2H), 3.10 (s, 2H), 2.77 (s, 3H), 2.23 (s, 3H), 1.65 (m, 2H), 1.31 (m, 2H), 0.89 (t, J = 7.2 Hz, 3H). |
| F-4 | 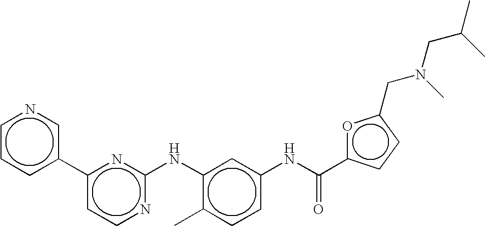 | 471.2 | ¹H NMR (400 MHz, d₆-DMSO) δ 10.2 (s, 1H), 9.29 (d, J = 1.6 Hz, 1H), 9.05 (s, 1H), 8.72 (dd, J = 4.8 Hz, 1.6 Hz, 1H), 8.53 (m, 2H), 8.0 (d, J = 2.0 Hz, 1H), 7.59 (m, 1H), 7.47 (m, 1H), 7.43 (dd, J = 8.0, 2.0 Hz, 1H), 7.23 (d, J = 8.8 Hz, 1H), 6.95 (d, J = 3.2 Hz, 1H), 2.93 (m, 2H), 2.79 (s, 3H), 2.52 (m, 2H), 2.23 (s, 3H), 2.09 (m, 1H), 0.94 (d, J = 6.8 Hz, 6H). |
| F-5 | 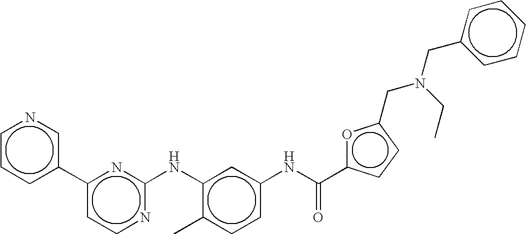 | 519.3 | |
| F-6 | 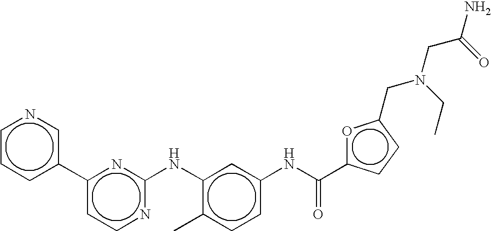 | 486.3 | |
| F-7 | 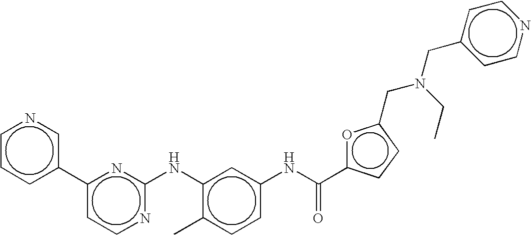 | 520.2 | |
| F-8 | 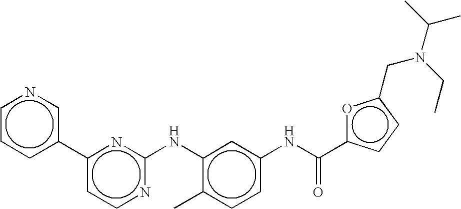 | 471.2 | |
| F-9 | 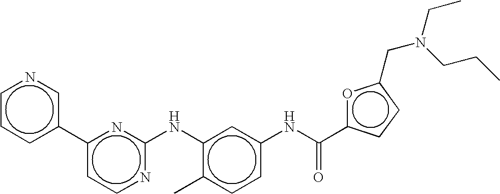 | 471.2 | |
| G-1 | 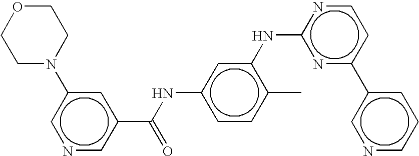 | 468.2 | ¹H NMR (400 MHz, d₆-DMSO) δ 10.5 (s, 1H), 9.35 (s, 1H), 9.07 (s, 1H), 8.76 (d, J = 4.0 Hz, 1H), 8.62 (m, 3H), 8.10 (m, 1H), 8.02 (s, 1H), 7.67 (m, 1H), 7.48 (m, 1H), 7.23 (m, 1H), 3.79 (t, J = 4.8 Hz, 4H), 3.35 (t, J = 4.8 Hz, 4H), 2.25 (s, 3H). |
| G-2 | 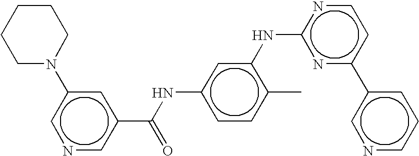 | 466.2 | |
| G-3 | 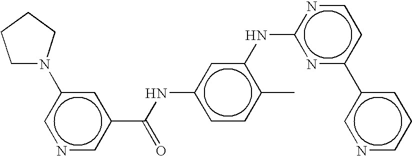 | 452.2 | |
| H-1 | 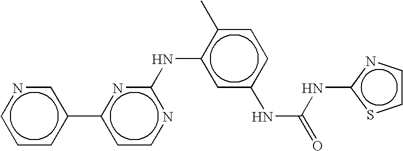 | 404.2 | ¹H NMR (400 MHz, d₆-DMSO) δ 9.29 (s, 1H), 8.95 (s, 2H), 8.73 (s, 1H), 8.60 (d, J = 6.4 Hz, 1H), 8.54 (d, J = 4.8 Hz, 1H), 7.85 (s, 1H), 7.63 (m, 1H), 7.45 (d, J = 5.2 Hz, 1H), 7.36 (d, J = 3.6 Hz, 1H), 7.15 (m, 2H), 7.10 (m, 1H), 2.20 (s, 3H). |
| H-2 | 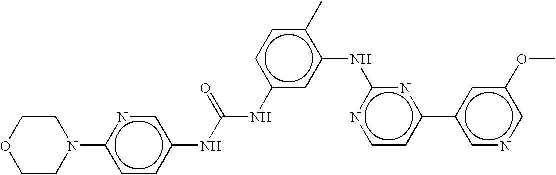 | 513.5 | ¹H NMR (400 MHz, d₆-DMSO) δ 8.95 (s, 1H), 8.89 (s, 1H), 8.81 (s, 1H), 8.67 (m, 1H), 8.52 (d, J = 5.2 Hz, 1H), 8.43 (s, 1H), 8.23 (s, 1H), 8.03 (s, 1H), 7.80 (m, 2H), 7.47 (d, J = 5.2 Hz, 1H), 7.13 (m, 2H), 7.04 (d, J = 8.8 Hz, 1H), 3.87 (m, 2H), 3.73 (m, 2H), 2.55 (m, 4H), 2.51 (s, 3H), 2.19 (s, 3H). |
| H-3 | 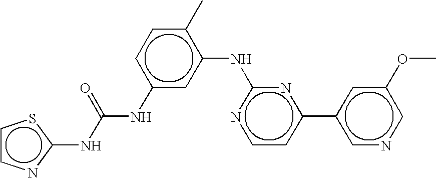 | 434.2 | |
| H-4 | 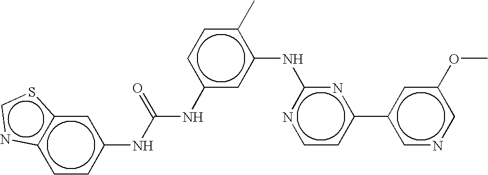 | 484.2 | |
| H-5 | 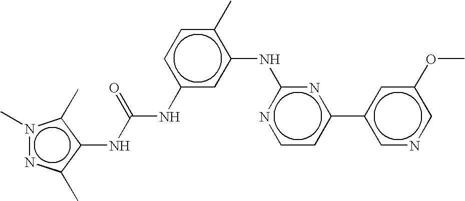 | 459.3 | |
| H-6 | 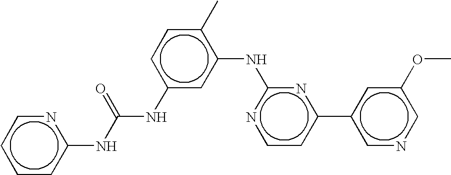 | 428.2 | ¹H NMR (400 MHz, d₆-DMSO) δ 10.46 (s, 1H), 8.98 (s, 1H), 8.91 (s, 1H), 8.54 (d, J = 5 Hz, 1H), 8.44 (d, J = 2.4 Hz, 1H), 8.05 (s, 1H), 7.9 (s, 1H), 7.78 (t, J = 6.8 Hz, 1H), 7.48 (m, 2H), 7.2 (m, 2H), 7.03 (1H, J = 5.7 Hz, 1H), 3.86 (s, 3H), 2.55 (s, 1H), 2.21 (s, 3H). |
| H-7 | 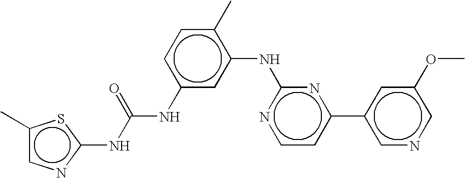 | 448.2 | |
| H-8 | 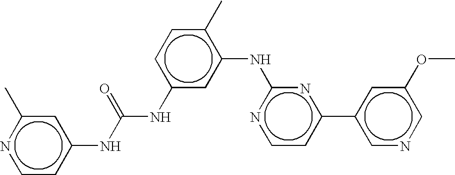 | 442.2 | |
| H-9 | 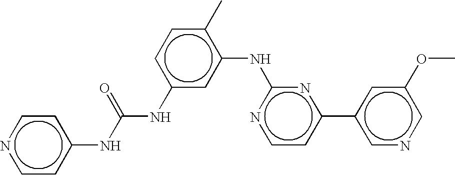 | 428.2 | |
| H-10 | 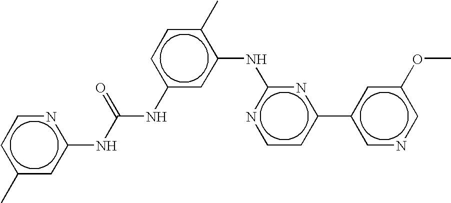 | 442.2 | |
| I-1 | 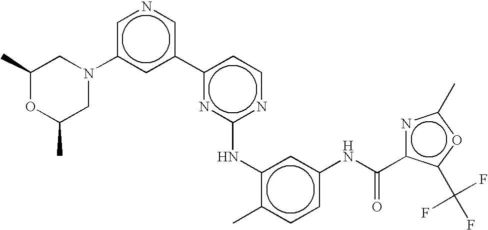 | 568.3 | ¹H NMR (400 MHz, d₆-DMSO) δ 10.52 (s, 1H), 9.04 (s, 1H), 8.7 (dd, J = 5.5, 3.4 Hz, 1H), 8.5 (m, 2H), 8.06 (s, 1H), 8.02 (s, 1H), 7.53 (d, J = 6.8 Hz, 1H), 7.48 (d, J = 3.6 Hz, 1H), 7.21 (d, J = 8.3 Hz, 1H), 3.68 (m, 2H), 2.6 (s, 3H), 2.34 (m, 4H), 2.21 (s, 3H), 1.13 (d, J = 5.3 Hz, 6H). |
| I-2 | 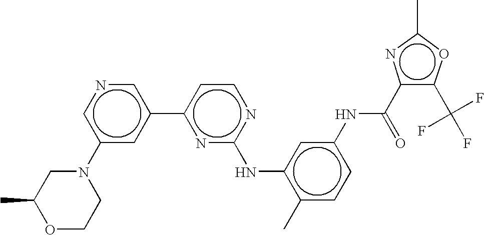 | 554.3 | |
| I-3 | 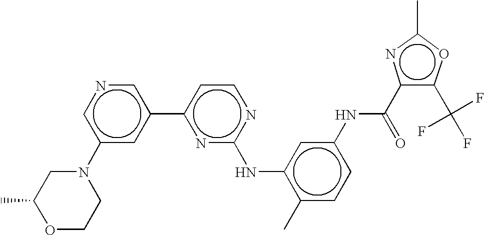 | 554.3 | |

### Example 11

### 3-(2-Methoxy-phenyl)-N-[4-methyl-3-(4-pyridin-3-yl-pyrimidin-2-ylamino)-phenyl]-propionamide

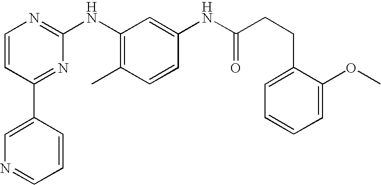

A solution containing approximately 50% of propylphosphonic anhydride in N,N-dimethylformamide (0.77 mL, ˜1.2 mmol) is added in three portions within 20 minutes to a stirred mixture of 4-methyl-N-3-[4-(3-pyridinyl)-2-pyrimidinyl]-1,3-benzenediamine (221.9 mg, 0.8 mmol), 3-(2-methoxy-phenyl)-propionic acid (144.2 mg, 0.8 mmol) and triethylamine (0.887 mL, 6.4 mmol) in 2 mL N,N-dimethylacetamide. After stirring for 24 hours at room temperature, the mixture is treated with a half-saturated aqueous solution of sodium hydrogen carbonate and extracted three times with ethyl acetate. The combined organic extracts are dried (Na₂SO₄) and the solvent is evaporated off under reduced pressure. The crude product is purified by crystallization from acetone to yield the title compound as a brownish solid: MS: 440.2 [M+H]⁺; t_{R} (HPLC, Nucleosil C18; 5-100% CH₃CN+0.1% TFA/H₂O+0.1% TFA for 5 min, flow 1.5 ml/min): 3.91 min; ¹H-NMR (400 MHz, DMSO-d₆, δ):2.16 (s, 3H); 2.55 (t, 2H); 2.84 (t, 2H); 3.78 (s; 3H); 6.83 (t, 1H); 6.93 (d, 1H); 7.09-7.19 (m, 3H); 7.26 (m, 1H); 7.41 (d, 1H); 7.49 (dd, 1H); 7.87 (m, 1H); 8.45 (m, 1H); 8.49 (d, 1H); 8.67 (dd, 1H); 8.91 (s, 1H); 9.24 (m, 1H); 9.80 (s, 1H).

### Example 12

### 1-Ethyl-7-methyl-4-oxo-1,4-dihydro-[1,8-]naphthyridine-3-carboxylic acid [4-methyl-3-(4-pyridin-3-yl-pyrimidin-2-ylamino)-phenyl]-amide

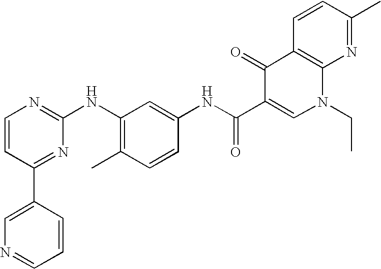

A solution containing approximately 50% of propylphosphonic anhydride in N,N-dimethylformamide (0.77 mL, ˜1.2 mmol) is added in three portions within 20 minutes to a stirred mixture of 4-methyl-N3-[4-(3-pyridinyl)-2-pyrimidinyl]-1,3-benzenediamine (221.9 mg, 0.8 mmol), 1-ethyl-7-methyl-4-oxo-1,4-dihydro-[1,8]naphthyridine-3-carboxylic acid (185.8 mg, 0.8 mmol) and triethylamine (0.887 mL, 6.4 mmol) in 2 mL N,N-dimethylacetamide. After stirring for 24 hours at room temperature, the mixture is distributed between a half-saturated aqueous solution of sodium hydrogen carbonate and ethyl acetate. The precipitate is filtered off, washed with H₂O, methanol and diethyl ether and dried in vacuo to yield the title compound as a brownish solid: MS: 492.1 [M+H]⁺; t_{R} (HPLC, Nucleosil C18; 5-100% CH₃CN+0.1% TFA/H₂O+0.1% TFA for 5 min, flow 1.5 ml/min): 4.23 min; ¹H-NMR (400 MHz, DMSO-d₆, 6): 1.41 (t, 3H); 2.22 (s, 3H); 2.67 (s, 3H); 4.61 (q, 2H); 7.21 (d, 1H); 7.41 (m, 1H); 7.45 (d, 1H); 7.50-7.58 (m, 2H); 8.07 (d, 1H); 8.47-8.55 (m, 2H); 8.63 (d, 1H); 8.68 (dd, 1H); 8.96 (s, 1H); 9.10 (s, 1H); 9.28 (m, 11H); 12.19 (s, 1H).

### Example 13

### 1-Methyl-1H-indole-2-carboxylic acid [4-methyl-3-(4-pyridin-3-yl-pyrimidin-2-ylamino)-phenyl]-amide

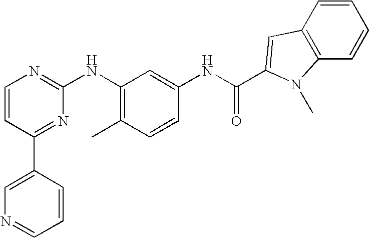

The title compound is prepared analogously as described in Example 11 using 1-methyl-1H-indole-2-carboxylic acid instead of 3-(2-methoxy-phenyl)-propionic acid:Brownish solid; MS: 435.1 [M+H]⁺; t_{R} (HPLC, Nucleosil C18; 5-100% CH₃CN+0.1% TFA/H₂O+0.1% TFA for 5 min, flow 1.5 ml/min): 4.15 min; ¹H-NMR (400 MHz, DMSO-d₆, 6): 2.22 (s, 3H); 4.00 (s, 3H); 7.11 (t, 1H); 7.20 (d, 1H); 7.29 (m, 2H); 7.41-7.58 (m, 4H); 7.68 (d, 1H); 8.06 (d, 1H); 8.14 (dd, 1H); 8.46-8.52 (m, 2H); 8.68 (dd, 1H); 8.99 (s, 1H); 9.30 (m, 1H); 10.28 (s, 1H).

### Example 14

### 5-Nitro-furan-2-carboxylic acid [4-methyl-3-(4-pyridin-3-yl-pyrimidin-2-ylamino)-phenyl]-amide

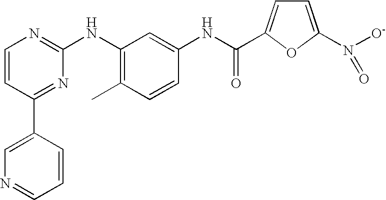

The title compound is prepared analogously as described in Example 11 using 5-nitro-furan-2-carboxylic acid instead of 3-(2-methoxy-phenyl)-propionic acid: Brownish solid; MS: 417.1 [M+H]⁺; t_{R} (HPLC, Nucleosil C18; 5-100% CH₃CN+0.1% TFA/H₂O+0.1% TFA for 5 min, flow 1.5 ml/min): 3.65 min; ¹H-NMR (400 MHz, DMSO-d₆, δ): 2.22 (s, 3H); 7.22 (d, 1H); 7.41-7.54 (m, 3H); 7.63 (d, 1H); 7.80 (d, 1H); 8.02 (m, 1H); 8.44 (dt, 1H); 8.51 (d, 1H); 8.67 (dd, 1H); 9.02 (s, 1H); 9.25 (d, 1H); 10.59 (s, 1H).

### Example 15

### {2-[4-Methyl-3-(4-pyridin-3-ylamidin-2-ylamino)-benzoylamino]-thiazol-4-yl}-acetic Acid Ethyl Ester

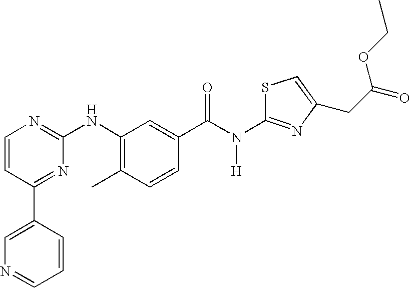

A solution containing approximately 50% of propylphosphonic anhydride in N,N-dimethylformamide (0.674 mL, ˜1.05 mmol) is added in three portions within 20 minutes to a stirred mixture of 4-methyl-3-(4-pyridin-3-yl-pyrimidin-2-ylamino)-benzoic acid (214.4 mg, 0.7 mmol), (2-amino-thiazol-4-yl)-acetic acid ethyl ester (130.4 mg, 0.7 mmol) and triethylamine (0.776 mL, 5.6 mmol) in 2 mL N,N-dimethylformamide. After stirring for 24 hours at room temperature, the mixture is distributed between a half-saturated aqueous solution of sodium hydrogen carbonate and ethyl acetate. The precipitate is filtered off, washed with H₂O and ethyl acetate and dried in vacuo to yield the title compound as a beige solid: MS: 475.1 [M+H]⁺; ¹ H-NMR (400 MHz, DMSO-d₆, δ): 1.16 (t, 3H); 2.32 (s, 3H); 3.71 (s, 2H); 4.06 (q, 2H); 7.02 (s, 1H); 7.38 (d, 1H); 7.47-7.55 (m, 2H); 7.85 (dd, 1H); 8.38-8.46 (m, 2H); 8.54 (m, 1H); 8.68 (dd, 1H); 9.11 (s, 1H); 9.26 (m, 1H); 12.58 (br. s, 1H).

### Example 16

### 5-Methyl-2-phenyl-2H-[1,2,3]triazole-4-carboxylic acid [4-methyl-3-(4-pyridin-3-yl-pyrimidin-2-ylamino)-phenyl]-amide

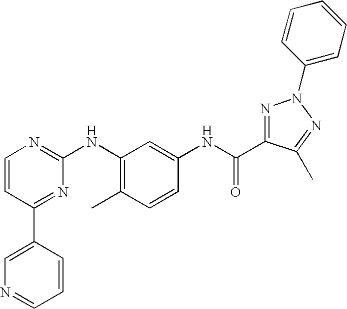

A solution containing approximately 50% of propylphosphonic anhydride in N,N-dimethylformamide (0.70 mL, ˜1.08 mmol) is added in three portions within 20 minutes to a stirred mixture of 4-methyl-N3-[4-(3-pyridinyl)-2-pyrimidinyl]-1,3-benzenediamine (200 mg, 0.72 mmol), 5-methyl-2-phenyl-2H-[1,2,3]triazole-4-carboxylic acid (146.3 mg, 0.72 mmol) and triethylamine (0.798 mL, 5.76 mmol) in 2 mL N,N-dimethylformamide. After stirring for 72 hours at room temperature, the solvent is removed in vacuo and the residue is distributed between a half-saturated aqueous solution of sodium hydrogen carbonate and ethyl acetate. The precipitate is filtered off, washed with H₂O and ethyl acetate and dried in vacuo to afford the title compound as a beige solid: MS: 463.1 [M+H]⁺; t_{R} (HPLC, Nucleosil C18; 5-100% CH₃CN+0.1% TFA/H₂O+0.1% TFA for 5 min, flow 1.5 mL/min): 4.49 min; ¹H-NMR (400 MHz, DMSO-d₆, 6): 2.23 (s, 3H); 2.57 (s, 3H); 7.22 (d, 1H); 7.41-7.63 (m, 6H); 8.12 (m, 2H); 8.17 (m, 1H); 8.46-8.54 (m, 2H); 8.68 (dd, 1H); 8.98 (s, 1H); 9.27 (d, 1H); 10.32 (s, 1H).

### Example 17

### 6-Hydroxy-N-[4-methyl-3-(4-pyridin-3-yl-pyrimidin-2-ylamino)-phenyl]-nicotinamide

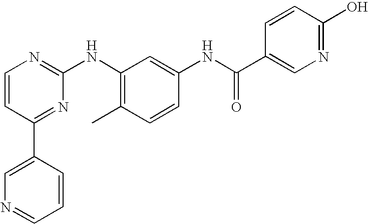

The title compound is prepared analogously as described in Example 16 using 6-hydroxy-nicotinic acid instead of 5-methyl-2-phenyl-2H-[1,2,3]triazole-4-carboxylic acid. The filtered precipitate is washed with H₂O, methanol, CH₂Cl₂ and diethyl ether and dried in vacuo to yield the title compound as a beige powder: MS: 399.2 [M+H]⁺; t_{R} (HPLC, Nucleosil C18; 5-100% CH₃CN+0.1% TFA/H₂O+0.1% TFA for 5 min, flow 1.5 ml/min): 2.99 min; ¹H-NMR (400 MHz, DMSO-d₆, δ): 2.21 (s, 3H); 6.40 (d, 1H); 7.19 (d, 1H); 7.37-7.54 (m, 3H); 7.93-8.02 (m, 2H); 8.18 (m, 1H); 8.43-8.53 (m, 2H); 8.68 (dd, 1H); 8.90 (s, 1H); 9.27 (d, 1H); 9.90 (s, 1H); (12.02 (br. s, 1H).

### Example 18

### 2-Hydroxy-N-[4-methyl-3-(4-pyridin-3-ylamidin-2-ylamino)-phenyl]-nicotinamide

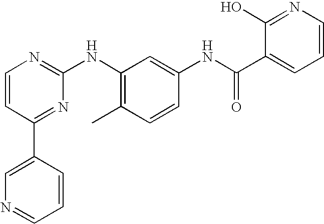

The title compound is prepared analogously as described in Example 16 using 2-hydroxy-nicotinic acid instead of 5-methyl-2-phenyl-2H-[1,2,3]triazole-4-carboxylic acid: Brownish solid; MS: 399.2 [M+H]⁺; t_{R} (HPLC, Nucleosil C18; 5-100% CH₃CN+0.1% TFA/H₂O+0.1% TFA for 5 min, flow 1.5 ml/min): 3.29 min; ¹H-NMR (400 MHz, DMSO-d₆, δ): 2.22 (s, 3H); 6.57 (m, 1H); 7.19 (d, 1H); 7.30-7.60 (m, 3H); 7.77 (m, 1H); 8.07 (m, 1H); 8.39-8.55 (m, 3H); 8.67 (m, 1H); 8.92 (s, 1H); 9.26 (m, 1H); 12.17 (s, 1H); 12.72 (br. S, 1H).

### Example 19

### 3-Hydroxy-pyridine-2-carboxylic acid [4-methyl-3-(4-pyridin-3-yl-pyrimidin-2-ylamino)-phenyl]-amide

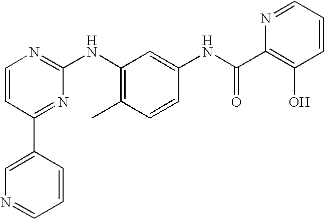

The title compound is prepared analogously as described in Example 16 using 3-hydroxy-pyridine-2-carboxylic acid instead of 5-methyl-2-phenyl-2H-[1,2,3]triazole-4-carboxylic acid. The ethyl acetate layer is diluted with CH₂Cl₂/methanol (9:1), dried over Na₂SO₄ and evaporated in vacuo. The residue thus obtained is crystallized with methanol to yield the title compounds as a beige solid: MS: 399.2 [M+H]⁺; t_{R} (HPLC, Nucleosil C18; 5-100% CH₃CN+0.1% TFA/H₂O+0.1% TFA for 5 min, flow 1.5 mL/min): 3.89 min; ¹H-NMR (400 MHz, DMSO-d₆, 6): 2.24 (s, 3H); 7.23 (d, 1H); 7.41-7.61 (m, 5H); 8.25 (m, 2H); 8.45-8.55 (m, 2H); 8.68 (dd, 1H); 8.97 (s, 1H); 9.31 (d, 1H); 10.82 (s, 1H); 12.17 (s, 1H).

### Example 20

### 2-Methyl-N-[4-methyl-3-(4-pyridin-3-yl-pyrimidin-2-ylamino)-phenyl]-nicotinamide

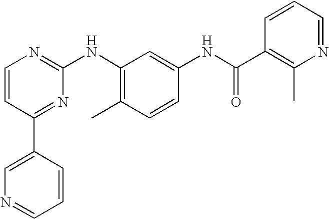

A solution containing approximately 50% of propylphosphonic anhydride in N,N-dimethylformamide (0.77 mL, ˜1.2 mmol) is added in three portions within 20 minutes to a stirred mixture of 4-methyl-N3-[4-(3-pyridinyl)-2-pyrimidinyl]-1,3-benzenediamine (221.9 mg, 0.8 mmol), 2-methyl-nicotinic acid (109.7 mg, 0.8 mmol) and triethylamine (0.887 mL, 6.4 mmol) in 2 mL N,N-dimethylformamide. After stirring for 24 hours at room temperature, the mixture is treated with a half-saturated aqueous solution of sodium hydrogen carbonate and extracted three times with ethyl acetate. The combined organic extracts are dried (Na₂SO₄) and the solvent is evaporated off under reduced pressure. The crude product is purified by crystallization from CH₂Cl₂/diethyl ether to yield the title compound as a brownish solid: MS: 397.2 [M+H]⁺; t_{R} (HPLC, Nucleosil C18; 5-100% CH₃CN+0.1% TFA/H20+0.1% TFA for 5 min, then 100% CH₃CN+0.1% TFA for 2 min, flow 1.5 ml/min): 2.91 min; ¹H-NMR (400 MHz, DMSO-d₆, δ): 2.21 (s, 3H); 2.57 (s, 3H); (q, 4H); 7.20 (d, 1H); 7.30-7.54 (m, 4H); 7.84 (m, 1H); 8.06 (m, 1H); 8.42-8.57 (m, 3H); 8.68 (dd, 1H); 9.00 (s, 1H); 9.26 (d, 1H); 10.40 (s, 1H).

### Assays

Compounds of the present invention are assayed to measure their capacity to selectively inhibit the proliferation of wild type Ba/F3 cells and Ba/F3 cells transformed with Tel c-kit kinase and Tel PDGFR fused tyrosine kinases. In addition, compounds of the invention selectively inhibit SCF dependent proliferation in Mo7e cells. Further, compounds are assayed to measure their capacity to inhibit Abl, ARG, BCR-Abl, BRK, EphB, Fms, Fyn, KDR, c-Kit, LCK, PDGF-R, b-Raf, c-Raf, SAPK2, Src, Tie2 and TrkB kinases.

### Ba/F3 FL FLT3 Proliferation Assay

The murine cell line used is the Ba/F3 murine pro-B cell line that overexpresses full length FLT3 construct. These cells are maintained in RPMI 1640/10% fetal bovine serum (RPMI/FBS) supplemented with penicillin 50 μg/mL, streptomycin 50 μg/mL and L-glutamine 200 mM with the addition of murine recombinant IL3. Ba/F3 full length FLT3 cells undergo IL3 starvation for 16 hours and then plated into 384 well TC plates at 5,000 cells in 25 uL media per well and test compound at 0.06 nM to 10 μM is added. After the compound addition FLT3 ligand or IL3 for cytotoxicity control are added in 25 ul media per well at the appropriate concentrations. The cells are then incubated for 48 hours at 37° C., 5% CO₂. After incubating the cells, 25 μL of BRIGHT GLO® (Promega) is added to each well following manufacturer's instructions and the plates are read using Analyst GT—Luminescence mode—50000 integration time in RLU.

### Human TG-HA-VSMC Proliferation Assay

Human TG-HA-VSMC cells (ATCC) are grown in DMEM supplemented with 10% FBS to 80-90% confluence prior to resuspending in DMEM supplemented with 1% FBS and 30 ng/mL recombinant human PDGF-BB at 6e4 cells/mL. Cells are then aliquoted into 384 well plates at 50uL/well, incubated for 20 h at 37° C., then treated with 0.5 uL of 100× compounds for 48 h at 37° C. After the treatment, 25 uL of CellTiter-Glo is added to each well for 15 min, then the plates are read on the CLIPR (Molecular Devices)

### Proliferation Assay BaF3 Library—Bright glo Readout Protocol

Compounds are tested for their ability to inhibit the proliferation of wt Ba/F3 cells and Ba/F3 cells transformed with Tel fused tyrosine kinases. Untransformed Ba/F3 cells are maintained in media containing recombinant IL3. Cells are plated into 384 well TC plates at 5,000 cells in 50 ul media per well and test compound at 0.06 nM to 10 μM is added. The cells are then incubated for 48 hours at 37° C., 5% CO₂. After incubating the cells, 25 μL of BRIGHT GLO® (Promega) is added to each well following manufacturer's instructions and the plates are read using Analyst GT—Luminescence mode—50000 integration time in RLU. IC₅₀ values, the concentration of compound required for 50% inhibition, are determined from a dose response curve.

### Mo7e Assay

The compounds described herein are tested for inhibition of SCF dependent proliferation using Mo7e cells which endogenously express c-kit in a 96 well format. Briefly, two-fold serially diluted test compounds (Cmax=10 μM) are evaluated for their antiproliferative activity of Mo7e cells stimulated with human recombinant SCF. After 48 hours of incubation at 37° C., cell viability is measured by using a MTT colorimetric assay from Promega.

### Inhibition of Cellular BCR-Abl Dependent Proliferation (High Throughput Method)

The murine cell line used is the 32D hemopoietic progenitor cell line transformed with BCR-Abl cDNA (32D-p210). These cells are maintained in RPMI/10% fetal calf serum (RPMI/FCS) supplemented with penicillin 50 μg/mL, streptomycin 50 μg/mL and L-glutamine 200 mM. Untransformed 32D cells are similarly maintained with the addition of 15% of WEHI conditioned medium as a source of IL3.

50 μL of a 32D or 32D-p210 cells suspension are plated in Greiner 384 well microplates (black) at a density of 5000 cells per well. 50mL of test compound (1 mM in DMSO stock solution) is added to each well (STI571 is included as a positive control). The cells are incubated for 72 hours at 37° C., 5% CO₂. 10 μL of a 60% Alamar Blue solution (Tek diagnostics) is added to each well and the cells are incubated for an additional 24 hours. The fluorescence intensity (Excitation at 530 nm, Emission at 580 nm) is quantified using the Acquest™ system (Molecular Devices).

### Inhibition of Cellular BCR-Abl Dependent Proliferation

32D-p210 cells are plated into 96 well TC plates at a density of 15,000 cells per well. 50 μL of two fold serial dilutions of the test compound (Cₘₐₓ is 40 μM) are added to each well (STI 571 is included as a positive control). After incubating the cells for 48 hours at 37° C., 5% CO₂, 15 μL of MTT (Promega) is added to each well and the cells are incubated for an additional 5 hours. The optical density at 570 nm is quantified spectrophotometrically and IC₅₀ values, the concentration of compound required for 50% inhibition, determined from a dose response curve.

### Effect on Cell Cycle Distribution

32D and 32D-p210 cells are plated into 6 well TC plates at 2.5×10⁶ cells per well in 5 mL of medium and test compound at 1 or 10 μM is added (STI571 is included as a control). The cells are then incubated for 24 or 48 hours at 37° C., 5% CO₂. 2 mL of cell suspension is washed with PBS, fixed in 70% EtOH for 1 hour and treated with PBS/EDTA/RNase A for 30 minutes. Propidium iodide (Cf=10 μg/ml) is added and the fluorescence intensity is quantified by flow cytometry on the FACScalibur™ system (BD Biosciences). Test compounds of the present invention demonstrate an apoptotic effect on the 32D-p210 cells but do not induce apoptosis in the 32D parental cells.

### Effect on Cellular BCR-Abl Autophosphorylation

BCR-Abl autophosphorylation is quantified with capture Elisa using a c-abl specific capture antibody and an antiphosphotyrosine antibody. 32D-p210 cells are plated in 96 well TC plates at 2×10⁵ cells per well in 50 μL of medium. 50 μL of two fold serial dilutions of test compounds (Cₘₐₓ is 10 μM) are added to each well (STI571 is included as a positive control). The cells are incubated for 90 minutes at 37° C., 5% CO₂. The cells are then treated for 1 hour on ice with 150 μL of lysis buffer (50 mM Tris-HCl, pH 7.4, 150 mM NaCl, 5 mM EDTA, 1 mM EGTA and 1% NP-40) containing protease and phosphatase inhibitors. 50 μL of cell lysate is added to 96 well optiplates previously coated with anti-abl specific antibody and blocked. The plates are incubated for 4 hours at 4° C. After washing with TBS-Tween 20 buffer, 50 μL of alkaline-phosphatase conjugated anti-phosphotyrosine antibody is added and the plate is further incubated overnight at 4° C. After washing with TBS-Tween 20 buffer, 90 μL of a luminescent substrate are added and the luminescence is quantified using the Acquest™ system (Molecular Devices). Test compounds of the invention that inhibit the proliferation of the BCR-Abl expressing cells, inhibit the cellular BCR-Abl autophosphorylation in a dose-dependent manner.

### Effect on Proliferation of Cells Expressing Mutant Forms of Bcr-abl

Compounds of the invention are tested for their antiproliferative effect on Ba/F3 cells expressing either wild type or the mutant forms of BCR-Abl (G250E, E255V, T3151, F317L, M351T) that confers resistance or diminished sensitivity to STI571. The antiproliferative effect of these compounds on the mutant-BCR-Abl expressing cells and on the non transformed cells were tested at 10, 3.3, 1.1 and 0.37 μM as described above (in media lacking IL3). The IC₅₀ values of the compounds lacking toxicity on the untransformed cells were determined from the dose response curves obtained as describe above.

### FGFR3 (Enzymatic Assay)

Kinase activity assay with purified FGFR3 (Upstate) is carried out in a final volume of 10 μL containing 0.25 μg/mL of enzyme in kinase buffer (30 mM Tris-HCl pH7.5, 15 mM MgCl₂, 4.5 mM MnCl₂, 15 μM Na₃VO₄ and 50 μg/mL BSA), and substrates (5 μg/mLbiotin-poly-EY(Glu, Tyr) (CIS-US, Inc.) and 3 μM ATP). Two solutions are made: the first solution of 5 μL contains the FGFR3 enzyme in kinase buffer was first dispensed into 384-format ProxiPlate® (Perkin-Elmer) followed by adding 50 mL of compounds dissolved in DMSO, then 5 μL of second solution contains the substrate (poly-EY) and ATP in kinase buffer was added to each wells. The reactions are incubated at room temperature for one hour, stopped by adding 10 μL of HTRF detection mixture, which contains 30 mM Tris-HCl pH7.5, 0.5 M KF, 50 mM ETDA, 0.2 mg/mL BSA, 15 μg/mL streptavidin-XL665 (CIS-US, Inc.) and 150 ng/mL cryptate conjugated anti-phosphotyrosine antibody (CIS-US, Inc.). After one hour of room temperature incubation to allow for streptavidin-biotin interaction, time resolved florescent signals are read on Analyst GT (Molecular Devices Corp.). IC₅₀ values are calculated by linear regression analysis of the percentage inhibition of each compound at 12 concentrations (1:3 dilution from 50 μM to 0.28 nM). In this assay, compounds of the invention have an IC₅₀ in the range of 10 nM to 2 μM.

### FGFR3 (Cellular Assay)

Compounds of the invention are tested for their ability to inhibit transformed Ba/F3-TEL-FGFR3 cells proliferation, which is depended on FGFR3 cellular kinase activity. Ba/F3-TEL-FGFR3 are cultured up to 800,000 cells/mL in suspension, with RPMI 1640 supplemented with 10% fetal bovine serum as the culture medium. Cells are dispensed into 384-well format plate at 5000 cell/well in 50 μL culture medium. Compounds of the invention are dissolved and diluted in dimethylsufoxide (DMSO). Twelve points 1:3 serial dilutions are made into DMSO to create concentrations gradient ranging typically from 10 mM to 0.05 μM. Cells are added with 50 mL of diluted compounds and incubated for 48 hours in cell culture incubator. AlamarBlue® (TREK Diagnostic Systems), which can be used to monitor the reducing environment created by proliferating cells, are added to cells at final concentration of 10%. After an additional four hours of incubation in a 37° C. cell culture incubator, fluorescence signals from reduced AlamarBlue® (Excitation at 530 nm, Emission at 580 nm) are quantified on Analyst GT (Molecular Devices Corp.). IC₅₀ values are calculated by linear regression analysis of the percentage inhibition of each compound at 12 concentrations.

### FLT3 and PDGFRβ (Cellular Assay)

The effects of compounds of the invention on the cellular activity of FLT3 and PDGFRβ are conducted using identical methods as described above for FGFR3 cellular activity, except that instead of using Ba/F3-TEL-FGFR3, Ba/F3-FLT3-ITD and Ba/F3-Tel-PDGFR, are used, respectively.

b-Raf—Enzymatic Assay

Compounds of the invention are tested for their ability to inhibit the activity of b-Raf. The assay is carried out in 384-well MaxiSorp plates (NUNC) with black walls and clear bottom. The substrate, IκBα is diluted in DPBS (1:750) and 15 μL is added to each well. The plates are incubated at 4° C. overnight and washed 3 times with TBST (25 mM Tris, pH 8.0, 150 mM NaCl and 0.05% Tween-20) using the EMBLA plate washer. Plates are blocked by Superblock (15 μL/well) for 3 hours at room temperature, washed 3 times with TBST and pat-dried. Assay buffer containing 20 μM ATP (10 μL) is added to each well followed by 100 nL or 500nL of compound. B-Raf is diluted in the assay buffer (1 μL into 25 μL) and 10 μL of diluted b-Raf is added to each well (0.4 μg/well). The plates are incubated at room temperature for 2.5 hours. The kinase reaction is stopped by washing the plates 6 times with TBST. Phosph-IκBα (Ser32/36) antibody is diluted in Superblock (1:10,000) and 15 μL is added to each well. The plates are incubated at 4° C. overnight and washed 6 times with TBST. AP-conjugated goat-anti-mouse IgG is diluted in Superblock (1:1,500) and 15 μL is added to each well. Plates are incubated at room temperature for 1 hour and washed 6 times with TBST. 15 μL of fluorescent Attophos AP substrate (Promega) is added to each well and plates are incubated at room temperature for 15 minutes. Plates are read on Acquest or Analyst GT using a Fluorescence Intensity Program (Excitation 455 nm, Emission 580 nm).

b-Raf—Cellular Assay

Compounds of the invention are tested in A375 cells for their ability to inhibit phosphorylation of MEK. A375 cell line (ATCC) is derived from a human melanoma patient and it has a V599E mutation on the B-Raf gene. The levels of phosphorylated MEK are elevated due to the mutation of B-Raf. Sub-confluent to confluent A375 cells are incubated with compounds for 2 hours at 37° C. in serum free medium. Cells are then washed once with cold PBS and lysed with the lysis buffer containing 1% Triton X100. After centrifugation, the supernatants are subjected to SDS-PAGE, and then transferred to nitrocellulose membranes. The membranes are then subjected to western blotting with anti-phospho-MEK antibody (ser217/221) (Cell Signaling). The amount of phosphorylated MEK is monitored by the density of phospho-MEK bands on the nitrocellulose membranes.

### Upstate KinaseProfiler™—Radio-Enzymatic Filter Binding Assay

Compounds of the invention are assessed for their ability to inhibit individual members of the kinase panel. The compounds are tested in duplicates at a final concentration of 10 μM following this generic protocol. Note that the kinase buffer composition and the substrates vary for the different kinases included in the “Upstate KinaseProfiler™” panel. Kinase buffer (2.5 μL, 10×—containing MnCl₂ when required), active kinase (0.001-0.01 Units; 2.5 μL), specific or Poly(Glu-4-Tyr) peptide (5-500 μM or 0.01 mg/ml) in kinase buffer and kinase buffer (50 μM; 5 μL) are mixed in an eppendorf on ice. A Mg/ATP mix (10 L; 67.5 (or 33.75) mM MgCl₂, 450 (or 225) μM ATP and 1 μCi/μl [γ-³²P]-ATP (3000 Ci/mmol)) is added and the reaction is incubated at about 30° C. for about 10 minutes. The reaction mixture is spotted (20 μL) onto a 2 cm×2 cm P81 (phosphocellulose, for positively charged peptide substrates) or Whatman No. 1 (for Poly (Glu-4-Tyr) peptide substrate) paper square. The assay squares are washed 4 times, for 5 minutes each, with 0.75% phosphoric acid and washed once with acetone for 5 minutes. The assay squares are transferred to a scintillation vial, 5 ml scintillation cocktail are added and ³²P incorporation (cpm) to the peptide substrate is quantified with a Beckman scintillation counter. Percentage inhibition is calculated for each reaction.

Antimalarial Assay using SYBR Green I

Compounds of the present invention can be assayed to measure their capacity to inhibit the proliferation of parasitemia in infected red blood cells. The proliferation is quantified by addition of SYBR Green I (Invitrogen)® dye which has a high affinity for double stranded DNA.

For drug screening, 20 μL of screening media, containing no human serum, is dispensed into 3 assay plates. 50nL of each of the compounds of the invention, including antimalarial controls (chloroquine and artimesinin), are then transferred into the assay plates. 50nL of DMSO is transferred into the baseline and background control plates. Then 30 μL of a suspension of P. falciparum infected human red blood cells in screening media is dispensed into the assay plates and the baseline control plate such that the final hematocrit is 2.5% with a final parasitemia of 3%. Non-infected red blood cells are dispensed into the background control plate such that the final hematocrit is 2.5%. The plates are placed in a 37° C. incubator for 72 hours with a 93% N₂, 4% CO₂, and 3% O₂ gas mixture. 10 μL of a 10× solution of SYBR Green 10 is dispensed into the plates. The plates are sealed and placed in a −80° C. freezer overnight for the lysis of the red blood cells. The plates are thawed and left at room temperature overnight for optimal staining. The fluorescence intensity is measured (excitation 497 nm, emission 520 nm) using the Acquest system (Molecular Devices). The percentage inhibition is calculated for each compound.

Compounds of Formula I, in free form or in pharmaceutically acceptable salt form, exhibit valuable pharmacological properties, for example, as indicated by the in vitro tests described in this application.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

We claim:

1. A compound of Formula I: 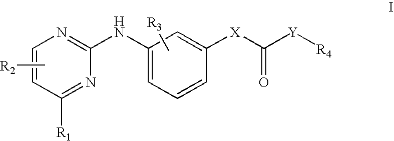
in which
X is selected from a bond and NH;
Y is selected from a bond and NH;
R₁ is selected from cyclohexyl, pyridinyl, quinolinyl, isoquinolinyl and phenyl; wherein said cyclohexyl, pyridinyl, quinolinyl, isoquinolinyl or phenyl of R₁ can be optionally substituted with 1 to 3 radicals independently selected from halo, C₁₋₄alkyl, C₁₋₆alkoxy, halo-substituted-C₁₋₆alkyl, halo-substituted-C₁₋₆alkoxy, —NR₅ₐR₅b, —OX₁NR₅ₐR_{5b} and heterocyclyl; wherein X₁ is independently selected from a bond and C₁₋₄alkylene; and R₅ₐ and R_{5b} are independently selected from hydrogen, C₁₋₆alkyl, C₁₋₆alkoxy, halo-substituted-C₁₋₆alkyl and halo-substituted-C₁₋₆alkoxy; is selected from halo, C₁₋₆alkyl, C₁₋₆alkoxy, halo-substituted-C₁₋₆alkyl and halo-substituted-C₁₋₆alkoxy;
R₂ is selected from halo, C₁₋₆alkyl, C₁₋₆alkoxy, halo-substituted-C₁₋₆alkyl and halo-substituted-C₁₋₆alkoxy;
R₃ is heteroaryl substituted with 1 to 3 radicals independently selected from halo, cyano, C₁₋₆alkyl, C₁₋₆alkoxy, halo-substituted-C₁₋₆alkyl, halo-substituted-C₁₋₆alkoxy, C₆₋₁₀aryl-C₀₋₄alkyl, heteroaryl, heterocyclyl, —X₁NR₅R₅X₁NR₅OR₅, —X₁NR₅X₁OR₅, —X₁NR₅X₁C(O)NR₅R₅, —X₁S(O)₂NR₅R₅, —X₁S(O)₂R₅, —X₁NR₅R₅, —X₁NR₅OR₅, —X₁C(O)R₅, —X₁OX₂OR₅, —OX₁R₅, —X₁R₅, —X₁C(O)OR₅, —X₁OR₅ and —X₁₀X₁OR₅; wherein each X₁ is independently selected from a bond and C₁₋₄alkylene; X₂ is C₁₋₄alkylene; and each R₅ is independently selected from hydrogen, C₁₋₆alkyl, C₂₋₆alkenyl, C₃₋₁₂cycloalkyl, C₆₋₁₀aryl-C₀₋₄alkyl, heteroaryl-C₀₋₄alkyl and heterocyclyl;
wherein said aryl, cycloalkyl, heteroaryl or heterocyclyl substituents of R₄ can optionally be further substituted with 1 to 3 radicals independently selected from halo, hydroxy, cyano, C₁₋₆alkyl, C₁₋₆alkoxy, halo-substituted-C₁₋₆alkyl, halo-substituted-C₁₋₆alkoxy, -L-OR₆, -L-C(O)OR₆, -L-C(O)NR₆R₆ and -L-R₆; wherein L is selected from a bond and C₁₋₄alkylene; and R₆ is selected from hydrogen, C₁₋₆alkyl and heterocyclyl; with the proviso that R₄ is not pyridin-3-yl substituted by a trifluoromethyl radical; and the pharmaceutically acceptable salts thereof.

2. The compound of claim 1 of Formula Ia: 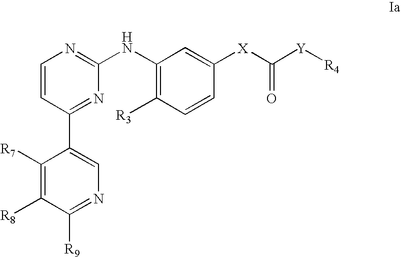
in which:
X is selected from a bond and NH;
Y is selected from a bond and NH; wherein either X or Y, but not both, is a bond;
R₃ is selected from halo, methyl, methoxy, trifluoromethyl and trifluoromethoxy;
R₄ is heteroaryl substituted with 1 to 3 radicals independently selected from halo, cyano, C₁₋₆alkyl, C₁₋₆alkoxy, halo-substituted-C₁₋₆alkyl, halo-substituted-C₁₋₆alkoxy, C₆₋₁₀aryl-C₀₋₄alkyl, heteroaryl, heterocyclyl, —X₁NR₅R₅, —X₁NR₅OR₅, —X₁NR₅X₁OR₅, —X₁NR₅X₁C(O)NR₅R₅, —X₁S(O)₂NR₅R₅, —X₁S(O)₂R₅, —X₁NR₅R₅, —X₁NR₅OR₅, —X₁C(O)R₅, —X₁OX₂OR₅, —OX₁R₅, —X₁R₅, —X₁C(O)OR₅, —X₁OR₅ and —X₁OX₁OR₅; wherein each X₁ is independently selected from a bond and C₁₋₄alkylene; X₂ is C₁₋₄alkylene; and each R₅ is independently selected from hydrogen, C₁₋₆alkyl, C₂₋₄alkenyl, C₃₋₁₂cycloalkyl, C₆₋₁₀aryl-C₀₋₄alkyl, heteroaryl-C₀₋₄alkyl and heterocyclyl;
wherein said aryl, cycloalkyl, heteroaryl or heterocyclyl substituents of R₄ can optionally be further substituted with 1 to 3 radicals independently selected from halo, hydroxy, cyano, C₁₋₆alkyl, C₁₋₆alkoxy, halo-substituted-C₁₋₆alkyl, halo-substituted-C₁₋₆alkoxy, -L-OR₆, -L-C(O)OR₆, -L-C(O)NR₆R₆ and -L-R₆; wherein L is selected from a bond and C₁₋₄alkylene; and R₆ is selected from hydrogen, C₁₋₆alkyl and heterocyclyl;
R₇ is hydrogen;
R₈ is selected from hydrogen, halo, methoxy, amino, difluoromethoxy, trifluoromethyl, pyrrolidinyl, morpholino, 2-methyl-morpholino, 2,6-dimethyl-morpholino, cyano, —NR₅ₐR_{5b} and methyl; or R₇ and R₈ together with the carbon atoms to, which R₇ and R₈ are attached form phenyl; wherein R₅ₐ and R_{5b} are independently selected from hydrogen, C₁₋₆alkyl, C₁₋₆alkoxy, halo-substituted-C₁₋₄alkyl and halo-substituted-C₁₋₆alkoxy;
R₉ is selected from hydrogen, morpholino, halo, C₁₋₆alkyl, C₁₋₆alkoxy, halo-substituted-C₁₋₆alkyl, halo-substituted-C₁₋₆alkoxy, —NR₅ₐR_{5b}, OX₁NR₅ₐR_{5b} and heterocyclyl; wherein X₁ is independently selected from a bond and C₁₋₄alkylene; and R₅ₐ and R_{5b} are independently selected from hydrogen, C₁₋₆alkyl, C₁₋₆alkoxy, halo-substituted-C₁₋₆alkyl and halo-substituted-C₁₋₆alkoxy.

3. The compound of claim 2 in which: R₃ is methyl; and R₄ is pyrazolyl, pyridinyl, indolyl, indolin-2-yl, thienyl, thiazolyl, 3-oxo-3,4-dihydro-2H-benzo[b][1,4]oxazin-6-yl, furanyl, benzo[b]furanyl, 1,3,4-thiadiazolyl, benzo[b]thiophenyl, pyrrolyl, 1H-indazolyl, imidazo[1,2-a]pyridin-3-yl, oxazolyl, benzo[d]thiazol-6-yl, 1H-benzo[d][1,2,3]triazol-5-yl, quinolinyl, 1H-indolyl, 3,4-dihydro-2H-pyrano[2,3-b]pyridinyl, 3-oxo-3,4-dihydro-2H-benzo[b][1,4]oxazin-7-yl and 2,3-dihydrofuro[2,3-b]pyridinyl;
wherein said heteroaryls of R₄ are substituted with 1 to 3 radicals independently selected from halo, hydroxy, cyano, methyl, amino, phenyl, hydroxy-ethyl(methyl)amino, piperidinyl, trifluoromethyl, 2-methyl allyloxy, cyclopropyl-methyl (propyl)amino-methyl, trifluoromethoxy, 3,4-dihydroisoquinolin-2(1H)-yl, amino-carbonyl-methyl(ethyl)amino-methyl, pyridinyl-methyl(ethyl)-amino-methyl, isopropyl(ethyl)-amino-methyl, propyl(ethyl)-amino-methyl, morpholino, butyl(methyl)amino-methyl, isobutyl(methyl)amino-methyl, benzyl(ethyl)amino-methyl, pyridinyl, pyrrolidinyl, azepanyl, hydroxy-propyloxy, ethyl, methoxy, methyl-carbonyl, ethoxy, propyloxy, t-butyl, benzyl, propyl, isopropyloxy, isopropyl, diethylamino-sulfonyl, methyl-sulfonyl, isopropyl-sulfonyl, diethyl-amino-methyl, trifluoroethoxy, piperidinyl, isoquinolinyl, (hydroxy-ethyl)(methyl)amino, difluoro-ethoxy, cyclopropyl, cyclopropyl-methoxy and tetrahydrofuranyl-oxy;
wherein said aryl, cycloalkyl, heteroaryl or heterocyclyl substituents of R₄ can optionally be further substituted with 1 to 3 radicals independently selected from halo, methyl, pyrrolidinyl-methyl, trifluoromethyl, hydroxy-methyl, hydroxy and cyano.

4. The compound of claim 3 in which R₉ is selected from hydrogen and dimethyl-amino-propyloxy.

5. The compound of claim 4 selected from: N-(3-(4-(pyridin-3-yl)pyrimidin-2-ylamino)-4-methylphenyl)-5-chloro-1H-indole-2-carboxamide; N-(3-(4-(pyridin-3-yl)pyrimidin-2-ylamino)-4-methylphenyl)-1-ethyl-3-methyl-1H-pyrazole-5-carboxamide; N-(3-(4-(pyridin-3-yl)pyrimidin-2-ylamino)-4-methylphenyl)-1,3-dimethyl-1H-pyrazole-5-carboxamide; N-(3-(4-(pyridin-3-yl)pyrimidin-2-ylamino)-4-methylphenyl)-5-(trifluoromethyl)-2-methyloxazole-4-carboxamide; N-(3-(4-(pyridin-3-yl)pyrimidin-2-ylamino)-4-methylphenyl)-2-morpholinopyridine-4-carboxamide; N-(3-(4-(pyridin-3-yl)pyrimidin-2-ylamino)-4-methylphenyl)-6-methoxypyridine-3-carboxamide; N-(3-(4-(pyridin-3-yl)pyrimidin-2-ylamino)-4-methylphenyl)-6-methoxypyridine-3-carboxamide; N-(3-(4-(pyridin-3-yl)pyrimidin-2-ylamino)-4-methylphenyl)-1,5-dimethyl-1H-pyrazole-3-carboxamide; N-(3-(4-(5-methylpyridin-3-yl)pyrimidin-2-ylamino)-4-methylphenyl)-1,5-dimethyl-1H-pyrazole-3-carboxamide; N-(3-(4-(5-methoxypyridin-3-yl)pyrimidin-2-ylamino)-4-methylphenyl)-1,5-dimethyl-1H-pyrazole-3-carboxamide; 2-(2,2-difluoroethoxy)-N-(3-(4-(pyridin-3-yl)pyrimidin-2-ylamino)-4-methylphenyl)pyridine-4-carboxamide; 6-(2,2,2-trifluoroethoxy)-N-(3-(4-(pyridin-3-yl)pyrimidin-2-ylamino)-4-methylphenyl)pyridine-3-carboxamide; 3-(4-(pyridin-3-yl)pyrimidin-2-ylamino)-N-(3,4-dihydro-3-oxo-2H-benzo[b][1,4]oxazin-6-yl)-4-methylbenzamide; and N-(3-(4-(5-methoxypyridin-3-yl)pyrimidin-2-ylamino)-4-methylphenyl)-1,5-dimethyl-1H-pyrazole-3-carboxamide.

6. A pharmaceutical composition comprising a therapeutically effective amount of a compound of claim 1 in combination with a pharmaceutically acceptable excipient.

7. The pharmaceutical composition of claim 6, wherein the pharmaceutically acceptable excipient is suitable for parenteral administration.

8. The pharmaceutical composition of claim 6, wherein the pharmaceutically acceptable excipient is suitable for oral administration.

9. A method for modulating kinase activity, comprising administering to a system or a subject in need thereof, a therapeutically effective amount of the compound of claim 1 or pharmaceutically acceptable salts or pharmaceutical compositions thereof, thereby modulating said kinase activity.

10. The method of claim 9, wherein said kinase is selected from c-kit, Abl, Lyn, MAPK14 (p38delta), PDGFRα, PDGFRβ, ARG, BCR-Abl, BRK, EphB, Fms, Fyn, KDR, LCK, PDGF-R, b-Raf, c-Raf, SAPK2, Src, Tie2 and TrkB, or a combination thereof.

11. The method of claim 9, wherein said kinase is c-kit kinase receptor.

12. The method of claim 11, wherein the compound of claim 1 directly contacts the c-kit, PDGFRα and/or PADGRβ kinase receptors.

13. The method of claim 12, wherein the contacting occurs in vitro or in vivo.

14. A method for treating a disease or condition wherein modulation of kinase activity can prevent, inhibit or ameliorate the pathology and/or symptomology of the disease or condition, comprising administering to a subject a therapeutically effective amount of the compound of claim 1 or pharmaceutically acceptable salts or pharmaceutical compositions thereof, and optionally a therapeutically effective amount of a second agent.

15. The method of claim 14, wherein said kinase is selected from c-kit, PDGFRα and PADGRβ kinase receptors.

16. The method of claim 14, wherein the second agent is a bronchodilator, an anti-inflammatory agent, a leukotriene antagonist, or an IgE blocker.

17. The method of claim 14, wherein the compound of claim 1 is administered prior to, simultaneously with, or after the second agent.

18. The method of claim 14, wherein said disease or condition is a neoplastic disorder, an allergy disorder, an inflammatory disorder, an autoimmune disorder, a Plasmodium related disease, a mast cell associated disease, a graft-versus-host disease, a metabolic syndrome, a CNS related disorder, a neurodegenerative disorder, a pain condition, a substance abuse disorder, a prion disease, a cancer, a heart disease, a fibrotic disease, idiopathic arterial hypertension (IPAH), or primary pulmonary hypertension (PPH).

19. The method of claim 18, wherein the neoplastic disorder is mastocytosis, gastrointestinal stromal tumor, small cell lung cancer, non-small cell lung cancer, acute myelocytic leukemia, acute lymphocytic leukemia, myelodyplastic syndrome, chronic myelogenous leukemia, colorectal carcinoma, gastric carcinoma, testicular cancer, glioblastoma or astrocytoma.

20. The method of claim 18, wherein the allergy disorder is asthma, allergic rhinitis, allergic sinusitis, anaphylactic syndrome, urticaria, angioedema, atopic dermatitis, allergic contact dermatitis, erythema nodosum, erythema multiforme, cutaneous necrotizing venulitis, insect bite skin inflammation, or blood sucking parasite infestation.

21. The method of claim 18, wherein the inflammatory disorder is rheumatoid arthritis, conjunctivitis, rheumatoid spondylitis, osteoarthritis or gouty arthritis.

22. The method of claim 18, wherein the autoimmune disorder is multiple sclerosis, psoriasis, intestine inflammatory disease, ulcerative colitis, Crohn's disease, rheumatoid arthritis, polyarthritis, local or systemic scleroderma, systemic lupus erythematosus, discoid lupus erythematosis, cutaneous lupus, dermatomyositis, polymyositis, Sjogren's syndrome, nodular panarteritis, autoimmune enteropathy or proliferative glomerulonephritis.

23. The method of claim 18, wherein the graft-versus-host disease is organ transplantation graft rejection.

24. The method of claim 18, wherein the organ transplantation is kidney transplantation, pancreas transplantation, liver transplantation, heart transplantation, lung transplantation, or bone marrow transplantation.

25. The method of claim 18, wherein the metabolic syndrome is type I diabetes, type II diabetes, or obesity.

26. The method of claim 18, wherein the CNS related disorder is depression, dysthymic disorder, cyclothyinic disorder, anorexia, bulimia, premenstrual syndrome, post-menopause syndrome, mental slowing, loss of concentration, pessimistic worry, agitation, self-deprecation and decreased libido, an anxiety disorder, a psychiatric disorder or schizophrenia.

27. The method of claim 18, wherein the neurodegenerative disorder is Alzheimer's disease, Parkinson's disease, Huntington's disease, the prion diseases, Motor Neuron Disease (MND), or Amyotrophic Lateral Sclerosis (ALS).

28. The method of claim 18, wherein the pain condition is acute pain, postoperative pain, chronic pain, nociceptive pain, cancer pain, neuropathic pain or psychogenic pain syndrome.

29. The method of claim 18, wherein the substance use disorder is drug addiction, drug abuse, drug habituation, drug dependence, withdrawal syndrome or overdose.

30. The method of claim 18, wherein the cancer is melanoma, gastrointestinal stromal tumor (GIST), small cell lung cancer, or other solid tumors.

31. The method of claim 18, wherein the fibrotic disease is hepatitis C (HCV), liver fibrosis, nonalcoholic steatohepatitis (NASH), cirrhosis in liver, pulmonary fibrosis, or bone marrow fibrosis.

32. The method of claim 18, wherein the Plasmodium related disease is malaria.
